SEQUENCE LISTING (5 sequences)

SEQ ID NO: 1 - DNA, 16 bp, Synthetic construct
  source 1..16: mol_type = other DNA
  modified_base 1: mod_base = OTHER; note = 2'-O,4'-C-methylene guanosine 3'-phosphorothioate
  modified_base 2: mod_base = OTHER; note = 2'-O,4'-C-methylene 5-methylcytidine 3'-phosphorothioate
  modified_base 3: mod_base = OTHER; note = 2'-O,4'-C-methylene adenosine 3'-phosphorothioate
  modified_base 4: mod_base = OTHER; note = 2'-deoxythymidine 3'-phosphorothioate
  modified_base 5: mod_base = OTHER; note = 2'-deoxythymidine 3'-phosphorothioate
  modified_base 6: mod_base = OTHER; note = 2'-deoxy 5-methylcytidine 3'-phosphorothioate
  modified_base 7: mod_base = OTHER; note = 2'-deoxythymidine 3'-phosphorothioate
  modified_base 10: mod_base = OTHER; note = 2'-deoxythymidine 3'-phosphorothioate
  modified_base 8: mod_base = OTHER; note = 2'-deoxyadenosine 3'-phosphorothioate
  modified_base 9: mod_base = OTHER; note = 2'-deoxyadenosine 3'-phosphorothioate
  modified_base 11: mod_base = OTHER; note = 2'-deoxyadenosine 3'-phosphorothioate
  modified_base 12: mod_base = OTHER; note = 2'-deoxyguanosine 3'-phosphorothioate
  modified_base 13: mod_base = OTHER; note = 2'-deoxy 5-methylcytidine 3'-phosphorothioate
  modified_base 14: mod_base = OTHER; note = 2'-O,4'-C-methylene adenosine 3'-phosphorothioate
  modified_base 15: mod_base = OTHER; note = 2'-O,4'-C-methylene guanosine 3'-phosphorothioate
  modified_base 16: mod_base = OTHER; note = 2'-O,4'-C-methylene 5-methylcytidine
gcattctaatagcagc

SEQ ID NO: 2 - RNA, 23 nt, Synthetic construct
  source 1..23: mol_type = other RNA
  modified_base 2: mod_base = OTHER; note = 2'-O-methylguanosine 5'-phosphorothioate
  modified_base 3: mod_base = cm
  modified_base 5: mod_base = cm
  modified_base 8: mod_base = cm
  modified_base 18: mod_base = cm
  modified_base 4: mod_base = um
  modified_base 13: mod_base = um
  modified_base 14: mod_base = um
  modified_base 17: mod_base = um
  modified_base 21: mod_base = um
  modified_base 6: mod_base = OTHER; note = 2'-O-methyladenosine
  modified_base 7: mod_base = OTHER; note = 2'-O-methyladenosine
  modified_base 9: mod_base = OTHER; note = 2'-O-methyladenosine
  modified_base 16: mod_base = OTHER; note = 2'-O-methyladenosine
  modified_base 19: mod_base = OTHER; note = 2'-O-methyladenosine
  modified_base 22: mod_base = OTHER; note = 2'-O-methyladenosine 3'-phosphorothioate
  modified_base 15: mod_base = gm
  modified_base 20: mod_base = gm
  modified_base 11: mod_base = OTHER; note = 2'-fluoroadenosine
  modified_base 10: mod_base = OTHER; note = 2'-fluorouridine
  modified_base 12: mod_base = OTHER; note = 2'-fluorouridine
  modified_base 1: mod_base = OTHER; note = Inverted abasic site
  modified_base 23: mod_base = OTHER; note = Inverted abasic site
ngctcaacatatttgatcagtan

SEQ ID NO: 3 - RNA, 21 nt, Synthetic construct
  source 1..21: mol_type = other RNA
  modified_base 1: mod_base = OTHER; note = 2'-O-methyluridine 3'-phosphorothioate
  modified_base 4: mod_base = OTHER; note = 2'-fluorouridine
  modified_base 12: mod_base = OTHER; note = 2'-fluorouridine
  modified_base 14: mod_base = OTHER; note = 2'-fluorouridine
  modified_base 16: mod_base = OTHER; note = 2'-fluorouridine
  modified_base 17: mod_base = um
  modified_base 2: mod_base = OTHER; note = 2'-fluoroadenosine 3'-phosphorothioate
  modified_base 6: mod_base = OTHER; note = 2'-fluoroadenosine
  modified_base 10: mod_base = OTHER; note = 2'-fluoroadenosine
  modified_base 9: mod_base = OTHER; note = 2'-O-methyladenosine
  modified_base 11: mod_base = OTHER; note = 2'-O-methyladenosine
  modified_base 13: mod_base = OTHER; note = 2'-O-methyladenosine
  modified_base 19: mod_base = OTHER; note = 2'-O-methyladenosine
  modified_base 18: mod_base = OTHER; note = 2'-fluoroguanosine
  modified_base 20: mod_base = OTHER; note = 2'-fluoroguanosine 3'-phosphorothioate
  modified_base 8: mod_base = OTHER; note = 2'-fluorocytidine
  modified_base 3: mod_base = OTHER; note = 2'-O-methylcytidine 3'-phosphorothioate
  modified_base 5: mod_base = gm
  modified_base 15: mod_base = gm
  modified_base 21: mod_base = cm
  modified_base 7: mod_base = um
tactgatcaaatatgttgagc

SEQ ID NO: 4 - RNA, 18 nt, Synthetic construct
  source 1..18: mol_type = other RNA
  modified_base 1: mod_base = OTHER; note = 2'-O-methylcytidine 3'-phosphorothioate
  modified_base 11: mod_base = OTHER; note = 2'-O-methylcytidine
  modified_base 2: mod_base = OTHER; note = 2'-fluoroadenosine 3'-phosphorothioate
  modified_base 6: mod_base = OTHER; note = 2'-fluoroadenosine
  modified_base 12: mod_base = OTHER; note = 2'-fluoroadenosine
  modified_base 18: mod_base = OTHER; note = 2'-fluoroadenosine
  modified_base 3: mod_base = gm
  modified_base 15: mod_base = gm
  modified_base 17: mod_base = OTHER; note = 2'-O-methylguanosine 3'-phosphorothioate
  modified_base 4: mod_base = OTHER; note = 2'-fluorocytidine
  modified_base 10: mod_base = OTHER; note = 2'-fluorocytidine
  modified_base 14: mod_base = OTHER; note = 2'-fluorocytidine
  modified_base 5: mod_base = OTHER; note = 2'-O-methyladenosine
  modified_base 7: mod_base = OTHER; note = 2'-O-methyladenosine
  modified_base 8: mod_base = OTHER; note = 2'-fluorouridine
  modified_base 16: mod_base = OTHER; note = 2'-fluorouridine 3'-phosphorothioate
  modified_base 9: mod_base = um
  modified_base 13: mod_base = um
cagcaaattccatcgtga

SEQ ID NO: 5 - RNA, 20 nt, Synthetic construct
  source 1..20: mol_type = other RNA
  modified_base 1: mod_base = OTHER; note = 5'-phosphate 2'-O-methyluridine 3'-phosphorothioate
  modified_base 19: mod_base = OTHER; note = 2'-O-methyluridine 3'-phosphorothioate
  modified_base 17: mod_base = um
  modified_base 13: mod_base = um
  modified_base 7: mod_base = um
  modified_base 2: mod_base = OTHER; note = 2'-fluorocytidine 3'-phosphorothioate
  modified_base 4: mod_base = OTHER; note = 2'-fluorocytidine
  modified_base 16: mod_base = OTHER; note = 2'-fluorocytidine
  modified_base 6: mod_base = OTHER; note = 2'-fluoroadenosine
  modified_base 10: mod_base = OTHER; note = 2'-fluoroadenosine
  modified_base 3: mod_base = OTHER; note = 2'-O-methyladenosine
  modified_base 11: mod_base = OTHER; note = 2'-O-methyladenosine
  modified_base 5: mod_base = gm
  modified_base 9: mod_base = gm
  modified_base 15: mod_base = gm
  modified_base 8: mod_base = OTHER; note = 2'-fluoroguanosine
  modified_base 18: mod_base = OTHER; note = 2'-fluoroguanosine 3'-phosphorothioate
  modified_base 12: mod_base = OTHER; note = 2'-fluorouridine
  modified_base 14: mod_base = OTHER; note = 2'-fluorouridine
  modified_base 20: mod_base = OTHER; note = 2'-fluorouridine
tcacgatggaatttgctgtt

(19) United States
(12) Patent Application Publication
(10) Pub. No.: US 2025/0051771 A1
(43) Pub. Date: Feb. 13, 2025

(54) MULTIVALENT CARGO-CARRYING COMPLEXES AND USES THEREOF

(71) Applicant: ASTRAZENECA AB, Södertälje (SE)

(72) Inventors: Lars Anders DAHLÉN, Gothenburg (SE); Tom BALADI, Gothenburg (SE)

(21) Appl. No.: 18/780,800

(22) Filed: Jul. 23, 2024

(30) Foreign Application Priority Data
Jul. 24, 2023 (GB) 2311330.1
May 23, 2024 (GB) 2407370.2

(51) Int. Cl. C12N 15/113

(52) U.S. Cl. CPC C12N 15/113; C12N 2310/14; C12N 2310/351; C12N 2310/51

(57) ABSTRACT

Provided are multivalent saccharide-containing compounds which are bonded to nucleic acids. These compounds comprise one or more nitrogen-modified phosphate groups which link the cargo moieties (e.g., therapeutic nucleic acids) and/or the ligands (e.g., GalNAc) to the core of the complex. The compounds are useful for delivering nucleic acids to cells or tissues, e.g. for use in therapeutic treatments. Also provided are pharmaceutical compositions comprising the aforementioned compounds and medical uses of the same, including their use in treating or preventing conditions such as liver diseases.

Multivalent saccharide-containing compounds are provided which are bonded to nucleic acids. These compounds are useful for delivering nucleic acids to cells or tissues, e.g. for use in therapeutic treatments. In particular, the compounds comprise specific monosaccharides which are capable of binding to asialoglycoprotein receptors (ASGPRs). Such compounds may be used, for example, in the targeted delivery of therapeutic oligonucleotides to cells such as liver cells. Also provided are pharmaceutical compositions comprising the aforementioned compounds and medical uses of the same, including their use in treating or preventing conditions such as liver diseases.

### CROSS REFERENCE TO RELATED APPLICATIONS

This specification claims the benefit of priority to GB Patent Application Nos. 2311330.1 (filed 24 Jul. 2023) and 2407370.2 (filed 23 May 2024). The entire texts of the above-referenced patent applications are incorporated by reference into this specification.

### SUMMARY

There are numerous ways to target the delivery of cargo moieties, including therapeutic nucleic acids, to cells. One approach is to tether the cargo to one or more saccharide ligands to take advantage of saccharide-binding proteins associated with the target cells. Exemplary saccharide-binding proteins include cell surface receptors such as glycoprotein receptors, including asialoglycoprotein receptors (ASGPRs). ASGPRs have been shown to be highly expressed on the surface of mammalian hepatocytes, as well as carcinoma cell lines; they are also expressed on other cell types, albeit generally at a lower level. As such, ASGPRs represent a promising target for hepatic delivery of cargo, including therapeutic nucleic acid agents (see, e.g., D'Souza et al., J. Control Release, (2015) 203:126-139). Ligands which can be used for targeting ASGPRs include monosaccharides such as N-acetyl galactosamine (GalNAc).

Target-binding complexes often contain more than one ligand and/or more than one cargo moiety. This functionality can be achieved by the use of branched core structures with linkers being used to couple the ligands and/or the cargo moieties to the core. In this way, a complex can be prepared which has the appropriate number and arrangement of functional parts for the desired use. Examples of multivalent, e.g. branched, complexes are described in International Patent Publications: WO 2014/179620 (Isis Pharmaceuticals, Inc.); WO 2015/177668 A1 (Pfizer Inc.); WO 2009/073809 A2 (Alnylam Pharmaceuticals, Inc.); WO 2012/083046 A2 (Arrowhead Research Corporation); WO 2017/156012 A1 (Arrowhead Pharmaceuticals, Inc.); WO 2016/100401 A1 (Dicerna Pharmaceuticals, Inc.); WO 2017/174657 A1 (Silence Therapeutics GmbH); and WO 2019/092280 A1 (Silence Therapeutics GmbH). Complexes of the aforementioned types make use of different coupling chemistries to link the ligands and cargo moieties to the branched core structure, although phosphate groups are conventionally used for the coupling of cargo moieties (e.g., nucleic acids) within such complexes. Phosphate groups are non-toxic and are generally amenable to chemical modification, but may not present optimum characteristics when used in complexes of the type described herein. There is, therefore, a need for alternative chemical functionalities to couple cargo moieties and/or ligands to core structures in order to prepare complexes useful in delivering cargo moieties to cells. The present disclosure seeks to address this need by providing novel compounds and complexes, e.g. for use in targeting therapeutic nucleic acids to cells such as liver cells.

In brief, the compounds and complexes of the present disclosure contain one or more nitrogen-modified phosphate groups which link the cargo moieties (e.g., therapeutic nucleic acids) and/or the ligands (e.g., GalNAc) to the core of the complex. The nitrogen-modified phosphate groups include phosphoryl guanidines and N-sulfonylphosphoramidates. Without wishing to be bound by theory, it is postulated that the use of these modified phosphate groups as linkers facilitates the synthesis of complexes having good stability, low aggregation, low toxicity, favourable pharmacokinetics, and/or good potency. In particular, the compounds and complexes of the present disclosure may demonstrate enhanced stability, especially chiral stability, as compared to conventional phosphate-containing compounds and complexes. The compounds and complexes of the present disclosure may also exhibit a preferential targeting to specific tissue types; for example, the GalNAc-containing compounds and complexes described herein which contain nitrogen-modified phosphate groups may be targeted preferentially to liver tissue over kidney tissue. The compounds and complexes of the present disclosure may therefore accumulate more in certain tissue types (e.g., in liver) which can in turn reduce unwanted effects of the cargo (e.g., pharmacological effects) in non-target tissue. Side effects of the therapeutic compounds and complexes of the present disclosure may therefore be reduced as compared to, e.g., conventional phosphate- or thiophosphate-containing compounds.

### SUMMARY OF THE DISCLOSURE

The present disclosure includes the following aspects and embodiments which are presented as numbered clauses 1 to 57:

1. A nucleic acid delivery agent, which is a compound, or a pharmaceutically acceptable salt thereof, wherein the compound comprises a nucleic acid and at least one moiety having the structure of Formula (I):

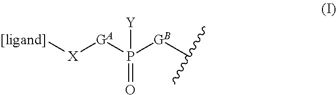

wherein:
X is selected from optionally substituted —(C₁-C₁₀)alkylene-, —(C₁-C₁₀)alkenylene- and *—[(CH₂)₂O]ₒ(CH₂)₂— (wherein * denotes the point of attachment of the group to G^{A}, and wherein o is an integer selected from 1, 2, and 3);
G^{A} and G^{B} are each independently selected from O and NH, provided that at least one of G^{A} and G^{B} is O;
Y is an —NH-sulfonyl group or a guanidine-containing moiety; and
[ligand] denotes a monosaccharide.

2. The nucleic acid delivery agent of clause 1, wherein the ligand is N-acetyl galactosamine (GalNAc).

3. The nucleic acid delivery agent of clause 1, wherein the moiety having the structure of Formula (I) is a moiety having the structure of Formula (II):

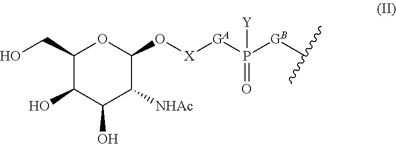

wherein X, G^{A} G^{B}, and Y are as defined in clause 1.

4. The nucleic acid delivery agent of any one of the preceding clauses, wherein X is selected from optionally substituted —(C₃-C₆)alkylene- and *—[(CH₂)₂O]ₒ(CH₂)₂— (wherein * denotes the point of attachment of the group to G^{A}, and wherein o is an integer selected from 1, 2, and 3).

5. The nucleic acid delivery agent of any one of the preceding clauses, wherein X is the alkylene group —(CH₂)ₘ— wherein m is an integer selected from 3, 4, 5 and 6 (e.g., 4), or wherein X is —CH₂CH₂OCH₂CH₂—.

6. The nucleic acid delivery agent of any one of the preceding clauses, wherein G^{A} and G^{B} are each independently O.

7. The nucleic acid delivery agent of any one of the preceding clauses, wherein Y is an N-sulfonyl group, e.g. an —NH-alkylsulfonyl group or an —NH-arylsulfonyl group, wherein alkyl denotes optionally substituted —(C₁-C₆)alkyl and aryl denotes optionally substituted phenyl.

8. The nucleic acid delivery agent of clause 7, wherein Y is selected from —NH-mesyl, —NH-phenylsulfonyl and —NH-tosyl.

9. The nucleic acid delivery agent of any one of clauses 1-6, wherein Y is a guanidine-containing moiety, e.g. wherein Y is a 1,3-dimethyl-2-(imino)imidazolidine group which is bonded to phosphorus via the imino nitrogen atom.

10. A compound, or a pharmaceutically acceptable salt thereof, wherein the compound comprises: one or more moieties having a structure independently selected from Formula (I) and Formula (II) as defined in any one of the preceding clauses; and one or more cargo moiety, wherein each cargo moiety comprises (e.g., is) a nucleic acid.

11. The compound or pharmaceutically acceptable salt thereof of clause 10 wherein the compound has the structure of Formula (III):

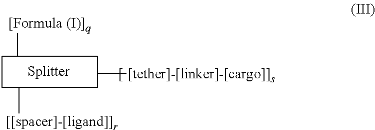

wherein each Formula (I) group has a structure independently selected from Formula (I) as defined in any one of clauses 1-9, and wherein:
q is an integer selected from 1, 2, 3 and 4;
r is an integer selected from 0, 1, 2 and 3;
s is an integer selected from 1 and 2;
[ligand] in each case independently denotes a monosaccharide;
[spacer] in each case independently denotes a moiety comprising a chain of atoms (e.g., a chain of carbon atoms with one or more heteroatoms selected independently from N, O, S, and P, optionally intervening) that serves to link the ligand to the splitter;
Splitter denotes a moiety having at least 2 (e.g., 2, 3, 4 or 5) points of attachment for the Formula (I), [spacer]-[ligand] and [tether]-[linker]-[cargo] moieties (where present);
[tether] and [linker] taken together in each case independently denote a moiety comprising a chain of atoms (e.g., a chain of carbon atoms with one or more heteroatoms selected independently from N, O, S, and P optionally intervening) that serves to link the cargo to the splitter; and
[cargo] denotes the cargo moiety.

12. The compound or pharmaceutically acceptable salt thereof of clause 11, wherein the ligand in each case is GalNAc.

13. The compound or pharmaceutically acceptable salt thereof of clause 11 or clause 12, wherein the sum of q, r and s is 3, 4 or 5.

14. The compound or pharmaceutically acceptable salt thereof of any one of clauses 11-13, wherein each spacer (where present) independently has a structure which comprises (e.g., which is):

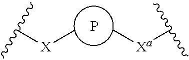

wherein:
X is as defined in any one of clauses 1-5;
X^{a} is optionally substituted —(CH₂)ₜ— or —C(O)(CH₂)ᵤ—* (wherein * denotes the point of attachment to the group “P”), wherein t and u are integers independently selected from 1, 2, 3, 4, 5 and 6 (e.g., 2, 3, 4 or 5); and
group “P” is selected from phosphate, thiophosphate, and phosphoramidate.

15. The compound or pharmaceutically acceptable salt thereof of clause 14, wherein X is —(CH₂)ₘ—, or *—[(CH₂)₂O]ₒ(CH₂)₂— (wherein * denotes the point of attachment of the group to “P”), wherein m is an integer selected from 3, 4 and 5, and o is an integer selected from 1, 2 and 3.

16. The compound or pharmaceutically acceptable salt thereof of any one of clauses 11-15, wherein the Splitter is (or comprises) the group:

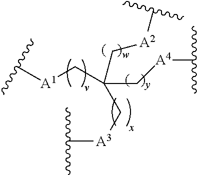

wherein:
A¹, A², A³ and A⁴ are each independently selected from —O—, —S—, —NH—, —CH₂—, and —C(O)—; and

v, w, x and y are each an integer independently selected from 0, 1, 2 and 3.

17. The compound or pharmaceutically acceptable salt thereof of clause 16, wherein each of A¹, A², A³ and A⁴ is —O—.

18. The compound or pharmaceutically acceptable salt thereof of clause 16 or clause 17, wherein each of v, w, x and y is 1.

19. The compound or pharmaceutically acceptable salt thereof of any one of clauses 16-18, wherein the Formula (I) and [spacer]-[ligand] moieties (when present) are connected to the splitter via A¹, A² and A³, and a [tether]-[linker]-[cargo] moiety is connected to the splitter via A⁴.

20. The compound or pharmaceutically acceptable salt thereof of any one of clauses 10-19, wherein each cargo moiety is independently selected from an antisense oligonucleotide (ASO), an immunostimulatory oligonucleotide, a decoy oligonucleotide, a splice altering oligonucleotide, a splice-switching oligonucleotide, a triplex forming oligonucleotide, a siRNA, a saRNA, a microRNA, a microRNA mimic, an anti-miR, a double stranded RNA, a single stranded RNA, a ribozyme, an aptamer, a spiegelmer, a CRISPR oligonucleotide and a G-quadruplex.

21. The compound or pharmaceutically acceptable salt thereof of clause 20, wherein each cargo moiety is an antisense oligonucleotide, or wherein each cargo moiety is a siRNA.

22. A compound, or a pharmaceutically acceptable salt thereof, wherein the compound has the structure of Formula (IV):

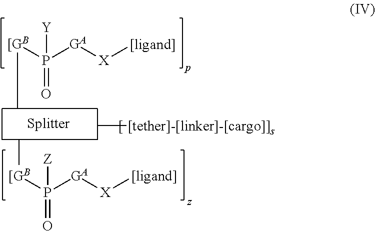

wherein X, Y, G^{A} G^{B}, s, Splitter, [ligand], [tether], [linker] and [cargo] are as defined in any of the preceding clauses, and wherein:
p and z are each independently an integer selected from 0, 1, 2, 3 and 4, wherein the sum of p and z is 2, 3 or 4; and
Z in each case is independently selected from —SH and —OH,

with the proviso that when p is 0, at least one [tether]-[linker] moiety comprises a phosphoryl guanidine or N-sulfonylphosphoramidate group.

23. The compound or pharmaceutically acceptable salt thereof of clause 22, wherein X in each case is independently optionally substituted —(CH₂)ₘ— or *—[(CH₂)₂O]ₒ(CH₂)₂— (wherein * denotes the point of attachment of the group to G^{A}), wherein each m is independently an integer selected from 3, 4, 5 and 6, and each o is independently an integer selected from 1, 2 and 3.

24. The compound or pharmaceutically acceptable salt thereof of clause 22 or clause 23, wherein p is an integer selected from 1, 2, 3 and 4, and z is an integer selected from 0, 1, 2 and 3, optionally wherein the sum of p and z is 2 or 3, and s is 1.

25. The compound or pharmaceutically acceptable salt thereof of clause 22 or clause 23, wherein p is 0, and z is an integer selected from 2, 3 and 4, optionally wherein z is 2 or 3, and s is 1.

26. A compound, or a pharmaceutically acceptable salt thereof, wherein the compound has the structure of Formula (V):

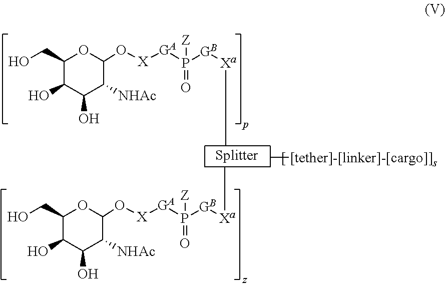

wherein X, Y, Z, X^{a}, G^{A}, G^{B}, p, s, z, Splitter, [tether], [linker] and [cargo] are as defined in any of the preceding clauses, and wherein the sum of p and z is 2, 3 or 4, with the proviso that when p is 0, at least one [tether]-[linker] moiety comprises a phosphoryl guanidine or N-sulfonylphosphoramidate group.

27. The compound or pharmaceutically acceptable salt thereof of clause 26, wherein X in each case is independently optionally substituted —(CH₂)ₘ— or *—[(CH₂)₂O]ₒ(CH₂)₂— (wherein * denotes the point of attachment of the group to G^{A}), wherein each m is independently an integer selected from 3, 4, 5 and 6, and each o is independently an integer selected from 1, 2 and 3.

28. The compound or pharmaceutically acceptable salt thereof of clause 26 or clause 27, wherein X^{a} in each case is independently optionally substituted —(CH₂)ₜ— or —C(O)(CH₂)ᵤ—* (wherein * denotes the point of attachment to G^{B}), and wherein t and u in each case are integers independently selected from 1, 2, 3, 4, 5 and 6.

29. The compound or pharmaceutically acceptable salt thereof of any one of clauses 22-28, wherein:
a) Y in each case is independently selected from

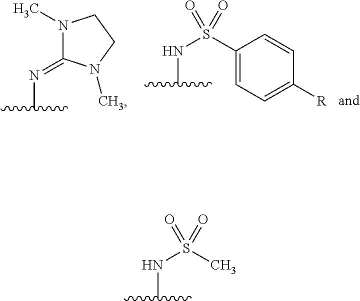

wherein R is independently —CH₃ or —H; and/or
b) said N-sulfonylphosphoramidate group is independently selected from

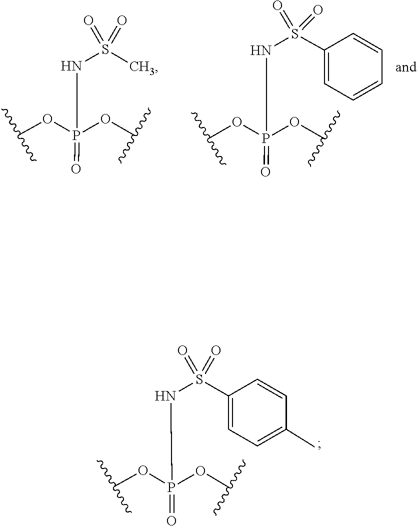 [SMILES: *OP(=O)(NS(C)(=O)=O)O* ; *OP(=O)(NS(=O)(=O)c1ccccc1)O* ; *OP(=O)(NS(=O)(=O)c1ccc(C)cc1)O*]

and/or
c) said phosphoryl guanidine group is independently

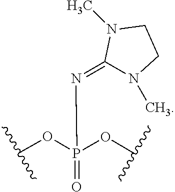 [SMILES: *OP(=O)(N=C1N(C)CCN1C)O*]

30. The compound or pharmaceutically acceptable salt thereof of any one of clauses 11-29, wherein each [tether]-[linker]-[cargo] moiety is independently selected from:

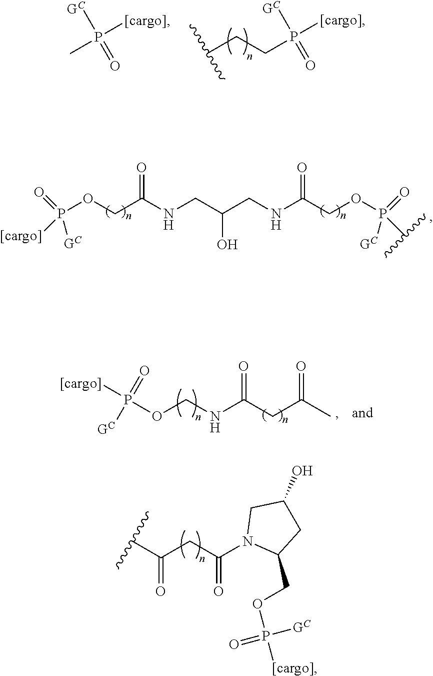

wherein:
n is independently an integer between 1 and 12 inclusive;
G^{C} is selected from Y and Z, wherein
   Y is an N-sulfonyl group or a guanidine-containing moiety, and
   Z is selected from —SH and —OH; and
[cargo] is a nucleic acid.

31. A compound, or a pharmaceutically acceptable salt thereof, wherein the compound is of Formula (VI):

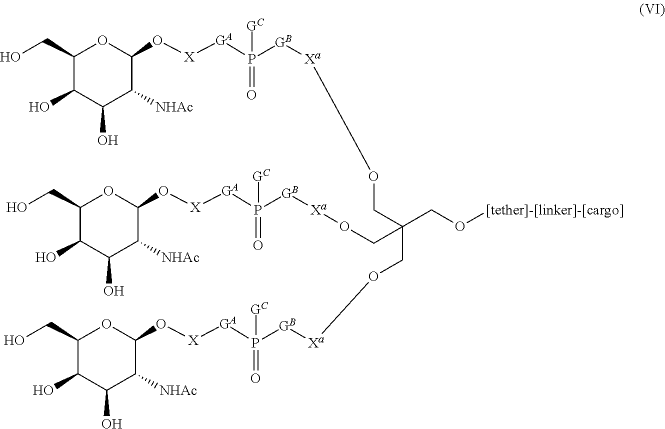

wherein X, G^{A} G^{B}, X^{a}, [tether], [linker] and [cargo] are as defined in any of the preceding clauses, and G^{C} in each case is independently selected from Y and Z as defined in any of the preceding clauses.

32. The compound or pharmaceutically acceptable salt thereof of clause 31, wherein G^{A} and G^{B} in each case are O, and wherein all three of the G^{C} groups are independently selected from Y.

33. The compound or pharmaceutically acceptable salt thereof of clause 31 or clause 32, wherein X in each case is independently —(CH₂)ₘ— wherein each m is independently an integer selected from 3, 4, 5 and 6 (e.g., wherein each X is the same), optionally wherein m is 4.

34. The compound or pharmaceutically acceptable salt thereof of any one of clauses 31-33, wherein X^{a} in each case is independently —(CH₂)ₜ— wherein each t is independently an integer selected from 2, 3 and 4 (e.g., wherein each X^{a} is the same), optionally wherein t is 3.

35. The compound or pharmaceutically acceptable salt thereof of any one of clauses 31-34, wherein the [tether]-[linker]-[cargo] moiety is selected from:
i)

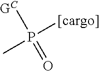

wherein G^{C} is Z (e.g., —OH);
ii)

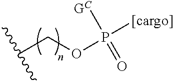

wherein n is selected from 3 and 4, and G^{C} is Z (e.g., —OH) or Y

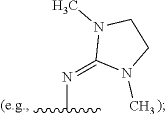 [SMILES: *N=C1N(C)CCN1C]

and
iii)

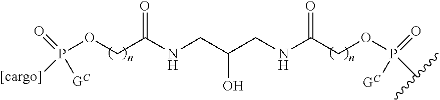

wherein n is 4, and G^{C} is Z (e.g., —OH).

36. A compound, or a pharmaceutically acceptable salt thereof, wherein the compound is of Formula (VII):

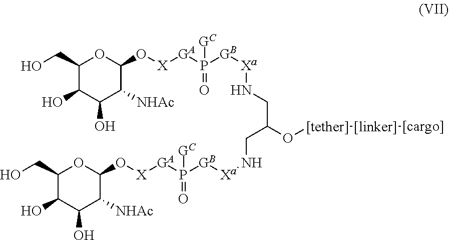

wherein X, G^{A} G^{B}, X^{a}, [tether], [linker] and [cargo] are as defined in any of the preceding clauses, and G^{C} in each case is independently selected from Y and Z as defined in any of the preceding clauses.

37. The compound or pharmaceutically acceptable salt thereof of clause 36, wherein G^{A} and G^{B} in each case are O, and wherein both of the G^{C} groups are independently selected from Y

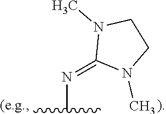 [SMILES: *N=C1N(C)CCN1C]

38. The compound or pharmaceutically acceptable salt thereof of clause 36 or clause 37, wherein X in each case is independently —(CH₂)ₘ— wherein each m is independently an integer selected from 3, 4, 5 and 6 (e.g., wherein each X is the same), optionally wherein m is 4.

39. The compound or pharmaceutically acceptable salt thereof of any one of clauses 36-38, wherein X^{a} in each case is independently —C(O)(CH₂)ᵤ—* (wherein * denotes the point of attachment to G^{B}), wherein u is an integer independently selected from 3, 4 and 5 and 6 (e.g., 4).

40. The compound or pharmaceutically acceptable salt thereof of any one of clauses 36-39, wherein the [tether]-[linker]-[cargo] moiety is

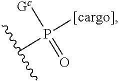

wherein G^{C} is Z (e.g., —OH).

41. A compound, or a pharmaceutically acceptable salt thereof, wherein the compound is of Formula (VIII):

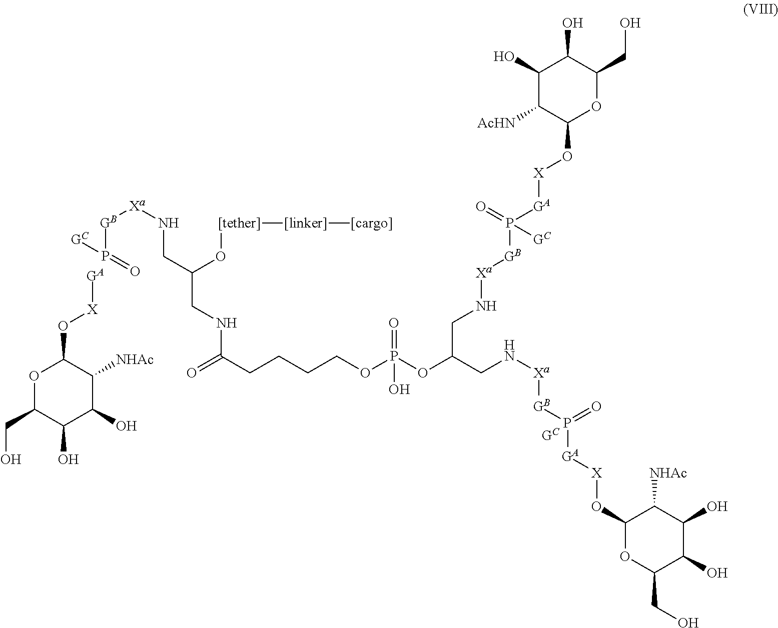

wherein, X, G^{A}, G^{B}, X^{a}, [tether], [linker] an [cargo] are as defined in any of the preceding clauses, and G^{C} in each case is independently selected from Y and Z as defined in any of the preceding clauses.

42. The compound or pharmaceutically acceptable salt thereof of clause 41, wherein G^{A} and G^{B} in each case are O, and wherein all three of the G^{C} groups are independently selected from Y

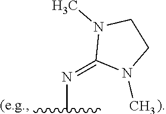 [SMILES: *N=C1N(C)CCN1C]

43. The compound or pharmaceutically acceptable salt thereof of clause 41 or clause 42, wherein X in each case is independently —(CH₂)ₘ— wherein each m is independently an integer selected from 3, 4, 5 and 6 (e.g., wherein each X is the same), optionally wherein m is 4.

44. The compound or pharmaceutically acceptable salt thereof of any one of clauses 41-43, wherein X^{a} in each case is independently —C(O)(CH₂)ᵤ—* (wherein * denotes the point of attachment to G^{B}), wherein u is an integer independently selected from 3, 4 and 5 and 6 (e.g., 4).

45. The compound or pharmaceutically acceptable salt thereof of any one of clauses 41-44, wherein the [tether]-[linker]-[cargo] moiety is

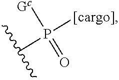

wherein G^{C} is Z (e.g., —OH).

46. A compound selected from:

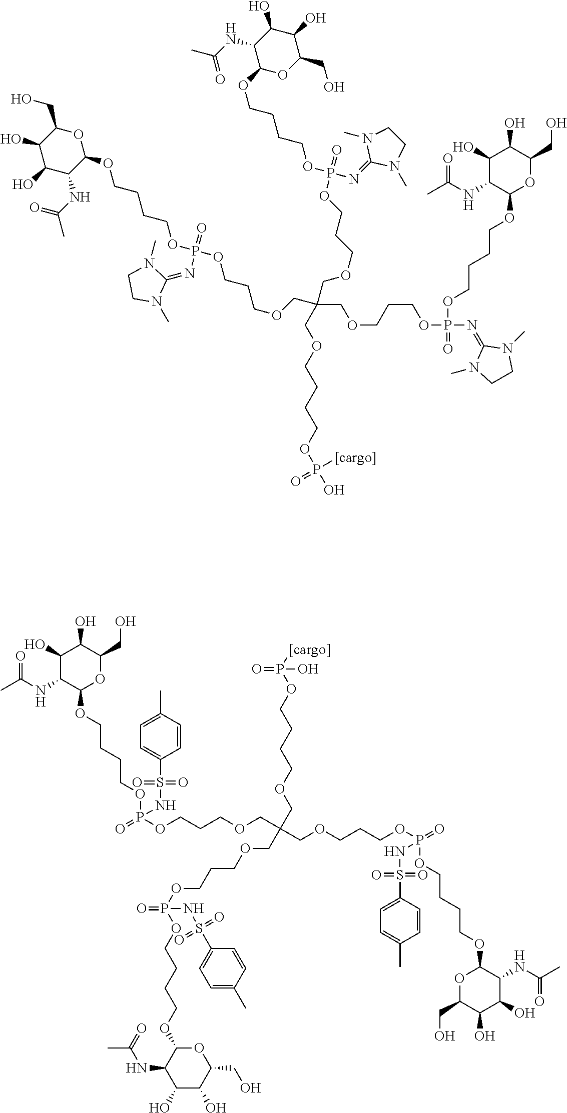

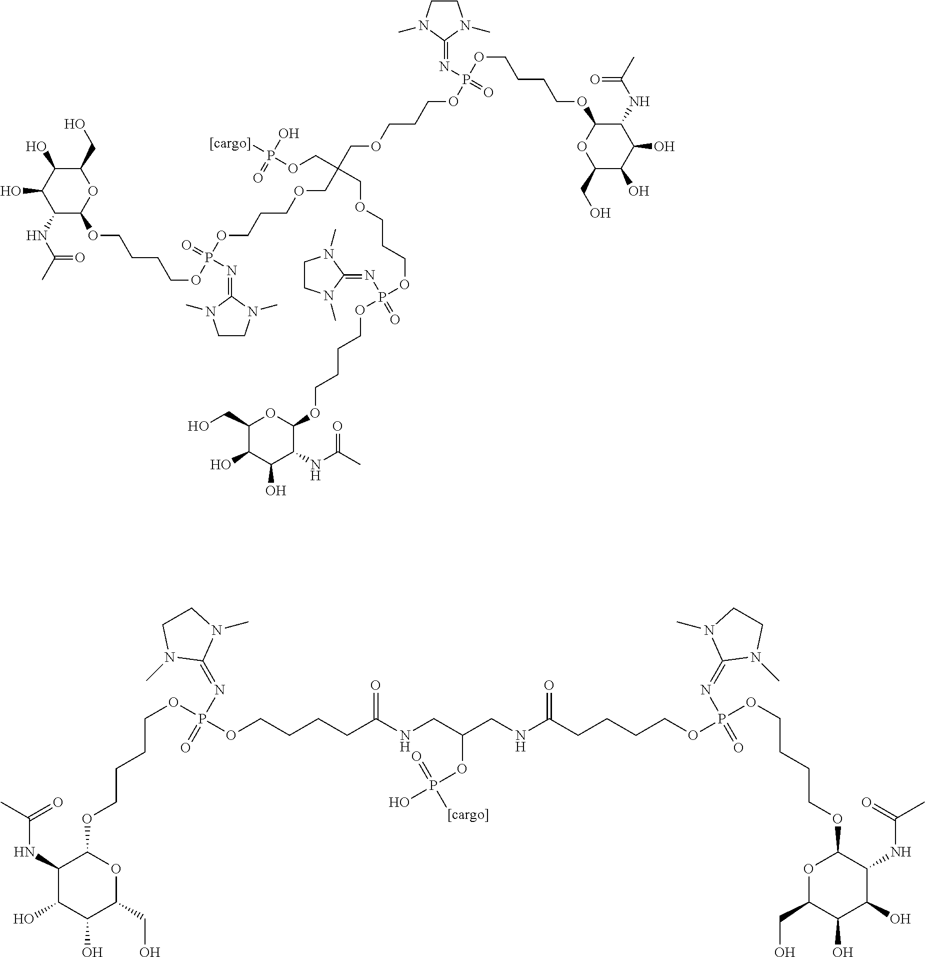

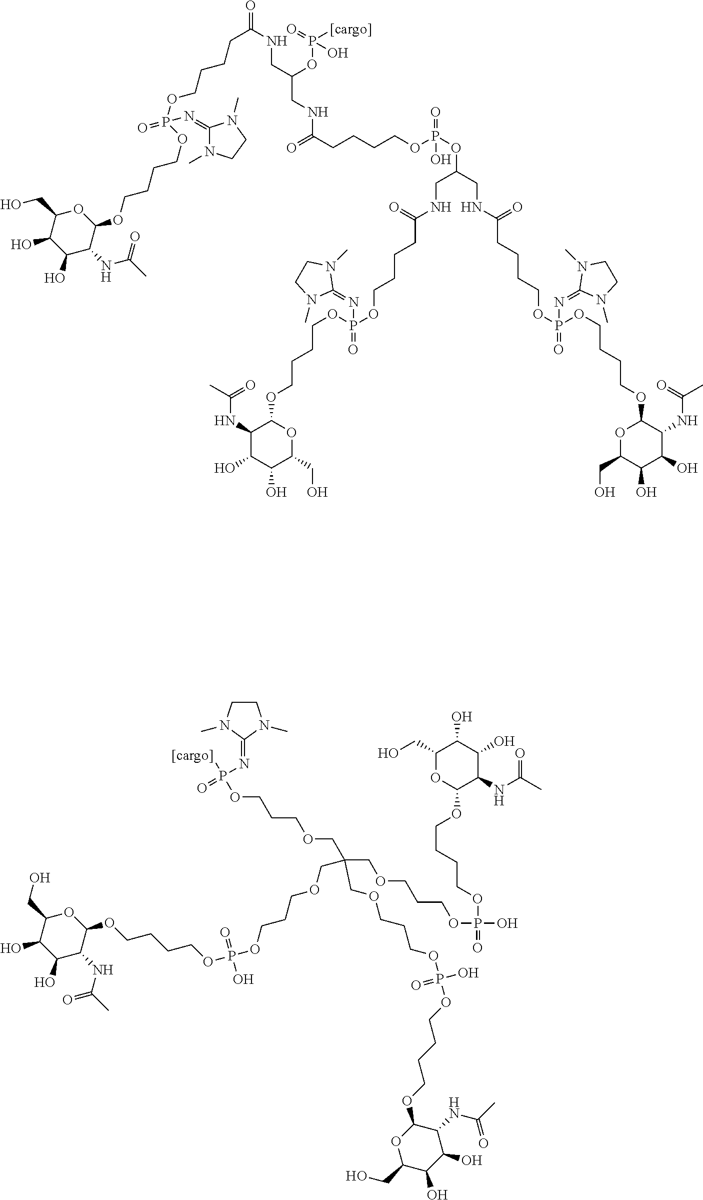

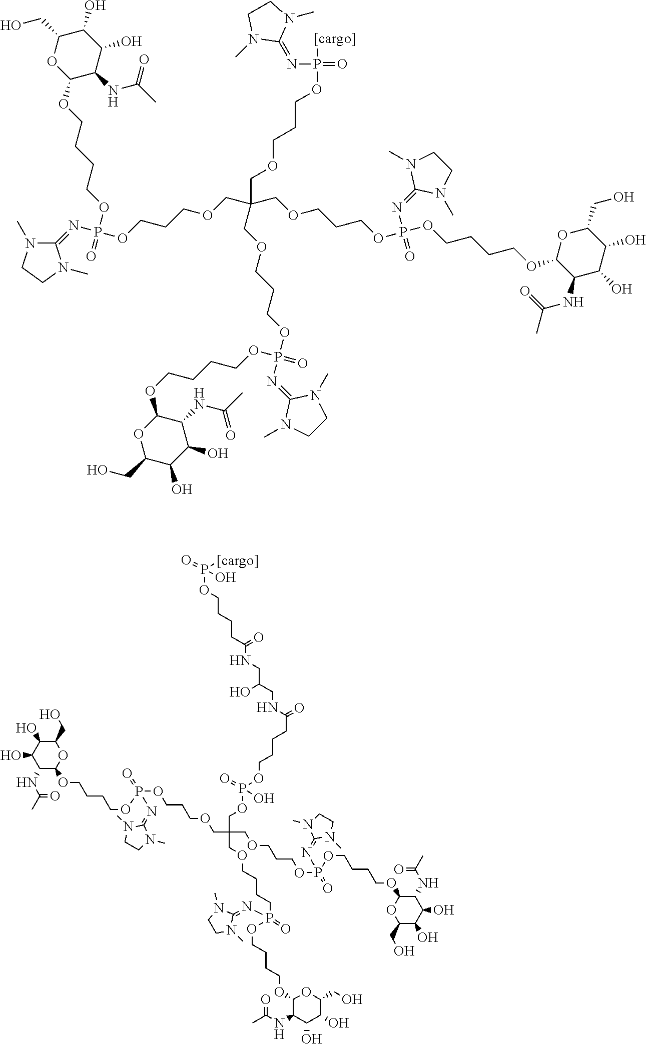

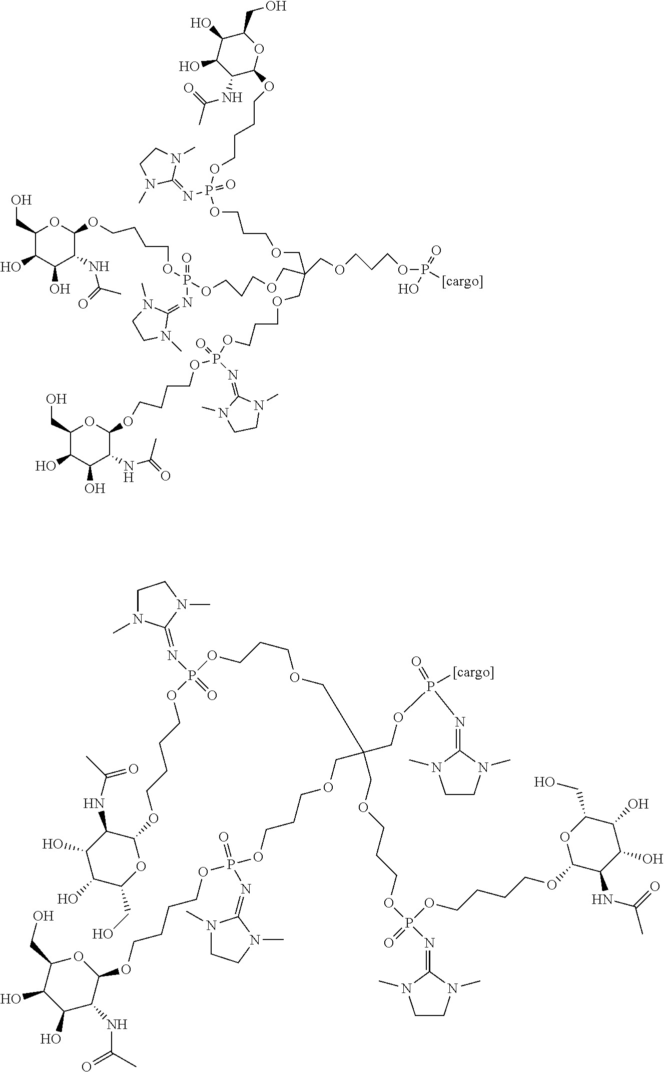

and the pharmaceutically acceptable salts thereof, wherein [cargo] denotes a cargo as defined in any of the preceding clauses.

47. A compound as defined in any one of clauses 1-46, or a pharmaceutically acceptable salt thereof, having an activity in knocking down gene expression in human primary hepatocytes which is characterised by an IC₅₀ value of less than about 2 μM (e.g., an IC₅₀ value of less than about 1.0 μM, 0.5 μM or 0.2 μM), optionally an IC₅₀ value of less than about 20 nM.

48. A compound as defined in any one of clauses 1-46, or a pharmaceutically acceptable salt thereof, having an activity in knocking down gene expression in HEK293 cells which is characterised by an IC₅₀ value of less than about 2 μM (e.g., an IC₅₀ value of less than about 1.5 μM, 1.0 μM, 0.5 μM or 0.2 μM), optionally an IC₅₀ value of less than about 75 nM or 50 nM.

49. A pharmaceutical composition comprising a compound as defined in any one of clauses 1-48, or a pharmaceutically acceptable salt thereof, and at least one pharmaceutically acceptable excipient or carrier.

50. A nucleic acid delivery agent or compound or pharmaceutically acceptable salt thereof according to any one of clauses 1-48, or a pharmaceutical composition according to clause 49, for use in therapy.

51. A nucleic acid delivery agent or compound or pharmaceutically acceptable salt thereof according to any one of clauses 1-48, or a pharmaceutical composition according to clause 49, for use in the treatment of a condition selected from liver disease (e.g., liver cancer), genetic disease, haemophilia and bleeding disorders, liver fibrosis, non-alcoholic steatohepatitis (NASH), non-alcoholic fatty liver disease (NAFLD), viral hepatitis, rare diseases, metabolic disease, cardiovascular disease, obesity, thalassemia, liver injury (e.g., drug induced liver injury), hemochromatosis, alcoholic liver disease, alcohol dependence, anaemia, and anaemia of chronic disease.

52. The nucleic acid delivery agent, compound, pharmaceutically acceptable salt or pharmaceutical composition for use of clause 51, wherein the condition is selected from NASH, NAFLD, a metabolic disease and a cardiovascular disease.

53. The nucleic acid delivery agent, compound, pharmaceutically acceptable salt or pharmaceutical composition for use of clause 51, wherein the condition is NASH.

54. The nucleic acid delivery agent, compound, pharmaceutically acceptable salt or pharmaceutical composition for use of clause 51, wherein the condition is a metabolic disease selected from hypercholesterolemia, dyslipidaemia, and hypertriglyceridemia.

### BRIEF DESCRIPTION OF THE FIGURES

FIG. 1 shows a schematic example of compounds of the disclosure. The cargo nucleic acid is attached to the rest of the molecule via a linker and tether. The splitter moiety is bound to the tether on one side and to multiple ligands on the other, in each case with a spacer in between the ligand and the splitter. The complex shown in the figure carries three ligands, but other numbers of ligands are possible (e.g., by employing different splitters). In operation, the ligands (which may be, e.g., GalNAc moieties) typically bind to a receptor or other biomolecule which is associated with a cell or tissue; this facilitates association with and/or uptake into the cell or tissue where the cargo (e.g., a siRNA) can exert a biological effect.

FIG. 2 shows results of the in vivo mouse knockdown study. Baseline ANGPTL3 protein levels are shown (FIG. 2A), with division of the dataset into Part A and Part B as described in Example 21. Part A data include time-course data of ANGPTL3 protein (FIG. 2B) and baseline and control-corrected data of ANGPTL3 protein (FIG. 2C). Part B data include time-course data of ANGPTL3 protein (FIG. 2D) and baseline and control-corrected data of ANGPTL3 protein (FIG. 2E). Relative expression of Angptl3 mRNA compared to vehicle in the liver on day 49 after dosing is also shown (FIG. 2F).

FIG. 3 shows results of the in vivo mouse knockdown study (Example 21). Relative expression of Malat1 mRNA as compared to vehicle at 24 h in the liver on 24 hours and 14 days after dosing is shown. Statistical analysis indicates two-way ANOVA with multiple comparison.

### DETAILED DESCRIPTION

Although specific embodiments of the present disclosure will now be described with reference to the description and examples, it should be understood that such embodiments are by way of example only and merely illustrative of but a small number of the many possible specific embodiments which can represent applications of the principles of the present disclosure. Various changes and modifications will be obvious to those of skill in the art given the benefit of the present disclosure and are deemed to be within the spirit and scope of the present disclosure as further defined in the appended claims.

### Definitions

Unless defined otherwise, all technical and scientific terms used herein have the same meanings as commonly understood by one of ordinary skill in the art to which this disclosure belongs. Although any methods and materials similar or equivalent to those described herein can be used in the practice or testing of the present disclosure, exemplary methods, devices, and materials are now described. All technical and patent publications cited herein are incorporated herein by reference in their entirety.

The practice of the present disclosure will employ, unless otherwise indicated, conventional techniques of chemical synthesis, tissue culture, immunology, molecular biology, microbiology, cell biology, recombinant DNA, etc., which are within the skill of the art. See, e.g., Michael R. Green and Joseph Sambrook, Molecular Cloning (4^{th} ed., Cold Spring Harbor Laboratory Press 2012); the series Ausubel et al. eds. (2007) Current Protocols in Molecular Biology; the series Methods in Enzymology (Academic Press, Inc., N.Y.); MacPherson et al. (1991) PCR 1: A Practical Approach (TRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5^{th} edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (TRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); Herzenberg et al. eds (1996) Weir's Handbook of Experimental Immunology; Manipulating the Mouse Embryo: A Laboratory Manual, 3^{rd} edition (Cold Spring Harbor Laboratory Press (2002)); Sohail (ed.) (2004) Gene Silencing by RNA Interference: Technology and Application (CRC Press).

All numerical designations, e.g., pH, temperature, time, concentration, molecular weight, etc., including ranges, are approximations which are varied (+) or (−) by increments of, e.g., 0.1 or 1.0, where appropriate. It is to be understood, although not always explicitly stated, that all numerical designations are preceded by the term “about”, which is used to denote a conventional level of variability. For example, a numerical designation which is “about” a given value may vary by ±10% of said value; alternatively, the variation may be ±5%, ±2%, or ±1% of the value. It also is to be understood, although not always explicitly stated, that the reagents described herein are merely exemplary and that equivalents of such are known in the art.

As used in the specification and claims, the singular forms “a”, “an”, and “the” include plural references unless the context clearly dictates otherwise. For example, the term “a cell” includes a plurality of cells, including mixtures thereof. Unless specifically stated or obvious from context, as used herein, the term “or” is understood to be inclusive. The term “including” is used herein to mean, and is used interchangeably with, the phrase “including but not limited to”.

As used herein, the term “comprising” or “comprises” is intended to mean that the compositions and methods include the recited elements, without excluding other elements. “Consisting essentially of” when used to define compositions and methods, shall mean excluding other elements of any essential significance for the stated purpose. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this disclosure or process steps to produce a composition or achieve an intended result. Embodiments defined by each of these transition terms are within the scope of this disclosure. Use of the term “comprising” herein is intended to encompass, and to disclose, the corresponding statements in which the term “comprising” is replaced by “consisting essentially of” or “consisting of”.

“GalNAc” refers to 2-(acetylamino)-2-deoxy-D-galactopyranose, commonly referred to in the literature as N-acetyl galactosamine. Reference to “GalNAc” or “N-acetyl galactosamine” herein may, in particular, denote the R form, 2-(acetylamino)-2-deoxy-β-D-galactopyranose.

The term “cargo” as used herein refers to a chemical or biological entity which is suitable for targeting to and/or delivery to a cell or tissue, e.g. as part of a compound of the disclosure. The cargo in accordance with the present disclosure is a nucleic acid.

The term “nucleic acid” as used herein includes nucleic acids selected from the group consisting of DNA, RNA, peptide nucleic acid (PNA), and locked nucleic acid (LNA). The nucleic acid may be a functional nucleic acid, e.g., whereby the functional nucleic acid is selected from the group consisting of mRNA, micro-RNA, shRNA, combinations of RNA and DNA, siRNA, siNA, antisense nucleic acid (e.g., antisense oligonucleotide (ASO)), ribozymes, aptamers and spiegelmers. A “peptide nucleic acid” is a polymer which is similar to DNA or RNA in which the backbone is composed of repeating amino acid (typically N-(2-aminoethyl)-glycine) units linked by peptide bonds. The various purine and pyrimidine bases are linked to the backbone by a methylene bridge and a carbonyl group. A “locked nucleic acid” is a nucleic acid in which the 2′ hydroxyl is connected, e.g., by a methylene bridge, to the 4′ carbon of the same ribose sugar.

By “duplex region” is meant the region in two complementary or substantially complementary oligonucleotides that form base pairs with one another, either by Watson-Crick base pairing or any other manner that allows for a duplex between oligonucleotide strands that are complementary or substantially complementary. For example, an oligonucleotide strand having 21 nucleotide units can base pair with another oligonucleotide of 21 nucleotide units, yet only 19 nucleotides on each strand are complementary or substantially complementary, such that the “duplex region” consists of 19 base pairs. The remaining base pairs may exist as 5′ and 3′ overhangs, or as single stranded regions. Further, within the duplex region, 100% complementarity is not required; substantial complementarity is allowable within a duplex region. Substantial complementarity refers to complementarity between the strands such that they are capable of annealing under biological conditions. Techniques to empirically determine if two strands are capable of annealing under biological conditions are well known in the art. Alternatively, two strands can be synthesised and added together under biological conditions to determine if they anneal to one another. The portion of the first strand and second strand that form at least one duplex region may be fully complementary and are at least partially complementary to each other.

The term “abasic” as used herein in connection with nucleotides, refers to moieties lacking a base or having other chemical groups in place of a base at the 1′ position, for example a 3′,3′-linked or 5′,5′-linked deoxyabasic ribose derivative.

The term “alternating” as used herein in connection with nucleic acids means to occur one after another in a regular way. In other words, alternating means to occur in turn repeatedly. For example if one nucleotide is modified, the next contiguous nucleotide is not modified and the following contiguous nucleotide is modified and so on.

As used herein, the term “inhibit”, “down-regulate”, or “reduce” with respect to gene expression means the expression of the gene, or level of RNA molecules or equivalent RNA molecules encoding one or more proteins or protein subunits (e.g., mRNA), or activity of one or more proteins or protein subunits, is reduced below that observed in the absence of a nucleic acid of the disclosure; for example the expression may be reduced to 90%, 80%, 70%, 60%, 50%, 40%, 30%, 20%, 10%, 5% or less than that observed in the absence of an inhibitor.

An “overhang” as used herein has its normal and customary meaning in the art, i.e. a single stranded portion of a nucleic acid that extends beyond the terminal nucleotide of a complementary strand in a double strand nucleic acid. The term “blunt end” includes double stranded nucleic acid whereby both strands terminate at the same position, regardless of whether the terminal nucleotide(s) are base paired.

A “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions of the disclosure is contemplated.

A “subject,” “individual”, or “patient” is used interchangeably herein, and typically refers to a vertebrate, such as a mammal. Mammals include, but are not limited to, rodents, farm animals, sport animals, pets, and primates; for example murines, rats, rabbit, simians, bovines, ovines, porcines, canines, felines, equines, and humans. In a particular embodiment, the mammal is a human.

“Administering” is defined herein as a means of providing an agent or a composition containing the agent to a subject in a manner that results in the agent being contacted with (e.g., being inside) the subject's body. Such an administration can be by any route including, without limitation, oral, transdermal (e.g., by the vagina, rectum, or oral mucosa), by injection (e.g., subcutaneous, intravenous, parenteral, intraperitoneal, or into the central nervous system), or by inhalation (e.g., oral or nasal). Administration may also involve providing a substance or composition to a part of the surface of the subject's body, for example by topical administration to the skin. Pharmaceutical preparations are, of course, given by forms suitable for each administration route.

“Treating” or “treatment” of a disease includes: (1) preventing the disease, i.e. causing the clinical symptoms of the disease not to develop in a patient that may be predisposed to the disease but does not yet experience or display symptoms of the disease; (2) inhibiting the disease, i.e. arresting or reducing the development of the disease or its clinical symptoms; and/or (3) relieving the disease, i.e. causing regression of the disease or its clinical symptoms. A patient or individual may be predisposed to the disease because of the presence of genetic mutations associated with the disease.

An “effective amount” or “therapeutically effective amount” is an amount sufficient to effect beneficial or desired results. An effective amount can be administered in one or more administrations, applications, or dosages. Such delivery is dependent on a number of variables including the time period for which the individual dosage unit is to be used, the bioavailability of the therapeutic agent, the route of administration, etc. It is understood, however, that specific dose levels of the therapeutic agents of the present disclosure for any particular subject depends upon a variety of factors including, for example, the activity of the specific compound employed, the age, body weight, general health, sex, and diet of the subject, the time of administration, the rate of excretion, the drug combination, the severity of the particular disorder being treated and the form of administration. Treatment dosages generally may be titrated to optimize safety and efficacy. Typically, dosage-effect relationships from in vitro and/or in vivo tests initially can provide useful guidance on the proper doses for patient administration. In general, one will desire to administer an amount of the compound that is effective to achieve a serum level commensurate with the concentrations found to be effective in vitro. Determination of these parameters is well within the skill of the art. These considerations, as well as effective formulations and administration procedures are well known in the art and are described in standard textbooks. Consistent with this definition, as used herein, the term “therapeutically effective amount” is an amount sufficient to treat (e.g., improve) one or more symptoms associated with the condition. The total daily dose may be administered in single or divided doses and may, at the physician's discretion, fall outside of the typical range given herein.

The term “delivering” when used in connection with the nucleic acid-containing compounds and compositions of the disclosure typically denotes some active targeting of the nucleic acid to the target cell or tissue. Thus, delivery of a nucleic acid using the compounds and compositions of the disclosure typically results in exposure of the target cell or tissue to the nucleic acid at a level which is greater than the exposure following administration of the same nucleic acid without the rest of the complex (e.g., when administered as ‘naked’ nucleic acid).

As used herein, the term “pharmaceutically acceptable excipient” or “pharmaceutically acceptable carrier” encompasses any of the standard pharmaceutical ingredients, for example as described in Remington's Pharmaceutical Sciences (20^{th} ed., Mack Publishing Co. 2000). Such excipients include carriers such as a phosphate buffered saline solution, water, and emulsions, such as an oil/water or water/oil emulsion, and various types of wetting agents. Pharmaceutical compositions also can include stabilizers, preservatives, adjuvants, fillers, binders, lubricants, and the like.

As used herein, the term “alkyl” means a saturated linear or branched free radical consisting essentially of carbon atoms and a corresponding number of hydrogen atoms. The term “alkylene” has the corresponding meaning in connection with the divalent free radical. Exemplary alkyl groups include methyl, ethyl, n-propyl, isopropyl, n-butyl, isobutyl, etc. Other alkyl groups will be readily apparent to those of skill in the art given the benefit of the present disclosure. The terms “(C₁-C₃)alkyl”, “(C₁-C₆)alkyl”, etc., have equivalent meanings, i.e., a saturated linear or branched free radical consisting essentially of 1 to 3 (or 1 to 6) carbon atoms and a corresponding number of hydrogen atoms. The definition of “alkyl” also applies in the context of other groups which comprise alkyl groups, such as “—O(C₁-C₃)alkyl”. The term “haloalkyl” means an alkyl group which is substituted by one or more halogens. Exemplary haloalkyl groups include trifluoromethyl, trifluoroethyl, difluoroethyl, pentafluoroethyl, chloromethyl, etc. One or more carbon atoms in the backbone of the alkyl group may be substituted by (or bonded to) a heteroatom by a multiple bond (e.g., a double bond); for example, a carbon atom of the alkyl group may be bonded to oxygen via a double bond (i.e., substituted by oxo to provide a carbonyl function). The presence of such a substituent does not prevent the carbon backbone of the free radical being considered as an alkyl group. In embodiments, the alkyl group is linear. Alkyl groups of the present disclosure may be substituted with one or more optional substituents as defined herein.

As used herein, the term “alkenyl” means an unsaturated linear or branched free radical consisting essentially of carbon atoms and a corresponding number of hydrogen atoms, which free radical comprises at least one carbon-carbon double bond. The term “alkenylene” has the corresponding meaning in connection with the divalent free radical. Exemplary alkenyl groups include ethenyl, prop-1-enyl, prop-2-enyl, isopropenyl, but-1-enyl, 2-methyl-prop-1-enyl, and 2-methyl-prop-2-enyl. The terms “(C₂-C₆)alkenyl”, etc., have equivalent meanings, i.e., an unsaturated linear or branched free radical consisting essentially of 2 to 6 carbon atoms and a corresponding number of hydrogen atoms. Alkenyl groups of the present disclosure may be substituted with one or more optional substituents as defined herein.

As used herein, the term “aryl” means an aromatic free radical having at least 6 carbon atoms (i.e., ring atoms) that form a ring. It will be appreciated that the aryl group may be monocyclic or multicyclic (e.g., fused). In the case of multicyclic aryl groups, there are further rings, e.g. 1 or more further rings, all of which contain at least 3 carbon atoms (i.e., ring atoms). Examples of aryl groups include phenyl and naphthalenyl. The aryl group may contain from 6 to 10 carbon atoms in the ring portion of the group, which may be monocyclic or multicyclic (e.g., fused). In embodiments, aryl is phenyl. Aryl groups of the present disclosure may be substituted with one or more optional substituents as defined herein.

As used herein, the term “cycloalkyl” means a saturated free radical having at least 3 to 9 carbon atoms (i.e., ring atoms) that form a ring. Exemplary cycloalkyl groups include cyclopropyl, cyclobutyl, cyclopentyl and cyclohexyl. It will be appreciated that the cycloalkyl group may be monocyclic or multicyclic (e.g., fused, bridged, or spirocyclic). In the case of multicyclic cycloalkyl groups, there are further rings, e.g. 1 or more further rings, all of which contain from 3 to 7 carbon atoms (i.e., ring atoms). Exemplary cycloalkyl groups having such further rings include bicyclo[1.1.1]pentanyl. One or more ring atoms of the cycloalkyl group may be substituted by (i.e., bonded to) a heteroatom by a double bond (e.g., cycloalkyl substituted by oxo). The presence of such a substituent does not prevent the carbon backbone of the free radical being considered as a cycloalkyl group. Cycloalkyl groups of the present disclosure may be substituted with one or more optional substituents as defined herein.

As used herein, the term “heterocycloalkyl” means a saturated free radical having at least 3 to 10 atoms (i.e., ring atoms) that form a ring, wherein at least 1 to 9 of said ring atoms are carbon and the remaining at least 1 to 9 ring atom(s) (i.e., hetero ring atom(s)) are selected independently from the group consisting of nitrogen, sulphur, and oxygen. Heterocycloalkyl rings may have oxo substituents, typically adjacent to a heteroatom (e.g., 2-oxopyrrolidinyl), but the oxygen atom does not form part of the ring and is excluded from the number of ring atoms. The presence of such a substituent does not prevent the ring (or rings) of the free radical being considered as a heterocycloalkyl group. Exemplary heterocycloalkyl groups include tetrahydrofuranyl, piperidinyl, morpholinyl and piperazinyl. In the case of multicyclic heterocyclic groups, there are further rings, e.g. 1 or more further rings, all of which contain from 3 to 7 ring atoms selected from carbon, nitrogen, sulphur, and oxygen. The further rings may be saturated, or partially or fully unsaturated (e.g., having aromatic character). Multicyclic heterocyclic groups include fused, bridged and spirocyclic ring systems. Where a multicyclic heterocycloalkyl group contains an unsaturated fused ring, the group is typically not bonded to the rest of the molecule via that fused ring. Heterocycloalkyl groups of the present disclosure may be substituted with one or more optional substituents as defined herein.

The term “phosphate” is typically used herein to denote a radical (or diradical) group which comprises a central phosphorus atom bonded to four oxygen atoms (one via a double bond). The oxygen atoms which are bonded to phosphorus via single bonds may represent points of attachment to the rest of the molecule, or may carry hydrogen atoms, or may be considered to carry a negative charge (e.g., in the case of salts). The term “thiophosphate” is typically used herein to denote a phosphate analogue in which at least one oxygen atom is replaced by sulphur; such groups are also called “phosphorothioates” herein. The term “phosphoramidate” is typically used herein to denote a phosphate analogue in which at least one oxygen atom is replaced by nitrogen; such groups are also called “phosphoroamidates” herein.

A “sulfonyl” group is a radical containing a sulphur atom in which the sulphur atom is bonded to two oxygen atoms via two double bonds. The sulfonyl group is typically attached to the rest of the molecule via said sulphur atom. Exemplary sulfonyl groups include alkylsulfonyl, in which the sulphur atom is also bonded to an alkyl group (e.g., mesyl, in which the alkyl group is methyl), and arylsulfonyl, in which the sulphur atom is also bonded to an aryl group (e.g., tosyl, in which the aryl group is p-toluenyl). A group other than oxygen which is attached to the sulphur atom (e.g., the alkyl or aryl group) may be substituted with one or more optional substituents as defined herein. A “sulfonamide” group is a radical in which an amine is bonded to the sulphur atom of a sulfonyl group; this may also be denoted as “—NH-sulfonyl” herein, i.e. where the nitrogen and sulphur atoms are bonded together. The terms “—NH-alkylsulfonyl” and “—NH-arylsulfonyl” are to be construed accordingly, i.e. where the nitrogen and sulphur atoms are bonded together.

The term “guanidine-containing” moiety, when used to define a part of a compound of the present disclosure, denotes a group comprising a central imine radical of general formula

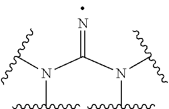

which is substituted by one or more hydrogen, alkyl, aryl, etc., groups. It will be appreciated that where hydrogen atoms are present on one or more of the nitrogen atoms shown above, the moiety may be written as one or more resonance forms of the above structure, such that the above structure is illustrative and not limiting. The various groups attached to the nitrogen atoms shown above may be separate (e.g., independently selected alkyl groups) or may be joined, in which case the guanidine-containing moiety is heterocyclic. Exemplary guanidine-containing moieties include 1,1,3,3-tetramethylguanidinyl and 1,3-dimethyl-2-(imino)imidazolidinyl.

An “optional substituent” is a group which is covalently attached to a moiety (generally via a carbon atom of the moiety, and typically in place of a hydrogen atom on said carbon atom). The optional substituent may be chosen to be a group which does not significantly alter the steric and/or electronic properties of the molecule. In embodiments, each optional substituent is independently selected from the group consisting of: halogen (e.g., —F, —Cl, —Br or —I); —OH; —SH; —NH₂; —NHMe; —NMe₂; —(C₁-C₃)alkyl (e.g., -Me or -Et); and 3- or 4-membered cycloalkyl or heterocycloalkyl group (e.g., cyclopropyl or epoxide), which may optionally be substituted with one or more halo. A group defined as “optionally substituted” may be either unsubstituted, or substituted with one or more substituents, e.g. 1, 2, 3, 4, 5, 6, or more substituents. In embodiments, a substituted group has 1 to 4 substituents, e.g. 1, 2, or 3 substituents. In embodiments, a substituted group has 1 or 2 substituents. In embodiments, a substituted group has 3 substituents.

As used herein, the terms “halo” and “halogen” mean fluorine, chlorine, bromine, or iodine. These terms are used interchangeably and may refer to a halogen free radical group or to a halogen atom as such. Those of skill in the art will readily be able to ascertain the identification of which in view of the context in which this term is used in the present disclosure. In embodiments, the halogen is fluorine.

The compounds of the present disclosure are described, inter alia, by way of structural formulae. It will be appreciated that these formulae typically show only one form (e.g., resonance form, tautomeric form, etc.) of the compound, whereas certain compounds may exist in more than one such form. This will be readily apparent to the skilled reader. The present disclosure includes all possible tautomers of the compounds characterised by the structural formulae herein, including as single tautomers, or as any mixture of tautomers in any ratio. It will also be appreciated that certain of the present compounds may exist in one or more isomeric (e.g., stereoisomeric) forms. The present disclosure includes all possible stereoisomers, enantiomers, diastereomers, etc. of the compounds described hereinbefore and below, as well as cis- and trans- forms and conformers of the same. The purification and the separation of isomers may be accomplished by methods described hereinafter, as well as by techniques known in the art. For example, optical isomers of the compounds can be obtained by resolution of the racemic mixture of diastereoisomeric salts thereof (e.g., using an optically active acid or base, or by the formation of covalent diastereomers). A different process for separation of optical isomers involves the use of chiral chromatography (e.g., HPLC columns using a chiral phase), with or without conventional derivatization. Enzymatic separation, with or without derivatisation, may also be useful, and optically active compounds of the present disclosure can likewise be obtained by chiral syntheses utilizing optically active starting materials. The present disclosure includes all possible stereoisomers of the compounds described herein as single stereoisomers, or as any mixture of said stereoisomers, e.g. (R)- or (S)- isomers, in any ratio.

The compounds of the disclosure may exist in the form of free acids or bases, or may exist as addition salts with suitable acids or bases. Methods for forming salts are described below and are also known in the art (see, e.g., Berge et al., J Pharm Sci. (1977) 66:1-19). As used herein, the term “pharmaceutically acceptable” when used in connection with salts means a salt of a currently disclosed compound that may be administered without any resultant substantial undesirable biological effect(s) or any resultant deleterious interaction(s) with any other component of a pharmaceutical composition in which it may be contained.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Compounds, compositions and methods provided herein may be combined with one or more of any of the other compounds, compositions and methods provided herein.

The following abbreviations and empirical formulae are used herein:
Ac acetyl
ACN acetonitrile
ANGPTL3 angiopoietin-like 3
anti-miR anti-microRNA
Appp 5′-adenosine cap
ASGPR asialoglycoprotein receptor
ASO antisense oligonucleotide
cDNA complementary DNA
cEt constrained ethyl
CR desired median normalized value for the central reference (neutral)
CRISPR clustered regularly interspaced short palindromic repeats
dA deoxyadenosine
dC deoxycytidine
DCM dichloromethane
dG 2′-deoxyguanosine
DMEM Dulbecco's modified eagle medium
DMTr 4,4′-dimethoxytrityl
DNA deoxyribonucleic acid
Ds 7-(2-thienyl)-imidazo[4,5-b]pyridine
dT thymidine
ELISA enzyme-linked immunosorbent assay
ESI electrospray ionization
GalNAc 2-(acetylamino)-2-deoxy-D-galactopyranose
HEK293 human embryonic kidney 293
HPLC high performance liquid chromatography
invAb inverted abasic deoxyribose
LCMS liquid chromatography mass spectrometry
LNA locked nucleic acid
m/z mass to charge ratio
m6A N6-methyladenosine
MALAT1 metastasis associated lung adenocarcinoma transcript 1
mC 5′-methylcytidine
MEM minimal essential medium
MMT 5′-monomethoxytrityl
MOE methoxyethyl
MPEG poly(ethylene glycol) methyl ether
MPS mononuclear phagocyte system
mQ Milli-Q®
mRNA messenger RNA
MS mass spectrometry
NAFLD non-alcoholic fatty liver disease
NASH non-alcoholic steatohepatitis
Pa pyrrole 2-carbaldehyde
PBS phosphate-buffered saline
PCR polymerase chain reaction
PEG polyethyleneglycol
PG/PGua phosphoryl guanidine
PNA peptide nucleic acid
PNS N-sulfonylphosphoramidate
PO phosphate
pol III RNA polymerase III
PPIB peptidyl-prolyl cis-trans isomerase B
PS thiophosphate
qPCR real-time polymerase chain reaction
RES reticuloendothelial system
RISC RNA-induced silencing complex
RNA ribonucleic acid
RNAi RNA interference
saRNA small activating ribonucleic acid
SEQ ID sequence identifier
shRNA short hairpin RNA
siNA small interfering nucleic acid
siRNA small interfering RNA
SR desired median normalized value for the scale reference (stimulator)
TAMRA carboxytetramethylrhodamine
TBAA tetrabutylammonium acetate
THE tetrahydrofuran
TOF time of flight
Tos toluenesulfonyl
TPPC4 trafficking protein particle complex subunit 4
U uracil
UV ultraviolet

### Compounds

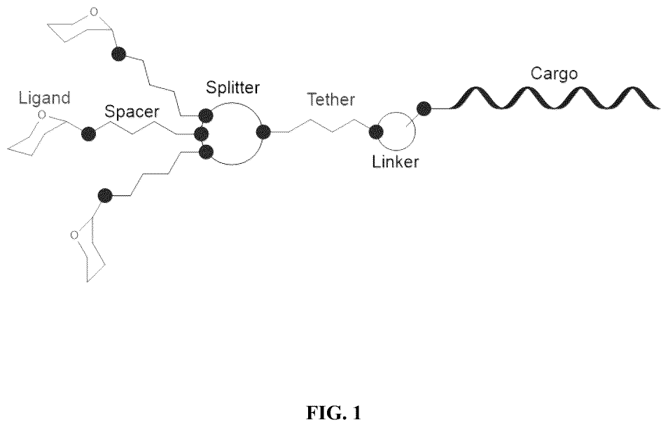

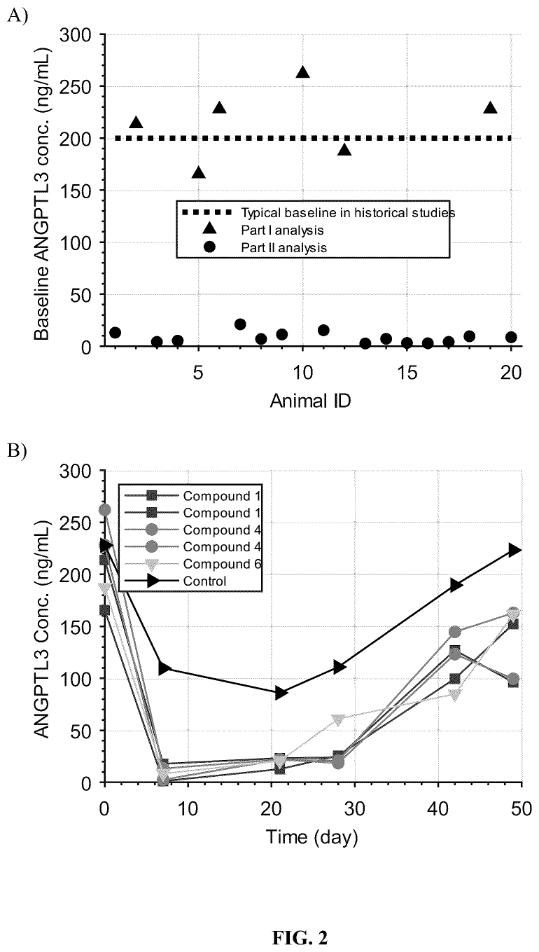

The present disclosure provides monosaccharide-containing compounds and complexes which are useful, inter alia, for targeting nucleic acid cargo moieties such as therapeutic oligonucleotides to specific locations (e.g., cell and/or tissue types) in vivo. As illustrated schematically in FIG. 1, the compounds have several ‘arms’ which carry the targeting ligands and cargo moieties. These, in turn, are typically bound to one or more central ‘splitter’ moieties (also called “branching units”) via a chain of atoms which serves as a ‘linker’ or ‘spacer’. The present disclosure relates, in particular, to compounds in which the spacer (or linker) comprises modified phosphate groups, especially having one or more phosphoramidate or substituted phosphoramidate groups.

Thus, in a first aspect the present disclosure provides a compound, or a pharmaceutically acceptable salt thereof, wherein the compound comprises at least one moiety having the structure of Formula (I):

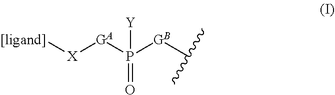

wherein:
X is selected from optionally substituted —(C₁-C₁₀)alkylene-, —(C₁-C₁₀)alkenylene- and *—[(CH₂)₂O]ₒ(CH₂)₂— (wherein * denotes the point of attachment of the group to G^{A}, and wherein o is an integer selected from 1, 2, and 3);
G^{A} and G^{B} are each independently selected from 0 and NH, provided that at least one of G^{A} and G^{B} is O;
Y is an —NH-sulfonyl group or a guanidine-containing moiety; and
[ligand] denotes a monosaccharide.

The compound may be a multivalent, e.g. branched, complex which comprises one or more cargo moieties as defined herein (e.g., one or more therapeutic nucleic acids). For example, the compound may be a nucleic acid delivery agent (e.g., an RNA delivery agent) which is a compound, or a pharmaceutically acceptable salt thereof, wherein the compound comprises a nucleic acid and at least one moiety having the structure of Formula (I). Furthermore, the ligand(s) may be chosen such that the compound has an affinity for ASGPR, e.g. having binding properties and/or an activity as defined herein. In embodiments, the compound is for delivering nucleic acid(s) to cells and/or tissue which express ASGPR.

In embodiments, X is selected from optionally substituted —(C₃-C₆)alkylene- and *—[(CH₂)₂O]ₒ(CH₂)₂— (wherein * denotes the point of attachment of the group to G^{A}, and wherein o is an integer selected from 1, 2, and 3). In embodiments, X is unsubstituted.

In embodiments, X is the alkylene group —(CH₂)ₘ— wherein m is an integer selected from 3, 4, 5 and 6 (e.g., 3, 4 or 5). In embodiments, m is 3 or 4. In embodiments, m is 4 or 5. In embodiments, m is 3. In embodiments, m is 4. In embodiments, m is 5. In other embodiments, X is —CH₂CH₂OCH₂CH₂—.

In embodiments, G^{A} and G^{B} are each independently O. In other embodiments, G^{A} is O and G^{B} is NH. In other embodiments, G^{A} is NH and G^{B} is O.

In embodiments, Y is an —NH-sulfonyl group. In embodiments, Y is an —NH-alkylsulfonyl group or an —NH-arylsulfonyl group, wherein alkyl denotes optionally substituted —(C₁-C₆)alkyl and aryl denotes optionally substituted phenyl. In embodiments, Y is selected from —NH-mesyl, —NH-phenylsulfonyl and —NH-tosyl. In embodiments, Y, G^{A} and G^{B}, together with the adjacent P=O group, form an N-sulfonylphosphoramidate group. In embodiments, the N-sulfonylphosphoramidate group is selected from:

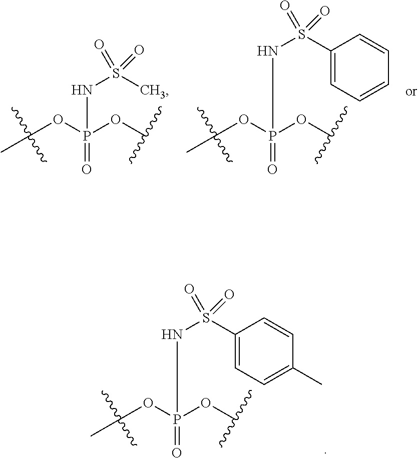 [SMILES: *OP(=O)(NS(C)(=O)=O)O* ; *OP(=O)(NS(=O)(=O)c1ccccc1)O* ; *OP(=O)(NS(=O)(=O)c1ccc(C)cc1)O*]

In other embodiments, Y is a guanidine-containing moiety. In embodiments, Y is a 1,3-dimethyl-2-(imino)imidazolidine group which is bonded to phosphorus via the imino nitrogen atom. In embodiments, Y, G^{A} and G^{B}, together with the adjacent P=O group, form a phosphoryl guanidine group. In embodiments, the phosphoryl guanidine group is:

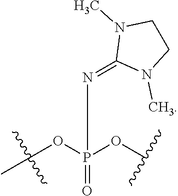 [SMILES: *OP(=O)(N=C1N(C)CCN1C)O*]

In embodiments, the ligand binds to ASGPR, e.g., the ligand is N-acetyl galactosamine (GalNAc). Viewed from this aspect the disclosure provides a compound, or a pharmaceutically acceptable salt thereof, wherein the compound comprises at least one moiety having the structure of Formula (II):

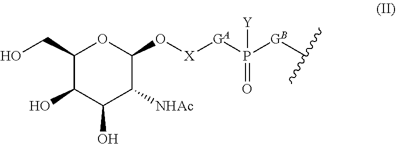

wherein X, G^{A}, G^{B}, and Y are as defined herein. In embodiments:
X is selected from optionally substituted —(C₃-C₆)alkylene-, and *—[(CH₂)₂O]ₒ(CH₂)₂— (wherein * denotes the point of attachment of the group to G^{A}),
wherein o is 1, 2 or 3;
G^{A} and G^{B} are each independently selected from O and NH, provided that at least one of G^{A} and G^{B} is O; and
   Y is an —NH-alkylsulfonyl group, an —NH-arylsulfonyl group, or a guanidine-containing moiety.

The compound may be a multivalent, e.g. branched, complex which comprises one or more cargo moieties as defined herein (e.g., one or more therapeutic nucleic acids). For example, the compound may be a nucleic acid delivery agent (e.g., an RNA delivery agent) which is a compound, or a pharmaceutically acceptable salt thereof, wherein the compound comprises a nucleic acid and at least one moiety having the structure of Formula (II). In embodiments, the compound is for delivering nucleic acid(s) to cells and/or tissue which express ASGPR.

In embodiments, X is the alkylene group —(CH₂)ₘ—, wherein m is an integer selected from 3, 4, 5 and 6 (e.g., 3, 4 or 5). In embodiments, m is 3 or 4. In embodiments, m is 4 or 5. In embodiments, m is 3. In embodiments, m is 4. In embodiments, m is 5. In other embodiments, X is —CH₂CH₂OCH₂CH₂—.

In embodiments, G^{A} and G^{B} are each independently O. In other embodiments, G^{A} is O and G^{B} is NH. In other embodiments, G^{A} is NH and G^{B} is O.

In embodiments, Y is selected from:

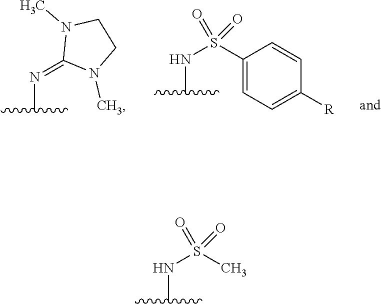

wherein R is —CH₃ or —H.

In embodiments, Y is

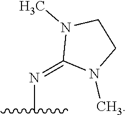 [SMILES: *N=C1N(C)CCN1C]

In other embodiments, Y is

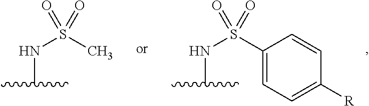

wherein R is —CH₃ or —H. In embodiments, Y is

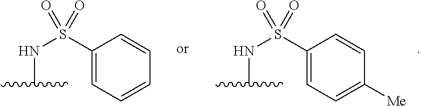 [SMILES: *NS(=O)(=O)c1ccccc1 ; *NS(=O)(=O)c1ccc(C)cc1]

In embodiments, Y is

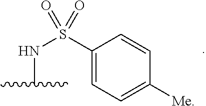 [SMILES: *NS(=O)(=O)c1ccc(C)cc1]

In other embodiments, Y is

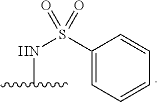 [SMILES: *NS(=O)(=O)c1ccccc1]

In embodiments, Y is

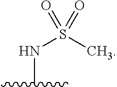 [SMILES: *NS(C)(=O)=O]

The structure of Formula (I) carries a ligand which may facilitate or promote binding of the compound to ASGPRs. The ligand in the structure of Formula (II) is a GalNAc moiety. The compounds (e.g., complexes) of the disclosure may thus be useful for the targeted delivery of nucleic acids to cells and tissues. As such, the compounds (e.g., complexes) of the disclosure may comprise a branched structure which carries one or more moieties of Formula (I) and/or Formula (II) and at least one cargo moiety.

Viewed from this aspect the disclosure provides a compound, or a pharmaceutically acceptable salt thereof, wherein the compound comprises: one or more (e.g., two or three) moieties having a structure independently selected from Formula (I) and Formula (II) as defined herein; and one or more (e.g., one) cargo moiety. The structure of this compound is illustrated by Formula (III):

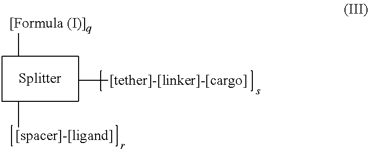

wherein q is an integer selected from 1, 2, 3 and 4; r is an integer selected from 0, 1, 2 and 3; and s is an integer selected from 1 and 2. The sum of q, r and s is typically 3, 4 or 5, i.e. the compound typically carries more than two and fewer than six arms. Each “Formula (I)” group has a structure independently selected from Formula (I) as defined herein. In embodiments, each “Formula (I)” group has a structure independently selected from Formula (II) as defined herein. In embodiments, any “ligand” groups which are present are GalNAc groups, e.g., such that the molecule exclusively carries GalNAc ligands.

The “spacer”, “ligand”, “tether”, “linker” and “cargo” groups are each independently as defined herein (and are illustrated in, e.g., FIG. 1). Thus, for example, the “ligand” is a monosaccharide target binding moiety as described herein.

Each ligand which is present in the compounds of the disclosure is a monosaccharide. The monosaccharide may be selected to have an affinity for the hepatic asialoglycoprotein receptor (ASGPR). In embodiments, the target ASGPR is on the surface of a mammalian liver cell. In embodiments, the ligand is GalNAc. In embodiments, the compounds of the disclosure have 2, 3 or 4 (e.g., 2 or 3) ligands which are all GalNAc.

The “tether” and “linker” groups taken together act to join the cargo moiety (or moieties) to the rest of the molecule, i.e. to the splitter. Suitable tethers and linkers will be apparent to the skilled person based on the current description and its Examples. In embodiments, the [tether]-[linker] moiety is obtained by reacting a tether precursor (which may be bonded to the rest of the molecule, i.e. to the spacer/ligand construct) with a linker precursor (which may be bonded to the cargo) to join the two together. Suitable methods for reacting the tether and linker precursors are described in the following Examples and would be known to the skilled person. Examples of such methods include ‘click’ chemistry reactions such as copper catalysed cycloadditions between an azide and an alkyne (see, e.g., Fantoni et al., Chem. Rev. (2021) 121(12):7122-7154). In other embodiments, the [tether]-[linker] moiety is introduced into the complex without the cargo attached and the cargo is subsequently bound to the linker. The complex may conveniently be coupled with a cargo in a final step of a solid-phase synthesis, e.g. where the [tether]-[linker] moiety is initially introduced with a phosphate group at its distal end that can be reacted with a cargo nucleic acid on solid phase to yield the final compound (see, e.g., Beaucage, Curr Opin Drug Discov Dev (2008) 11(2):203-216). Other examples of tethers and/or linkers which might be used in accordance with the present disclosure are found, e.g., in international patent publications WO 2014/179620, WO 2015/177668, WO 2009/073809, WO 2012/083046, WO 2017/156012, WO 2016/100401, WO 2017/174657 and WO 2019/092280, the contents of each of which are incorporated herein in their entirety. In embodiments, the tether and linker together represent a moiety comprising a linear chain of 8 to 30 atoms which links the cargo to the rest of the molecule. In embodiments, the tether and linker together represent an optionally substituted linear chain of 8 to 30 atoms selected from C, N, O, S and P, e.g. a chain of 9 to 24 atoms, 10 to 20 atoms, or 12 to 18 atoms. In embodiments, the tether and linker together comprise one or more groups selected from an amide (e.g., obtained by reaction of a carboxylic acid or acid derivative with an amine), an ester (e.g., obtained by reaction of a carboxylic acid or acid derivative with an alcohol, such as an aliphatic or aromatic alcohol), or a 1,2,3-triazole (e.g., obtained by reaction of an azide with an alkyne). In embodiments, the tether and linker together comprise (e.g., consist of) a group represented by:

—(CH₂)ₐ-D-(CH₂)_{b}—* or —C(O)(CH₂)_{c}-D-(CH₂)_{b}—*

wherein a is an integer from 0 to 12 (e.g., from 2 to 8, such as 6); D is selected from (i) a direct bond, (ii) —C(O)NH—, and (ii) a 1,2,3-triazole containing group; b is an integer from 0 to 12 (e.g., from 2 to 8, such as 6); and c is an integer from 0 to 10 (e.g., from 1 to 7. such as 4). The group may be optionally substituted and the * denotes the point of attachment to the cargo. It will be appreciated that the [tether]-[linker] group illustrated above will typically be connected to the cargo via a phosphate group or derivative thereof. Thus, in embodiments tether and linker together comprise a group as shown above which further comprises a phosphate (PO), thiophosphate (PS), phosphoryl guanidine (PG), or N-sulfonylphosphoramidate group (e.g., a group as defined herein) at the position marked with *.

In embodiments where the tether and linker together comprise (e.g., consist of) a group represented by —(CH₂)ₐ-D-(CH₂)_{b}—*, D may be a direct bond and b may be 0. The tether and linker together may thus comprise (e.g., consist of) a group represented by —(CH₂)ₐ—*, wherein a is an integer from 0 to 12. In embodiments, a is an integer from 2 to 8, e.g. 3 or 4. In embodiments, the tether and linker together comprise (e.g., consist of) —(CH₂)₄—*, and further comprise a phosphoryl guanidine (PG) or N-sulfonylphosphoramidate group (e.g., a group as defined herein) at the position marked with *. In other embodiments, a is 0, in which case the tether and linker together comprise (e.g., consist of) a direct bond; in this embodiment, a heteroatom of the splitter is typically bonded directly to the phosphorus atom of the phosphate group (or the derivative thereof).

In other embodiments, D is —C(O)NH—. The tether and linker together may thus comprise (e.g., consist of) a group represented by —(CH₂)ₐ—C(O)NH—(CH₂)_{b}—*, wherein a is an integer from 1 to 12 (e.g., 6), and b is an integer from 0 to 12 (e.g., 6). In embodiments, a is 6 and b is 6.

In embodiments where the tether and linker together comprise (e.g., consist of) a group represented by —C(O)(CH₂)_{c}-D-(CH₂)_{b}—*, D may be a direct bond and b may be 0. The tether and linker together may thus comprise (e.g., consist of) a group represented by —C(O)(CH₂)_{c}—*, wherein c is an integer from 0 to 10. In embodiments, c is an integer from 2 to 8, e.g. 4.

In yet further embodiments, D is a 1,2,3-triazole containing group, e.g. a group which is obtained by the reaction of BCN with an azide. Thus, in embodiments D comprises (e.g., consists of) a group

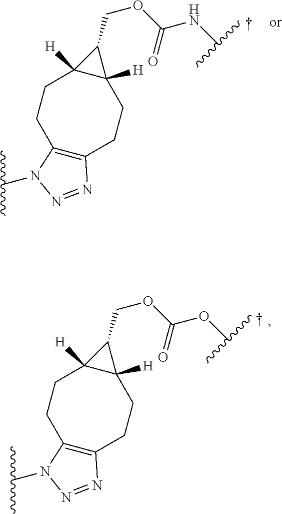 [SMILES: *NC(=O)OC[C@@H]1[C@@H]2CCc3nnn(*)c3CC[C@@H]21 ; *OC(=O)OC[C@@H]1[C@@H]2CCc3nnn(*)c3CC[C@@H]21]

wherein † denotes the point of attachment to —(CH₂)_{b}—. In embodiments, tether and linker together comprise (e.g., consist of) a group represented by

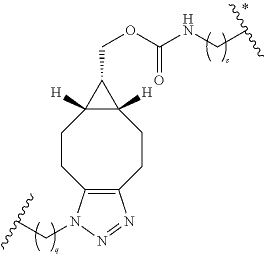

wherein a and b are defined herein (e.g., wherein a is 6 and b is 6).

As will be appreciated, the [tether]-[linker] moiety can conceptually be ‘split’ in a number of ways to yield the individual tether and linker components. In embodiments, the [tether]-[linker] moiety is generated by the reaction of a cargo-containing moiety (that comprises the linker, or a portion thereof) with a ligand-containing precursor (that comprises the tether, or a portion thereof). Thus, in embodiments the tether is represented by a group —(CH₂)ₐ— or —(CH₂)ₐ—C(O)—, wherein a is as defined herein. Likewise, in embodiments the linker is represented by a group -D-(CH₂)_{b}—* or —NH—(CH₂)_{b}—*, wherein D and b are as defined herein. In embodiments where tether and linker together denote an alkylene group attached to the cargo via a phosphate group (or a derivative thereof), the tether may be considered to be the alkylene group and the linker may be considered to be the phosphate group (or a derivative thereof). In embodiments where tether and linker together denote a phosphate group (or a derivative thereof) directly bonded to the splitter, the tether may be considered to be a bond and the linker may be considered to be the atoms in the phosphate group (or a derivative thereof) except for the heteroatom of the splitter.

The “spacer” comprises a chain of atoms, typically carbon atoms with one or more heteroatoms (e.g. selected independently from N, O, S, and P) optionally intervening that serves to link the ligand to the splitter moiety. In embodiments, the spacer comprises a chain of 2-20 atoms selected from C, N, O, S and P (e.g., a chain of 7-14 atoms). Exemplary spacers include linear alkylenes (which may optionally be interrupted by one or more amide and/or phosphate groups) and polyethylene glycols. Suitable spacers will be apparent to the skilled person based on the current description and its Examples. Other examples of spacers which might be used in accordance with the present disclosure are found, e.g., in international patent publications WO 2014/179620, WO 2015/177668, WO 2009/073809, WO 2012/083046, WO 2017/156012, WO 2016/100401, WO 2017/174657 and WO 2019/092280, the contents of each of which are incorporated herein in their entirety.

In embodiments, the spacer has a structure which comprises (e.g., which is):

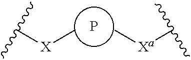

wherein: X is as defined herein; X^{a} is optionally substituted —(CH₂)ₜ— or —C(O)(CH₂)ᵤ—* (wherein * denotes the point of attachment to the group “P”), wherein t and u are integers independently selected from 1, 2, 3, 4, 5 and 6 (e.g., 2, 3, 4 or 5); and group “P” is selected from phosphate, thiophosphate, and phosphoramidate. In embodiments, X is —(CH₂)ₘ—, or *—[(CH₂)₂O]ₒ(CH₂)₂— (wherein * denotes the point of attachment of the group to “P”), wherein m is an integer selected from 3, 4 and 5, and o is an integer selected from 1, 2 and 3. Where the spacer has the above structure, X is typically attached to the [ligand] and X^{a} to the splitter (e.g, directly bonded). In embodiments, X is —(CH₂)₄—, and X^{a} is —(CH₂)₃— (wherein X is directly bonded to the [ligand], e.g. directly bonded to an oxygen atom of a GalNAc, and X^{a} is directly bonded to the splitter). In embodiments, X is —(CH₂)₄—, X^{a} is —(CH₂)₃—, and group “P” is phosphoramidate.

The “cargo” groups of the compounds disclosed herein are nucleic acids. These may be single stranded or double stranded. In embodiments, the cargo is an oligonucleotide. In embodiments, the cargo is selected from an antisense oligonucleotide (ASO), an immunostimulatory oligonucleotide, a decoy oligonucleotide, a splice altering oligonucleotide, a splice-switching oligonucleotide, a triplex forming oligonucleotide, a siRNA, a saRNA, a microRNA, a microRNA mimic, an anti-miR, a double stranded RNA, a single stranded RNA, a ribozyme, an aptamer, a spiegelmer, a CRISPR oligonucleotide and a G-quadruplex. In one embodiment, the cargo is an antisense oligonucleotide (ASO). In another embodiment, the cargo is a siRNA.

The “splitter” is a moiety having at least 2 (e.g., 3, 4 or 5, and typically 4) points of attachment for the ‘arms’ of the complex. It is typically derived from a precursor which has multiple reactive groups which may be the same or different. Suitable splitters will be apparent to the skilled person based on the current description and its Examples. Other examples of splitters which might be used in accordance with the present disclosure are found, e.g., in international patent publications WO 2014/179620, WO 2015/177668, WO 2009/073809, WO 2012/083046, WO 2017/156012, WO 2016/100401, WO 2017/174657 and WO 2019/092280, the contents of each of which are incorporated herein in their entirety.

In embodiments, the splitter is (or comprises) the group:

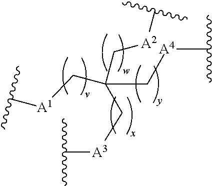

wherein: A¹, A², A³ and A⁴ are each independently selected from —O—, —S—, —NH—, —CH₂—, and —C(O)—; and v, w, x and y are each an integer independently selected from 0, 1, 2 and 3.

In embodiments, each of A¹, A², A³ and A⁴ is —O—. In other embodiments, each of A¹, A² and A³ is —O—, and A⁴ is —NH—. In embodiments, each of v, w, x and y is 1. In other embodiments, each of v, w and x is 1, and y is 0. In embodiments, each of A¹, A², A³ and A⁴ is —O—, and each of v, w, x and y is 1. In other embodiments, each of A¹, A² and A³ is —O—, A⁴ is —NH—, each of v, w and x is 1, and y is 0. In embodiments, the [Formula (I)] and [spacer]-[ligand] moieties are connected to the splitter via A¹, A² and A³, and a [tether]-[linker]-[cargo] moiety is connected to the splitter via A⁴.

In embodiments, the compound carries two, three or four (e.g., two or three) ligand-containing groups and one or two cargo-containing groups, wherein at least one of the ligand-containing groups does not have a structure of Formula (I) as defined herein. In this embodiment, the ligand-containing groups can comprise a phosphate and/or a phosphorothioate group in place of the phosphoroamidate. Including one or more phosphate and/or phosphorothioate groups in the compound of the disclosure can help to tailor the properties of the compound, e.g., pharmacokinetic properties such as plasma stability.

Viewed from this aspect the disclosure provides a compound, or a pharmaceutically acceptable salt thereof, having a structure as illustrated by Formula (IV):

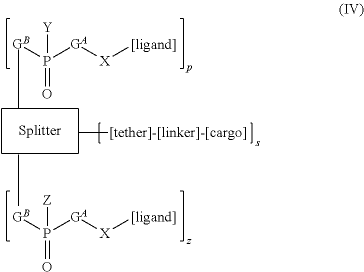

wherein X, Y, G^{A}, G^{B}, s, Splitter, [ligand], [tether], [linker] and [cargo] are as defined herein; p and z are each independently an integer selected from 0, 1, 2, 3 and 4; and Z in each case is independently selected from —SH and —OH, and wherein the sum of p and z is 2, 3 or 4, with the proviso that when p is 0, at least one [tether]-[linker] moiety comprises a phosphoryl guanidine or N-sulfonylphosphoramidate group (e.g., a group as defined herein).

In embodiments:
X in each case is independently optionally substituted —(CH₂)ₘ— or *—[(CH₂)₂O]ₒ(CH₂)₂— (wherein * denotes the point of attachment of the group to G^{A}), wherein
   each m is independently an integer selected from 3, 4, 5 and 6, and
   each o is independently an integer selected from 1, 2 and 3;
G^{A} and G^{B} in each case are independently selected from 0 and NH, provided that at least one of the G^{A} and G^{B} attached to the same P atom is O in each case;
Y in each case is independently an —NH-sulfonyl group or a guanidine-containing moiety (e.g., as defined herein);
Z in each case is independently selected from —SH and —OH;
p and z are each independently integers selected from 0, 1, 2, 3 and 4, wherein the sum of p and z is 2, 3 or 4;
s is independently an integer selected from 1 and 2;
[ligand] in each case independently denotes a monosaccharide; and
[tether], [linker] and [cargo] are as defined herein,

with the proviso that when p is 0, at least one [tether]-[linker] moiety comprises a phosphoryl guanidine or N-sulfonylphosphoramidate group (e.g., a group as defined herein).

It will be appreciated that the groups attached to the ‘splitter’ may be directly bonded, or bound via a linking group. It will further be appreciated that any group Z may exist in a deprotonated form, e.g., as O⁻ or S⁻ with a suitable counterion.

In embodiments, p is an integer selected from 1, 2, 3 and 4, and z is an integer selected from 0, 1, 2 and 3. In embodiments, the sum of p and z is 2 or 3. In embodiments, the sum of p and z is 2. In embodiments, the sum of p and z is 3. In embodiments, the sum of p and z is 4. In embodiments, the sum of p and z is 2 and s is 1. In other embodiments, the sum of p and z is 2 and s is 2. In embodiments, the sum of p and z is 3 or 4 and s is 1. In embodiments, the sum of p and z is 3 and s is 1. In embodiments, p is 2, z is 1 and s is 1, and Z is S. In other embodiments, p is 1, z is 2 and s is 1. In embodiments, p is 1, z is 2 and s is 1, and each Z is S. In other embodiments, p is 1, z is 2 and s is 1, and one Z is S, and the other Z is O.

In embodiments, p is 0, and z is an integer selected from 2, 3 and 4. In these embodiments, at least one [tether]-[linker] moiety comprises a phosphoryl guanidine or N-sulfonylphosphoramidate group. In embodiments, z is 2 or 3. In embodiments, z is 2. In embodiments, z is 3. In embodiments, z is 2 and s is 1. In other embodiments, z is 2 and s is 2. In embodiments, z is 3 and s is 1.

In embodiments, the ligand is GalNAc and the spacer is as defined for, e.g., Formula (II) above. Viewed from this aspect the disclosure provides a compound, or a pharmaceutically acceptable salt thereof, having a structure as illustrated by Formula (V):

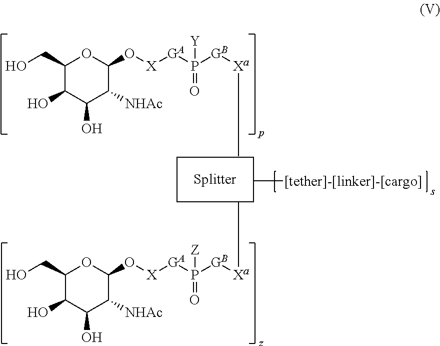

wherein X, Y, Z, X^{a}, G^{A}, G^{B}, p, s, z, Splitter, [tether], [linker] and [cargo] are as defined herein, and wherein the sum of p and z is 2, 3 or 4, with the proviso that when p is 0, at least one [tether]-[linker] moiety comprises a phosphoryl guanidine or N-sulfonylphosphoramidate group (e.g., a group as defined herein).

In embodiments, X in each case is independently optionally substituted —(CH₂)ₘ— or *—[(CH₂)₂O]ₒ(CH₂)₂— (wherein * denotes the point of attachment of the group to G^{A}), wherein each m is independently an integer selected from 3, 4, 5 and 6 (e.g., 3, 4 or 5), and each o is independently an integer selected from 1, 2 and 3 (e.g., 1 or 2). In embodiments, X in each case is independently optionally substituted —(CH₂)₄—.

In embodiments, X^{a} in each case is independently optionally substituted —(CH₂)ₜ— or —C(O)(CH₂)ᵤ—* (wherein * denotes the point of attachment to G^{B}), wherein t and u in each case are integers independently selected from 1, 2, 3, 4, 5 and 6 (e.g., 2, 3, 4 or 5). In embodiments, X^{a} in each case is independently optionally substituted —(CH₂)₃—.

In embodiments, Y in each case is independently selected from an —NH-alkylsulfonyl group (e.g., wherein alkyl denotes optionally substituted —(C₁-C₆)alkyl), an —NH-arylsulfonyl group (e.g., wherein aryl denotes optionally substituted phenyl), and a 1,3-dimethyl-2-(imino)imidazolidine group. In embodiments, Y in each case is independently selected from —NH-mesyl, —NH-phenylsulfonyl, —NH-tosyl and a 1,3-dimethyl-2-(imino)imidazolidine group which is bonded to phosphorus via the imino nitrogen atom.

In embodiments, p is an integer selected from 1, 2, 3 and 4 (e.g., 1, 2 and 3), and z is an integer selected from 0, 1, 2 and 3 (e.g., 0, 1 or 2). In embodiments, the sum of p and z is 2, 3 or 4. In embodiments, the sum of p and z is 2 or 3. In embodiments, the sum of p and z is 2. In embodiments, the sum of p and z is 3. In embodiments, the sum of p and z is 4. In embodiments, the sum of p and z is 2 and s is 1. In other embodiments, the sum of p and z is 2 and s is 2. In embodiments, the sum of p and z is 3 or 4 and s is 1. In embodiments, the sum of p and z is 3 and s is 1. In embodiments, p is 2, z is 1 and s is 1, and Z is S. In other embodiments, p is 1, z is 2 and s is 1. In embodiments, p is 1, z is 2 and s is 1, and each Z is S. In other embodiments, p is 1, z is 2 and s is 1, and one Z is S, and the other Z is O.

In embodiments, p is 0, and z is an integer selected from 2, 3 and 4. In these embodiments, at least one [tether]-[linker] moiety comprises a phosphoryl guanidine or N-sulfonylphosphoramidate group. In embodiments, z is 2 or 3. In embodiments, z is 2. In embodiments, z is 3. In embodiments, z is 2 and s is 1. In other embodiments, z is 2 and s is 2. In embodiments, z is 3 and s is 1.

In embodiments:
X in each case is independently optionally substituted —(CH₂)ₘ— or *—[(CH₂)₂O]ₒ(CH₂)₂— (wherein * denotes the point of attachment of the group to G^{A}), wherein
   each m is independently an integer selected from 3, 4, 5 and 6, and
   each o is independently an integer selected from 1, 2 and 3;
X^{a} in each case is independently optionally substituted —(CH₂)ₜ— or —C(O)(CH₂)ᵤ—* (wherein * denotes the point of attachment to G^{B}), wherein
   t and u in each case are integers independently selected from 1, 2, 3, 4, 5 and 6 (e.g., 2, 3, 4 or 5);
G^{A} and G^{B} in each case are independently selected from O and NH, provided that at least one of the G^{A} and G^{B} attached to the same P atom is O in each case;
Y in each case is independently selected from

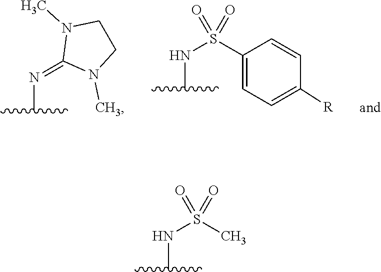

wherein R is independently —CH₃ or —H;
Z in each case is independently selected from —SH and —OH;
p is an integer selected from 1, 2 and 3;
z is an integer selected from 0, 1 and 2; and
s is an integer selected from 1 and 2,

wherein the sum of p and z is 2, 3 or 4.

In embodiments: p is 3; z is 0; s is 1; X in each case is independently optionally substituted —(CH₂)₄—; X^{a} in each case is independently optionally substituted —(CH₂)₃—; G^{A} and G^{B} are both O; and Y in each case is independently selected from

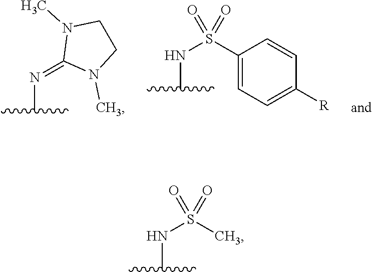

herein R is independently —CH₃ or —H.

Where the compound comprises one or more phosphorothioate groups, each of these may exist in an Rp form, or an Sp form, or in a mixture of Rp and Sp forms. In embodiments, each phosphorothioate group is predominantly (e.g., exclusively) in the Rp form. In other embodiments, each phosphorothioate group is predominantly (e.g., exclusively) in the Sp form. In other embodiments, each phosphorothioate group is in a mixture of Rp and Sp forms, e.g., about half in the Rp form and about half in the Sp form.

In embodiments, the compounds of the disclosure use a splitter as exemplified herein. In embodiments, the splitter is (or comprises) the group

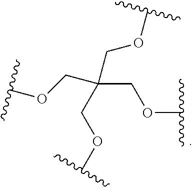 [SMILES: *OCC(CO*)(CO*)CO*]

Viewed from this aspect the disclosure provides a compound, or a pharmaceutically acceptable salt thereof, wherein the compound is of Formula (VI):

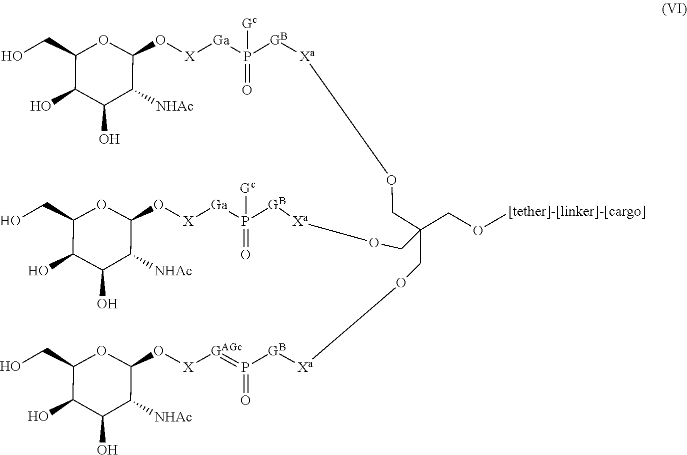

wherein X, G^{A}, G^{B}, X^{a}, [tether], [linker] and [cargo] are as defined herein, and G^{C} in each case is independently selected from Y and Z as defined herein.

In embodiments:
X in each case is independently as defined herein (e.g., independently optionally substituted —(CH₂)ₘ—, wherein m is independently an integer selected from 3, 4, 5 and 6);
G^{A} and G^{B} in each case are independently selected from O and NH, provided that at least one of the G^{A} and G^{B} attached to the same P atom is O in each case;
G^{C} in each case is independently selected from Y and Z, wherein
   Y in each case is independently as defined herein (e.g., independently selected from

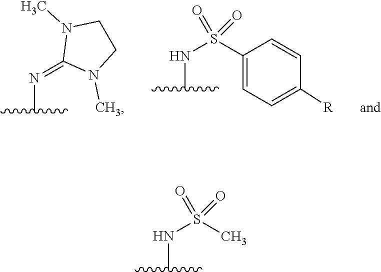

wherein R is independently —CH₃ or —H),
Z in each case is independently selected from —SH and —OH;
X^{a} in each case is independently as defined herein (e.g., independently optionally substituted —(CH₂)ₜ— or —C(O)(CH₂)ᵤ—* (wherein * denotes the point of attachment to G^{B}), and wherein t and u are independently integers selected from 2, 3, 4 or 5); and
[tether], [linker] and [cargo] are as defined herein, with the proviso that: (a) at least one occurrence of G^{C} is Y; and/or (b) the [tether]-[linker] moiety comprises an N-sulfonylphosphoramidate or phosphoryl guanidine group.

In embodiments, X in each case is independently —(CH₂)ₘ—. In embodiments, each X is the same —(CH₂)ₘ—, e.g., wherein m is 4.

In embodiments, G^{A} and G^{B} in each case are O.

In embodiments, one, two, or three of the G^{C} groups are independently selected from Y. In embodiments, all three of the G^{C} groups are independently selected from Y. In embodiments, all three of the G^{C} groups are independently selected from Y, and the [tether]-[linker] moiety comprises an N-sulfonylphosphoramidate (e.g. N-tosyl or N-mesyl phosphoramidate) group, or a phosphoryl guanidine group. In other embodiments, all three of the G^{C} groups are independently selected from Y, and the [tether]-[linker] moiety does not comprise an N-sulfonylphosphoramidate or phosphoryl guanidine group. In embodiments, all three of the G^{C} groups are the same. In embodiments, all three of the G^{C} groups are

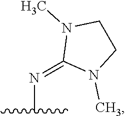 [SMILES: *N=C1N(C)CCN1C]

In other embodiments, all three of the G^{C} groups are

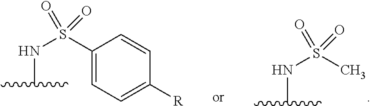

In embodiments, all three of the G^{C} groups are

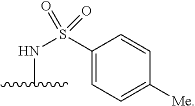 [SMILES: *NS(=O)(=O)c1ccc(C)cc1]

In embodiments, all three of the G^{C} groups are independently selected from Z, and the [tether]-[linker] moiety comprises an N-sulfonylphosphoramidate or phosphoryl guanidine group. In embodiments, the [tether]-[linker] moiety comprises an N-sulfonylphosphoramidate group is selected from:

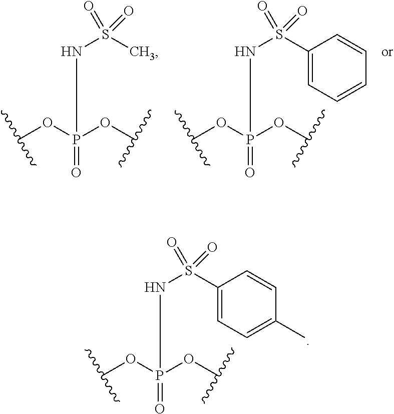 [SMILES: *OP(=O)(NS(C)(=O)=O)O* ; *OP(=O)(NS(=O)(=O)c1ccccc1)O* ; *OP(=O)(NS(=O)(=O)c1ccc(C)cc1)O*]

In other embodiments, the [tether]-[linker] moiety comprises a phosphoryl guanidine group which is:

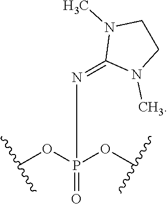 [SMILES: *OP(=O)(N=C1N(C)CCN1C)O*]

In embodiments, X^{a} in each case is independently —(CH₂)ₜ—, wherein t is independently 2, 3 or 4. In embodiments, each X^{a} is the same —(CH₂)ₜ—, e.g. wherein t is independently 3.

In embodiments: X in each case is independently optionally substituted —(CH₂)₄—; X^{a} in each case is independently optionally substituted —(CH₂)₃—; G^{A} and G^{B} are both O; G^{C} in each case is Y; and Y in each case is independently selected from

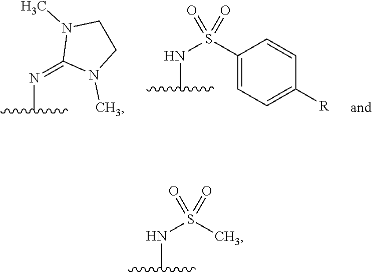

wherein R is independently —CH₃ or —H.

In embodiments, the [tether]-[linker]-[cargo] moiety is selected from:

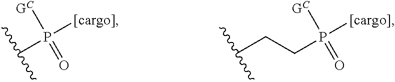

wherein n is independently an integer between 1 and 12 inclusive (e.g. between 1 and 10 inclusive, or between 1 and 6 inclusive), and G^{C} is selected from Y and Z as defined herein. In embodiments, n is independently selected from 3 and 4. In embodiments, n is 3. In other embodiments, n is 4. In other embodiments, n is 5, In other embodiments, n is 6. In embodiments, G^{C} is —OH. In other embodiments, G^{C} is —SH. In other embodiments, G^{C} is

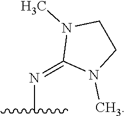 [SMILES: *N=C1N(C)CCN1C]

In embodiments, the [tether]-[linker]-[cargo] moiety is selected from

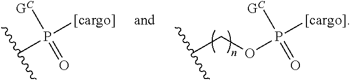

In embodiments, n is 3. In embodiments, n is 4. In embodiments, the [tether]-[linker]-[cargo] moiety is

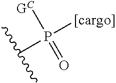

wherein G^{C} is Z (e.g., —OH). In embodiments, the [tether]-[linker]-[cargo] moiety is

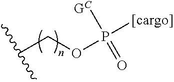

wherein n is selected from 3 and 4, and G^{C} is Z (e.g., —OH) or Y

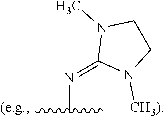

In embodiments, the [tether]-[linker]-[cargo] moiety is

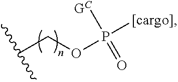

wherein G^{C} is Z (e.g., —OH) or Y

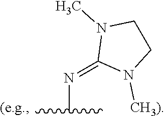

In embodiments, G^{C} is —OH.

In other embodiments, the [tether]-[linker]-[cargo] moiety is

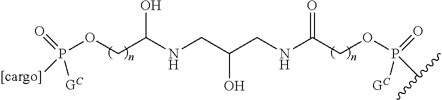

wherein n is independently an integer selected from 1, 2, 3, 4, 5 and 6 (e.g., 4). In embodiments, G^{C} is Z (e.g., —OH).

In other embodiments, the [tether]-[linker]-[cargo] moiety is

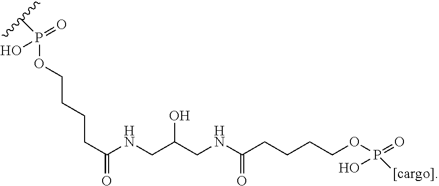

It will be appreciated that the [cargo] will typically be bonded to the [tether]-[linker] moiety via a heteroatom, e.g. an O atom.

In embodiments, the [tether]-[linker]-[cargo] moiety comprises an alkylene group and a phosphate (or modified phosphate) moiety. Viewed from this aspect, the disclosure provides a compound, or a pharmaceutically acceptable salt thereof, wherein the compound is of Formula (VI-A):

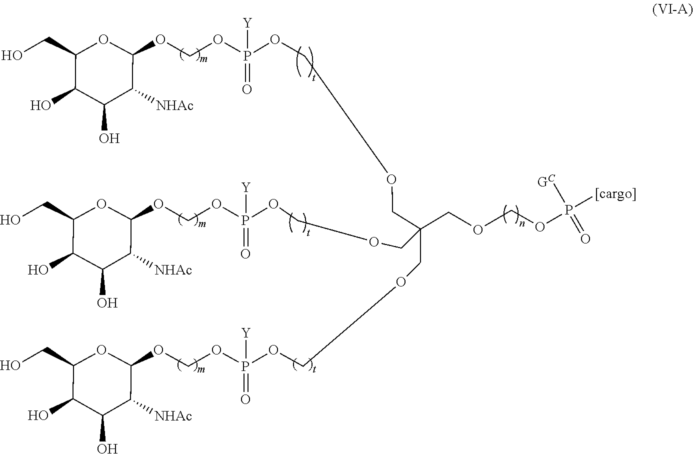

wherein Y, m, t, n and [cargo] are independently as defined herein, and G^{C} is selected from Y and Z as defined herein.

In embodiments:
m in each case is independently an integer selected from 3, 4, 5 and 6;
Y in each case is independently selected from

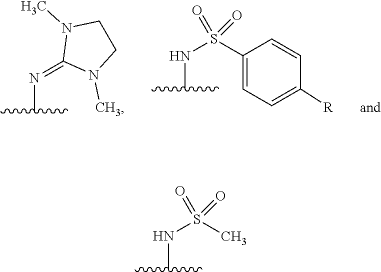

wherein R is independently —CH₃ or —H;
t in each case is independently an integer selected from 2, 3, 4 or 5;
n is 3 or 4; and
G^{C} is selected from Y and Z, wherein
   Y is

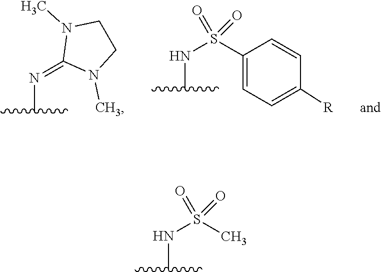

wherein R is independently —CH₃ or —H, and
Z is selected from —SH and —OH.

In embodiments; each m is 4; each t is 3; n is 4; G^{C} is —OH or Y; and Y in each case is independently selected from

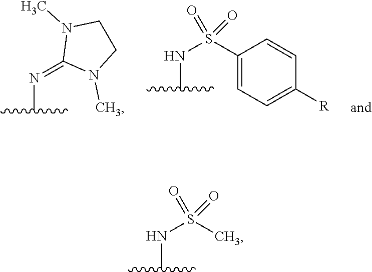

wherein R is independently —CH₃ or —H. In embodiments, each Y is

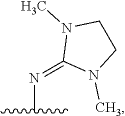 [SMILES: *N=C1N(C)CCN1C]

In embodiments, the splitter is (or comprises) the group

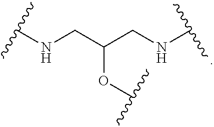 [SMILES: *NCC(CN*)O*]

Viewed from this aspect the disclosure provides a compound, or a pharmaceutically acceptable salt thereof, wherein the compound is of Formula (VII):

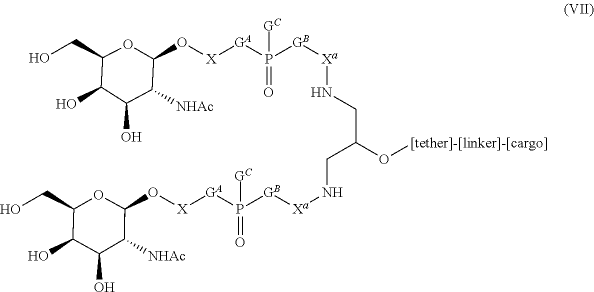

wherein X, G^{A}, G^{B}, X^{a}, [tether], [linker] and [cargo] are as defined herein, and G^{C} in each case is independently selected from Y and Z as defined herein.

In embodiments:
X in each case is independently as defined herein (e.g., independently optionally substituted —(CH₂)ₘ—,
   wherein m is independently an integer selected from 3, 4, 5 and 6);
G^{A} and G^{B} in each case are independently selected from O and NH, provided that at least one of the G^{A} and G^{B} attached to the same P atom is O in each case;
G^{C} in each case is independently selected from Y and Z, wherein
   Y in each case is independently as defined herein (e.g., independently selected from

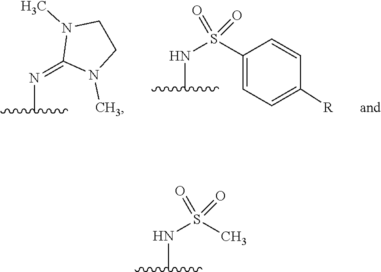

wherein R is independently —CH₃ or —H), and
Z in each case is independently selected from —SH and —OH;
X^{a} in each case is independently as defined herein (e.g., independently optionally substituted —(CH₂)ₜ— or —C(O)(CH₂)ᵤ—* (wherein * denotes the point of attachment to G^{B}), and
   wherein t and u are independently integers selected from 2, 3, 4 or 5); and [tether], [linker] and [cargo] are as defined herein,

with the proviso that: (a) at least one occurrence of G^{C} is Y; and/or (b) the [tether]-[linker] moiety comprises an N-sulfonylphosphoramidate or phosphoryl guanidine group.

In embodiments, both of the G^{C} groups are independently selected from Y. In embodiments, both of the G^{C} groups are the same. In embodiments, both of the G^{C} groups are

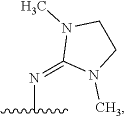 [SMILES: *N=C1N(C)CCN1C]

In embodiments, X^{a} in each case is independently —C(O)(CH₂)ᵤ—* (wherein * denotes the point of attachment to G^{B}), wherein u is independently 3, 4 or 5. In embodiments, each X^{a} is the same —C(O)(CH₂)ᵤ—*, e.g. wherein u is 4.

In embodiments, the [tether]-[linker]-[cargo] moiety is

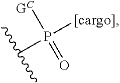

wherein G^{C} is —OH. In embodiments, the splitter is (or comprises) the group

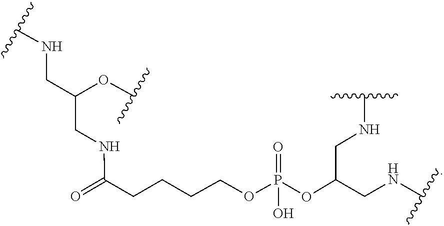 [SMILES: *NCC(CNC(=O)CCCCOP(=O)(O)OC(CN*)CN*)O*]

This is an asymmetric double splitter, which may be formed by linking two splitters from Formula (VII) with a coupling moiety. Viewed from this aspect the disclosure provides a compound, or a pharmaceutically acceptable salt thereof, wherein the compound is of Formula (VIII):

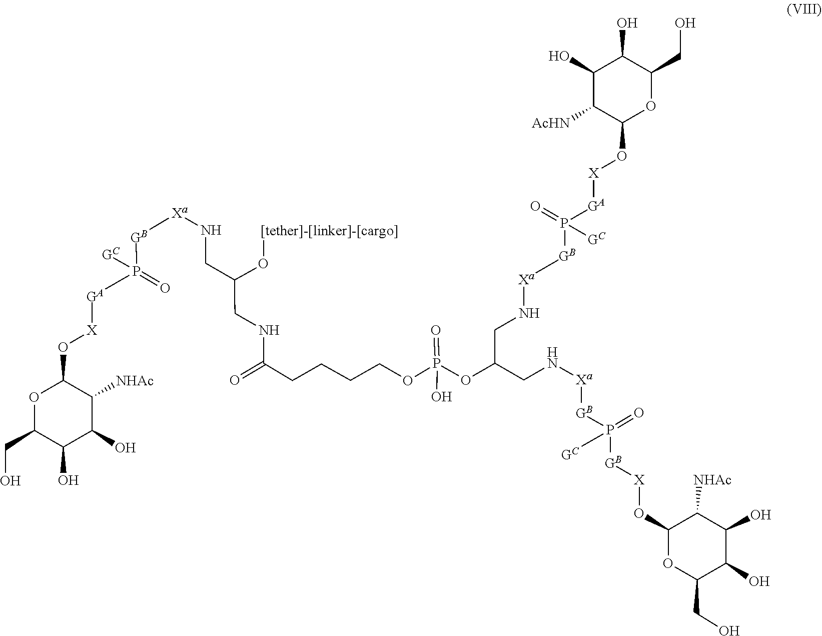

wherein, X, G^{A}, G^{B}, X^{a}, [tether], [linker] and [cargo] are as defined herein, and G^{C} in each case is independently selected from Y and Z as defined herein.

In embodiments:
X in each case is independently as defined herein (e.g., independently optionally substituted —(CH₂)ₘ—,
   wherein m is independently an integer selected from 3, 4, 5 and 6);
G^{A} and G^{B} in each case are independently selected from O and NH, provided that at least one of the G^{A} and G^{B} attached to the same P atom is O in each case;
G^{C} in each case is independently selected from Y and Z, wherein
   Y in each case is independently selected from as defined herein (e.g., independently

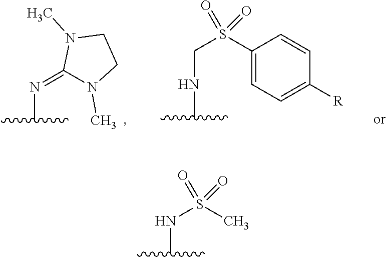

wherein R is independently —CH₃ or —H), and
Z in each case is independently selected from —SH and —OH;
X^{a} in each case is independently as defined herein (e.g., independently optionally substituted —(CH₂)ₜ—, or —C(O)(CH₂)ᵤ—* (wherein * denotes the point of attachment to G^{B}), and
   wherein t and u are independently integers selected from 2, 3, 4 and 5); and
[tether], [linker] and [cargo] are as defined herein,

with the proviso that: (a) at least one occurrence of G^{C} is Y; and/or (b) the [tether]-[linker] moiety comprises an N-sulfonylphosphoramidate or phosphoryl guanidine group.

In embodiments, all three of the G^{C} groups are independently selected from Y. In embodiments, all three of the G^{C} groups are the same. In embodiments, all three of the G^{C} groups are

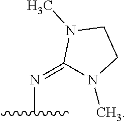 [SMILES: *N=C1N(C)CCN1C]

In embodiments, X^{a} in each case is independently —C(O)(CH₂)ᵤ—* (wherein * denotes the point of attachment to G^{B}), and wherein u is independently an integer selected from 3, 4 or 5. In embodiments, each X^{a} is the same —C(O)(CH₂)ᵤ—*, e.g. wherein u is 4.

In embodiments, the [tether]-[linker]-[cargo] moiety is

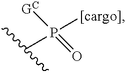

wherein G^{C} is —OH.

In embodiments, the compound is a compound of any one of Examples 1 to 8, 15 and 16, described below. Viewed from this aspect the disclosure provides a compound selected from:

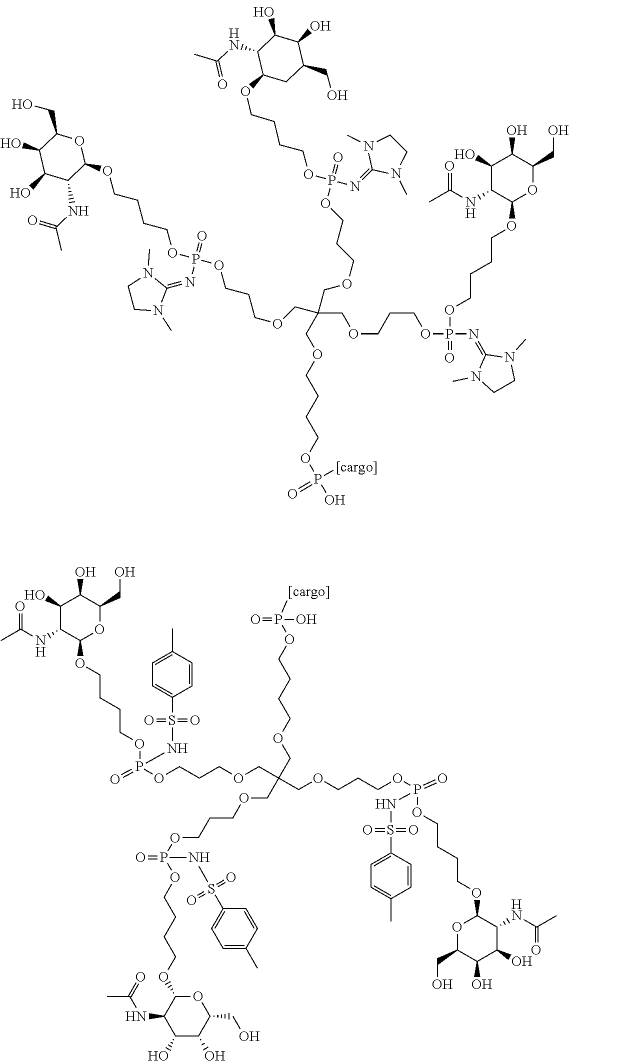

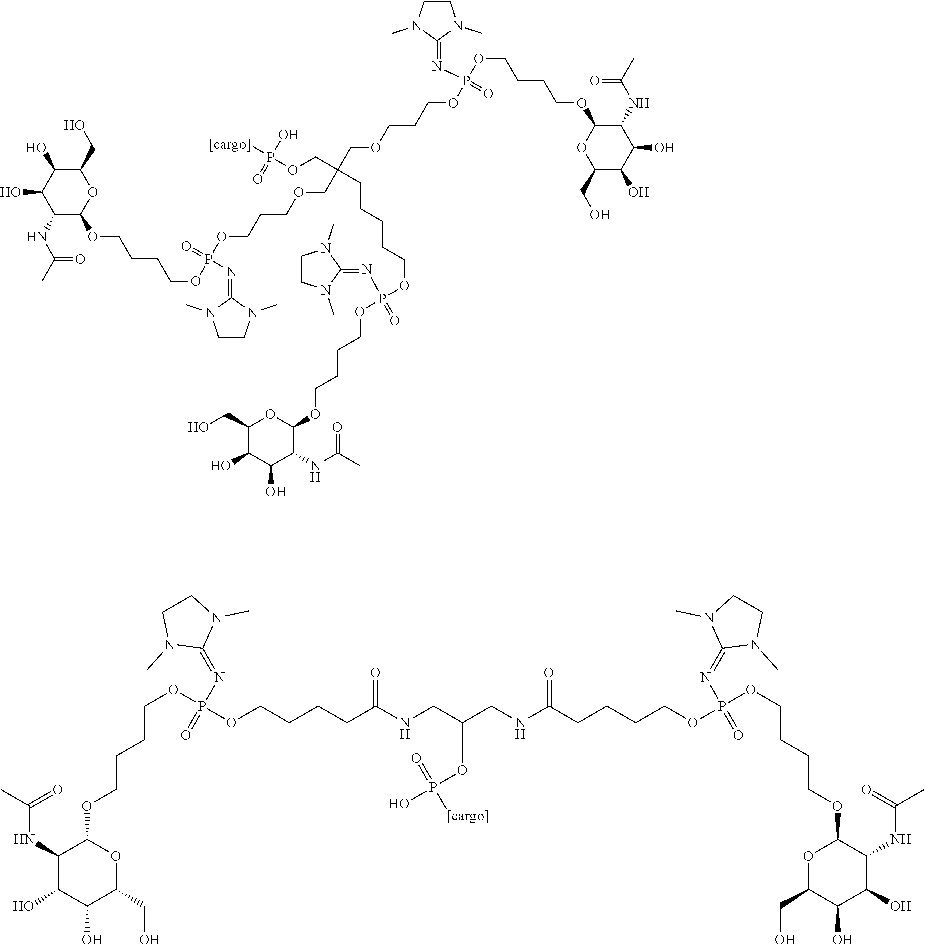

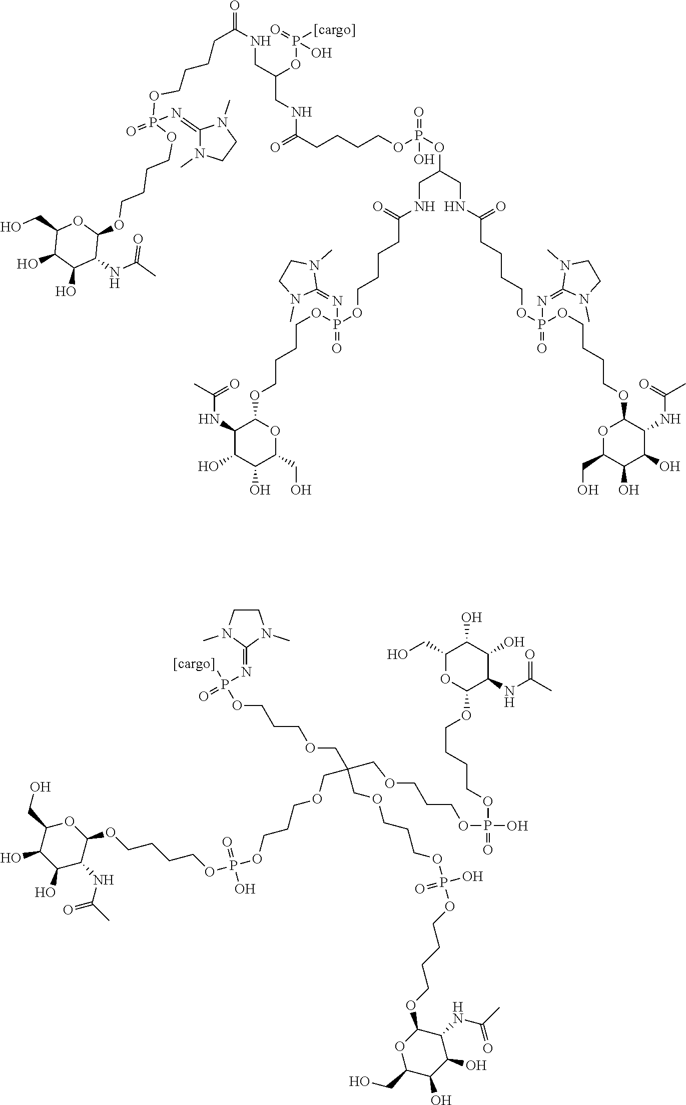

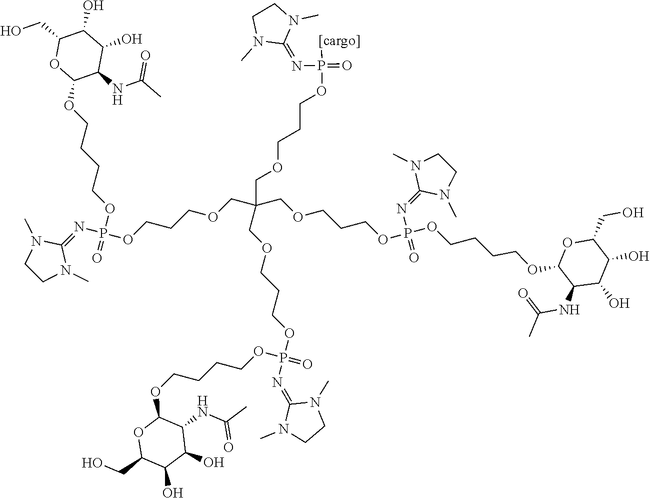

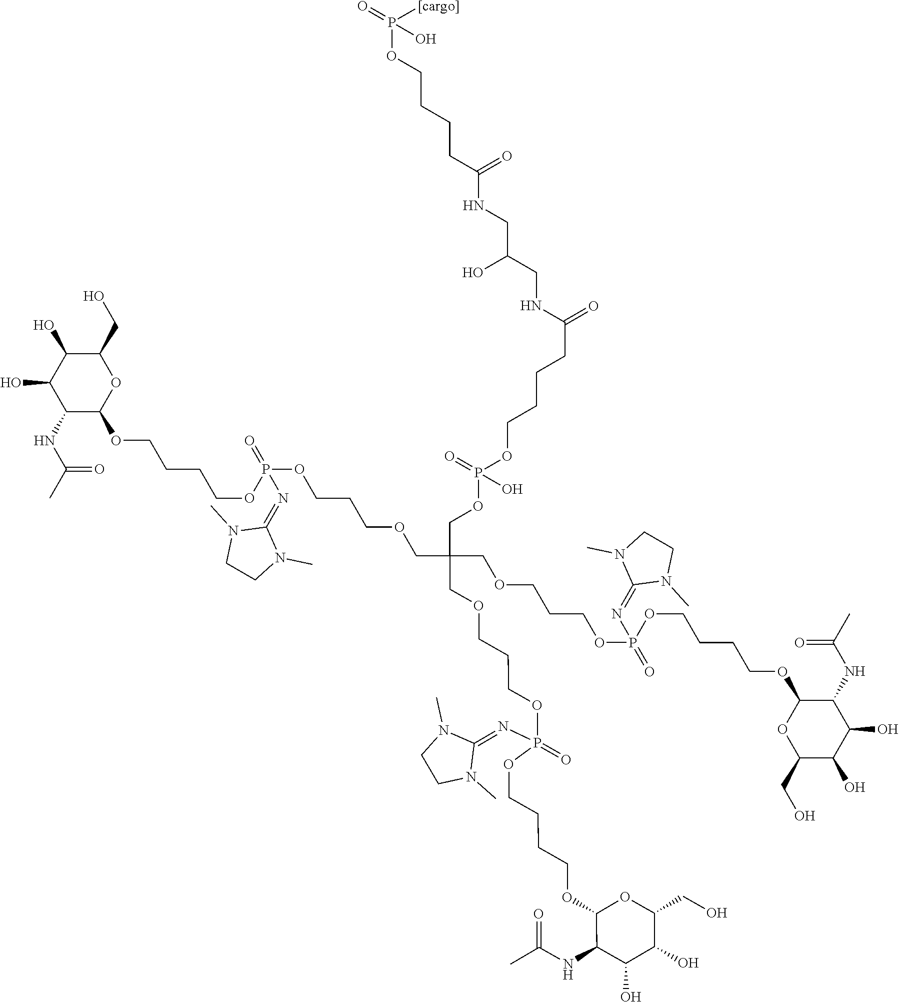

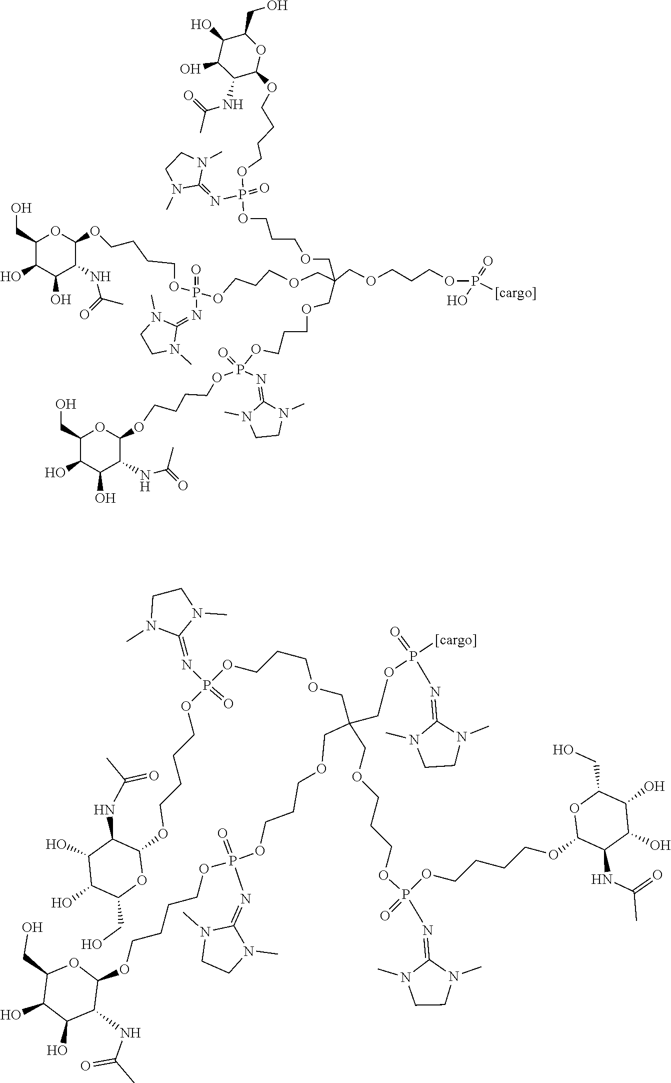

and the pharmaceutically acceptable salts thereof, wherein [cargo] denotes a cargo as defined herein. In embodiments, the oligonucleotide is an ASO (e.g., having a sequence comprising SEQ ID NO: 1) or a siRNA (e.g., having a sequence comprising SEQ ID NO: 2 and/or 3, or having a sequence comprising SEQ ID NO: 4 and/or 5).

In embodiments, the compound is selected from Compounds 3, 4, 5, 12, 13, 14, 15, 16, 17, 18, 19, 20 and 21 as defined hereinafter, and the pharmaceutically acceptable salts thereof.

In embodiments, the compound of the disclosure is characterised as being non-toxic according to a caspase assay (e.g., as measured according to the assay described Example 18 below). In embodiments, the compound is selected from Compounds 14, 15, 16, 17, 18, 20 and 21 as defined hereinafter, and the pharmaceutically acceptable salts thereof.

In embodiments, the compound of the disclosure is characterised according to its activity in knocking down gene expression in human primary hepatocytes (e.g., as measured according to the assay described Example 19 below). In embodiments, the compound has an IC₅₀ value of less than about 2 μM, e.g., an IC₅₀ value of less than about 1.0 μM, 0.5 μM or 0.2 μM. In embodiments, the compound has an IC₅₀ value of less than about 20 nM. In embodiments, the compound is selected from Compounds 4, 12 and 13 as defined hereinafter, and the pharmaceutically acceptable salts thereof. In embodiments, the compound is selected from Compounds 12 and 13, and the pharmaceutically acceptable salts thereof.

In embodiments, the compound of the disclosure is characterised according to its activity in knocking down gene expression in HEK293 cells, e.g. in HEK293 cells overexpressing ASGPR (e.g., as measured according to the assay described Example 20 below). In embodiments, the compound has an IC₅₀ value of less than about 2 μM, e.g., an IC₅₀ value of less than about 1.5 μM, 1.0 μM, 0.5 μM or 0.2 μM. In embodiments, the compound has an IC₅₀ value of less than about 100 nM, e.g. an IC₅₀ value of less than about 75 nM or 50 nM. In embodiments, the compound is selected from Compounds 3, 13, 14, 15, 16, 17, 18, 20 and 21 as defined hereinafter, and the pharmaceutically acceptable salts thereof. In embodiments, the compound is selected from Compounds 3, 13, 16, 18, 20 and 21 as defined hereinafter, and the pharmaceutically acceptable salts thereof. In embodiments, the compound is selected from Compounds 16, 18 and 21 as defined hereinafter, and the pharmaceutically acceptable salts thereof.

In embodiments, the compound of the disclosure is characterised according to its activity in knocking down gene expression in liver tissue in vivo (e.g., as measured according to the assay described Example 21 below). In embodiments, the compound can reduce gene expression in liver tissue after administration by at least 70%, e.g., by at least 75%, 80%, 85% or 90% as compared to control (e.g., as compared to the level before administration, or as compared to the level after administration of a control medium such as PBS). In embodiments, a reduction in gene expression of at least 70% persists for at least 20 days, e.g. for at least 24, 26, 28 or 30 days. In embodiments, the compound is Compound 3 as defined hereinafter or a pharmaceutically acceptable salt thereof. In other embodiments, the compound is Compound 4 as defined hereinafter or a pharmaceutically acceptable salt thereof.

### Nucleic Acids

As defined herein, the compounds of the disclosure are particularly suitable for delivering nucleic acids to cells and tissues. The “cargo” groups of the compounds disclosed herein are thus nucleic acids. The term “nucleic acid” as used herein includes nucleic acids selected from the group consisting of DNA, RNA, PNA and LNA. The nucleic acid may be a functional nucleic acid, e.g., whereby the functional nucleic acid is selected from the group consisting of mRNA, micro-RNA, shRNA, combinations of RNA and DNA, siRNA, siNA, antisense nucleic acid (e.g., antisense oligonucleotide (ASO)), ribozymes, aptamers and spiegelmers. In embodiments, the nucleic acid is selected from siRNA and ASO. In embodiments, the nucleic acid is siRNA. In other embodiments, the nucleic acid is ASO.

The nucleic acids may be of any length and can have any number of nucleotides such that they are effective for the intended purpose (e.g., RNAi). In embodiments, a siRNA ranges from 15 to 30 nucleotides. The duplex region of a double stranded RNA may range from 15 to 30 nucleotide base pairs using the Watson-crick base pairing. The duplex region may have 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 base pairs. In embodiments, the nucleic acid has 19 to 23 base pairs. For example, the nucleic acid may be 19, 20, 21, 22 or 23 base pairs in length. A double stranded RNAi may be blunt ended at one end or on both ends. A double stranded RNAi may have overhangs of 1 or more nucleotides one or both strands at one or both ends. The overhangs may be 1, 2, 3, 4, 5, 6, 7, 8, 9, or 10 nucleotides in length.

For any of the above aspects, the nucleic acid may be a modified nucleic acid. The modification may be selected from substitutions or insertions with analogues of nucleic acids or bases and chemical modification of the base, sugar or phosphate moieties. For example, the nucleic acid may: a) be blunt ended at both ends; b) have an overhang at one end and a blunt end at the other; or c) have an overhang at both ends. One or more nucleotides on the first and/or second strand may be modified, to form modified nucleotides. One or more of the odd numbered nucleotides of the first strand may be modified. One or more of the even numbered nucleotides of the first strand may be modified by at least a second modification, wherein the at least second modification is different from the modification on the one or more add nucleotides. At least one of the one or more modified even numbered nucleotides may be adjacent to at least one of the one or more modified odd numbered nucleotides. The nucleic acid of the disclosure may have the modified nucleotides of the first strand shifted by at least one nucleotide relative to the unmodified or differently modified nucleotides of the second strand.

A nucleic acid of the disclosure may comprise two strands comprising nucleotides, that is able to interfere with gene expression. Inhibition may be complete or partial and can result in downregulation of gene expression in a targeted manner. The nucleic acid may comprise two separate polynucleotide strands; the first strand, which may also be a guide strand; and a second strand, which may also be a passenger strand. The first strand and the second strand may be part of the same polynucleotide molecule that is self-complementary which ‘folds’ to form a double stranded molecule. The nucleic acid may be an siRNA molecule. The first strand may also be referred to as an antisense strand. The second strand may also be referred to as a sense strand.

The nucleic acid may comprise ribonucleotides, modified ribonucleotides, deoxynucleotides, deoxyribonucleotides, or nucleotide analogues. The nucleic acid may further comprise a double stranded nucleic acid portion or duplex region formed by all or a portion of the first strand (also known in the art as a guide strand) and all or a portion of the second strand (also known in the art as a passenger strand). The duplex region is defined as beginning with the first base pair formed between the first strand and the second strand and ending with the last base pair formed between the first strand and the second strand, inclusive.

Depending on the length of a nucleic acid, a perfect match in terms of base complementarity between the first strand and second strand is not necessarily required. However, the first and second strands must be able to hybridise under physiological conditions. The complementarity between the first strand and second strand in the at least one duplex region may be perfect in that there are no nucleotide mismatches or additional/deleted nucleotides in either strand. Alternatively, the complementarity may not be perfect. The complementarity may be at least 70%, 75%, 80%, 85%, 90% or 95%. The first strand and the second strand may each comprise a region of complementarity which comprises at least 15 contiguous nucleotides differing by no more than 3 nucleotides.

Unmodified polynucleotides, particularly ribonucleotides, may be prone to degradation by cellular nucleases, and, as such, modification and/or modified nucleotides may be included in the nucleic acid of the disclosure. One or more nucleotides on the second and/or first strand of the nucleic acid of the disclosure may be modified. Modifications of the nucleic acid of the present disclosure generally provide a powerful tool in overcoming potential limitations including, but not limited to, in vitro and in vivo stability and bioavailability inherent to native RNA molecules. The nucleic acid according to the disclosure may be modified by chemical modifications. Modified nucleic acid can also minimise the possibility of inducing interferon activity in humans. Modification can further enhance the functional delivery of a nucleic acid to a target cell. The modified nucleic acid of the present disclosure may comprise one or more chemically modified ribonucleotides of either or both of the first strand or the second strand. A ribonucleotide may comprise a chemical modification of the base, sugar or phosphate moieties. The ribonucleic acid may be modified by substitution or insertion with analogues of nucleic acids or bases.

One or more nucleotides of a nucleic acid of the present disclosure may be modified. The nucleic acid may comprise at least one modified nucleotide. The modified nucleotide may be on the first strand. The modified nucleotide may be in the second strand. The modified nucleotide may be in the duplex region. The modified nucleotide may be outside the duplex region, i.e., in a single stranded region. The modified nucleotide may be on the first strand and may be outside the duplex region. The modified nucleotide may be on the second strand and may be outside the duplex region. The 3′-terminal nucleotide of the first strand may be a modified nucleotide. The 3′-terminal nucleotide of the second strand may be a modified nucleotide. The 5′-terminal nucleotide of the first strand may be a modified nucleotide. The 5′-terminal nucleotide of the second strand may be a modified nucleotide.

A nucleic acid of the disclosure may have 1 modified nucleotide or a nucleic acid of the disclosure may have about 2-4 modified nucleotides, or a nucleic acid may have about 4-6 modified nucleotides, about 6-8 modified nucleotides, about 8-10 modified nucleotides, about 10-12 modified nucleotides, about 12-14 modified nucleotides, about 14-16 modified nucleotides about 16-18 modified nucleotides, about 18-20 modified nucleotides, about 20-22 modified nucleotides, about 22-24 modified nucleotides, 24-26 modified nucleotides or about 26-28 modified nucleotides. In each case the nucleic acid comprising said modified nucleotides retains at least 50% of its activity as compared to the same nucleic acid but without said modified nucleotides. The nucleic acid may retain 55%, 60%, 65%, 70%, 75%, 80%, 85%, 90%, 95% or 100% or above of its activity as compared to the same nucleic acid but without said modified nucleotides

The modified nucleotide may be a purine or a pyrimidine. At least half of the purines may be modified. At least half of the pyrimidines may be modified. All of the purines may be modified. All of the pyrimidines may be modified. The modified nucleotides may be selected from the group consisting of a 3′ terminal deoxy thymine (dT) nucleotide, a 2′ O methyl modified nucleotide, a 2′ modified nucleotide, a 2′ deoxy modified nucleotide, a locked nucleotide, an abasic nucleotide, a 2′ amino modified nucleotide, a 2′ alkyl modified nucleotide, a morpholino nucleotide, a phosphoramidate, a non-natural base comprising nucleotide, a nucleotide comprising a 5′-phosphorothioate group, a nucleotide comprising a 5′ phosphate or 5′ phosphate mimic and a terminal nucleotide linked to a cholesteryl derivative or a dodecanoic acid bisdecylamide group.

The nucleic acid may comprise a modified nucleotide, wherein the base is selected from 2-aminoadenosine, 2,6-diaminopurine,inosine, pyridin-4-one, pyridin-2-one, phenyl, pseudouracil, 2, 4, 6-trimethoxy benzene, 3-methyl uracil, dihydrouridine, naphthyl, aminophenyl, 5-alkylcytidine (e.g., 5-methyl cytidine), 5-alkyluridine (e.g., ribothymidine), 5-halouridine (e.g., 5-bromouridine), 6-azapyrimidine, 6-alkylpyrimidine (e.g. 6-methyluridine), propyne, quesosine, 2-thiouridine, 4-thiouridine, wybutosine, wybutoxosine, 4-acetyl cytidine, 5-(carboxyhydroxymethyl)uridine, 5′-carboxymethylaminomethyl-2-thiouridine, 5-carboxymethylaminomethyluridine, beta-D-galactosylqueosine, 1-methyladenosine, 1-methylinosine, 2,2-dimethylguanosine, 3-methylcytidine, 2-methyladenosine, 2-methylguanosine, N⁶-methyladenosine (m⁶A), 7-methylguanosine, 5-methoxyaminomethyl-2-thiouridine, 5-methylaminomethyluridine, 5-methylcarbonylmethyluridine, 5-methyloxyuridine, 5-methyl-2-thiouridine, 2-methylthio-N⁶-isopentenyladenosine, beta-D-mannosylqueosine, uridine-5-oxyacetic acid and 2-thiocytidine.

Nucleic acids discussed herein include unmodified RNA as well as RNA which has been modified, e.g., to improve efficacy, and polymers of nucleoside surrogates. Unmodified RNA refers to a molecule in which the components of the nucleic acid, namely sugars, bases, and phosphate moieties, are the same or essentially the same as that which occur in nature, for example as occur naturally in the human body. Modified nucleotide as used herein refers to a nucleotide in which one or more of the components of the nucleic acid, namely sugars, bases, and phosphate moieties, are different from that which occur in nature. While they are referred to as modified nucleotides they will of course, because of the modification, include molecules which are not nucleotides, for example a polynucleotide molecule in which the ribophosphate backbone is replaced with a non-ribophosphate construct that allows hybridisation between strands, i.e., the modified nucleotides mimic the ribophosphate backbone.

Many of the modifications described herein that occur within a nucleic acid will be repeated within a polynucleotide molecule, such as a modification of a base, or a phosphate moiety, or the a non-linking oxygen of a phosphate moiety. In some cases the modification will occur at all of the possible positions/nucleotides in the polynucleotide but in many cases it will not. A modification may only occur at a 3′ or 5′ terminal position, may only occur in terminal regions, such as at a position on a terminal nucleotide or in the last 2, 3, 4, 5, or 10 nucleotides of a strand. A modification may occur in a double strand region, a single strand region, or in both. A modification may occur only in the double strand region of a nucleic acid of the disclosure or may only occur in a single strand region of a nucleic acid of the disclosure. A phosphorothioate modification at a non-linking oxygen position may only occur at one or both termini, may only occur in a terminal region, e.g., at a position on a terminal nucleotide or in the last 2, 3, 4 or 5 nucleotides of a strand, or may occur in duplex and/or in single strand regions, particularly at termini. The 5′ end or 3′ ends may be phosphorylated.

Stability of a nucleic acid of the disclosure may be increased by including particular bases in overhangs, or to include modified nucleotides, in single strand overhangs, e.g., in a 5′ or 3′ overhang, or in both. Purine nucleotides may be included in overhangs. All or some of the bases in a 3′ or 5′ overhang may be modified. Modifications can include the use of modifications at the 2′ OH group of the ribose sugar, the use of deoxyribonucleotides, instead of ribonucleotides, and modifications in the phosphate group, such as phosphorothioate modifications. Overhangs need not be homologous with the target sequence.

Nucleases can hydrolyse nucleic acid phosphodiester bonds. However, chemical modifications to nucleic acids can confer improved properties, and can render oligoribonucleotides more stable to nucleases. Modified nucleic acids, as used herein, can include one or more of:
(i) alteration, e.g., replacement, of one or both of the non-linking phosphate oxygens and/or of one or more of the linking phosphate oxygens (referred to as linking even if at the 5′ and 3′ terminus of the nucleic acid of the disclosure);
(ii) alteration, e.g., replacement, of a constituent of the ribose sugar, e.g., of the 2′ hydroxyl on the ribose sugar;
(iii) replacement of the phosphate moiety with “dephospho” linkers;
(iv) modification or replacement of a naturally occurring base;
(v) replacement or modification of the ribose-phosphate backbone;
(vi) modification of the 3′ end or 5′ end of the RNA, e.g., removal, modification or replacement of a terminal phosphate group or conjugation of a moiety, e.g., a fluorescently labelled moiety, to either the 3′ or 5′ end of RNA.

The terms replacement, modification, alteration, indicate a difference from a naturally occurring molecule.

Examples of modified phosphate groups include phosphorothioate, phosphoroselenates, borano phosphates, borano phosphate esters, hydrogen phosphonates, phosphoroamidates, alkyl or aryl phosphonates and phosphotriesters. Phosphorodithioates have both non-linking oxygens replaced by sulphur. One, each or both non-linking oxygens in the phosphate group can be independently any one of S, Se, B, C, H, N, or OR (R is alkyl or aryl). The phosphate linker can also be modified by replacement of a linking oxygen with nitrogen (bridged phosphoroamidates), sulphur (bridged phosphorothioates) and carbon (bridged methylenephosphonates). The replacement can occur at a terminal oxygen. Replacement of the non-linking oxygens with nitrogen is possible.

A modified nucleotide can include modification of the sugar groups. The 2′ hydroxyl group (OH) can be modified or replaced with a number of different “oxy” or “deoxy” substituents. Examples of “oxy”-2′ hydroxyl group modifications include alkoxy or aryloxy (OR, e.g., R=H, alkyl, cycloalkyl, aryl, aralkyl, heteroaryl or sugar); polyethyleneglycols (PEG), O(CH₂CH₂O)˜CH₂CH₂OR; “locked” nucleic acids (LNA) in which the 2′ hydroxyl is connected, e.g., by a methylene bridge, to the 4′ carbon of the same ribose sugar; O-AMINE (AMINE=NH₂; alkylamino, dialkylamino, heterocyclyl, arylamino, diaryl amino, heteroaryl amino, or diheteroaryl amino, ethylene diamine, polyamino) and aminoalkoxy, O(CH₂)ₙAMINE, (e.g., AMINE=NH₂; alkylamino, dialkylamino, heterocyclyl, arylamino, diaryl amino, heteroaryl amino, or diheteroaryl amino, ethylene diamine, polyamino). “Deoxy” modifications include hydrogen halo; amino (e.g., NH₂; alkylamino, dialkylamino, heterocyclyl, arylamino, diaryl amino, heteroaryl amino, diheteroaryl amino, or amino acid); NH(CH₂CH₂NH)˜ CH₂CH₂-AMINE (AMINE=NH₂; alkylamino, dialkylamino, heterocyclyl, arylamino, diaryl amino, heteroaryl amino, or diheteroaryl amino), —NHC(O)R (R=alkyl, cycloalkyl, aryl, aralkyl, heteroaryl or sugar), cyano; mercapto; alkyl-thio-alkyl; thioalkoxy; and alkyl, cycloalkyl, aryl, alkenyl and alkynyl, which may be optionally substituted with e.g., an amino functionality. Other substituents of certain embodiments include 2′-methoxyethyl, 2′-OCH3, 2′-O-allyl, 2′-C-allyl, and 2′-fluoro.

The sugar group can also contain one or more carbons that possess the opposite stereochemical configuration than that of the corresponding carbon in ribose. Thus, a modified nucleotides may contain a sugar such as arabinose. Modified nucleotides can also include “abasic” sugars, which lack a nucleobase at C—I′. These abasic sugars can further contain modifications at one or more of the constituent sugar atoms.

The 2′ modifications may be used in combination with one or more phosphate linker modifications (e.g., phosphorothioate). The phosphate group can be replaced by non-phosphorus containing connectors. Examples of moieties which can replace the phosphate group include siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo and methyleneoxymethylimino. In certain embodiments, replacements may include the methylenecarbonylamino and methylenemethylimino groups.

The phosphate linker and ribose sugar may be replaced by nuclease resistant nucleotides. Examples include the mophilino, cyclobutyl, pyrrolidine and peptide nucleic acid (PNA) nucleoside surrogates. In certain embodiments, PNA surrogates may be used.

The 3′ and 5′ ends of an oligonucleotide can be modified. Such modifications can be at the 3′ end or the 5′ end or both ends of the molecule. They can include modification or replacement of an entire terminal phosphate or of one or more of the atoms of the phosphate group. For example, the 3′ and 5′ ends of an oligonucleotide can be conjugated to other functional molecular entities such as labelling moieties, e.g., fluorophores (e.g., pyrene, TAMRA, fluorescein, Cy3 or Cy5 dyes) or protecting groups (based, e.g., on sulphur, silicon, boron or an ester). The functional molecular entities can be attached to the sugar through a phosphate group and/or a linker. The terminal atom of the linker can connect to or replace the linking atom of the phosphate group or the C-3′ or C-5′ O, N, S or C group of the sugar. Alternatively, the linker can connect to or replace the terminal atom of a nucleotide surrogate (e.g., PNAs). These spacers or linkers can include, e.g., —(CH₂)ₙ—, —(CH₂)—NH—, —(CH₂)˜O—, —(CH₂)ₙS—, O(CH₂CH₂O)ₙCH₂CH₂OH (e.g., where n=3 or 6), abasic sugars, amide, carboxy, amine, oxyamine, oxyimine, thioether, disulfide, thiourea, sulfonamide, or morpholino, or biotin and fluorescein reagents. The 3′ end can be an —OH group.

Other examples of terminal modifications include dyes, intercalating agents (e.g., acridines), cross-linkers (e.g., psoralene, mitomycin C), porphyrins (TPPC4, texaphyrin, Sapphyrin), polycyclic aromatic hydrocarbons (e.g., phenazine, dihydrophenazine), artificial endonucleases (e.g., EDTA), lipophilic carriers (e.g., cholesterol, cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid, 03-(oleoyl)lithocholic acid, 03-(oleoyl)cholenic acid, dimethoxytrityl, or phenoxazine) and peptide conjugates (e.g., antennapedia peptide, Tat peptide), alkylating agents, phosphate, amino, mercapto, PEG (e.g., PEG-40K), MPEG, [MPEG]2, polyamino, alkyl, substituted alkyl, radiolabelled markers, enzymes, haptens (e.g., biotin), transport/absorption facilitators (e.g., aspirin, vitamin E, folic acid), synthetic ribonucleases (e.g., imidazole, bisimidazole, histamine, imidazole clusters, acridine-imidazole conjugates, Eu3+ complexes of tetraazamacrocycles).

Terminal modifications can be added for a number of reasons, including to modulate activity or to modulate resistance to degradation. Terminal modifications useful for modulating activity include modification of the 5′ end with phosphate or phosphate analogs. Nucleic acids of the disclosure, on the first or second strand, may be 5′ phosphorylated or include a phosphoryl analog at the 5′ prime terminus. 5′-phosphate modifications include those which are compatible with RISC mediated gene silencing. Suitable modifications include: 5′-monophosphate ((HO)₂(O)P—O-5′); 5′-diphosphate ((HO)₂(O)P—O—P(HO)(O)—O-5′); 5′-triphosphate ((HO)₂(O)P—O—(HO)(O)P—O—P(HO)(O)—O-5′); 5′-guanosine cap (7-methylated or non-methylated) (7m-G-O-5′-(HO)(O)P—O—(HO)(O)P—O—P(HO)(O)—O-5′); 5′-adenosine cap (Appp), and any modified or unmodified nucleotide cap structure (N—O-5′-(HO)(O)P—O—(HO)(O)P—O—P(HO)(O)—O-5′); 5′-monothiophosphate (phosphorothioate; (HO)₂(S)P—O-5′); 5′-monodithiophosphate (phosphorodithioate); (HO)(HS)(S)P—O-5′), 5′-phosphorothiolate ((HO)₂(O)P—S-5′); any additional combination of oxygen/sulphur replaced monophosphate, diphosphate and triphosphates (e.g., 5′-alpha-thiotriphosphate, 5′-gamma-thiotriphosphate, etc.), 5′-phosphoramidates ((HO)₂(O)P—NH-5′, (HO)(NH₂)(O)P—O-5′), 5′-alkylphosphonates (R=alkyl=methyl, ethyl, isopropyl, propyl, etc., e.g., RP(OH)(O)—O-5′-, (OH)₂(O)P-5′-CH₂—), 5′-vinylphosphonate, 5′-alkyletherphosphonates (R=alkylether=methoxymethyl (MeOCH₂—), ethoxymethyl, etc., e.g., RP(OH)(O)—O-5′-).

The nucleic acid of the present disclosure may include one or more phosphorothioate modifications on one or more of the terminal ends of the first and/or the second strand. Optionally, each or either end of the first strand may comprise one or two or three phosphorothioate modified nucleotides. Optionally, each or either end of the second strand may comprise one or two or three phosphorothioate modified nucleotides. Optionally, both ends of the first strand and the 5′ end of the second strand may comprise two phosphorothioate modified nucleotides. By phosphorothioate modified nucleotide it is meant that the linkage between the nucleotide and the adjacent nucleotide comprises a phosphorothioate group instead of a standard phosphate group.

Terminal modifications can also be useful for monitoring distribution, and in such cases the groups to be added may include fluorophores, e.g., fluorescein or an Alexa dye. Terminal modifications can also be useful for enhancing uptake, useful modifications for this include cholesterol. Terminal modifications can also be useful for cross-linking an RNA agent to another moiety.

Adenine, guanine, cytosine and uracil are the most common bases found in RNA. These bases can be modified or replaced to provide RNA's having improved properties. For example, nuclease resistant oligoribonucleotides can be prepared with these bases or with synthetic and natural nucleobases (e.g., inosine, thymine, xanthine, hypoxanthine, nubularine, isoguanisine, or tubercidine) and any one of the above modifications. Alternatively, substituted or modified analogs of any of the above bases and “universal bases” can be employed. Examples include 2-aminoadenine, 6-methyl and other alkyl derivatives of adenine and guanine, 2-propyl and other alkyl derivatives of adenine and guanine, 5-halouracil and cytosine, 5-propynyl uracil and cytosine, 6-azo uracil, cytosine and thymine, 5-uracil (pseudouracil), 4-thiouracil, 5-halouracil, 5-(2-aminopropyl)uracil, 5-amino allyl uracil, 8-halo, amino, thiol, thioalkyl, hydroxyl and other 8-substituted adenines and guanines, 5-trifluoromethyl and other 5-substituted uracils and cytosines, 7-methylguanine, 5-substituted pyrrolidines, 6-azapyrimidines and N-2, N-6 and 0-6 substituted purines, including 2-aminopropyladenine, 5-propynyluracil and 5-propynyl cytosine, dihydrouracil, 3-deaza-5-azacytosine, 2-aminopurine, 5-alkyluracil, 7-alkylguanine, 5-alkyl cytosine, 7-deazaadenine, N⁶,N⁶-dimethyladenine, 2,6-diaminopurine, 5-amino-allyl-uracil, N³-methyl uracil, substituted 1,2,4-triazoles, 2-pyridinone, 5-nitroindole, 3-nitropyrrole, 5-methoxyuracil, uracil-5-oxyacetic acid, 5-methoxycarbonylmethyluracil, 5-methyl-2-thiouracil, 5-methoxycarbonylmethyl-2-thiouracil, 5-methylaminomethyl-2-thiouracil, 3-(3-amino-3-carboxypropyl)uracil, 3-methylcytosine, 5-methylcytosine, N<4>-acetyl cytosine, 2-thiocytosine, N⁶-methyladenine, N⁶-isopentyladenine, 2-mcthylthio-N⁶-isopentenyladenine, N-methylguanines, or O-alkylated bases.

Certain moieties may be linked to the 5′ terminus of the first strand or the second strand and includes abasic ribose moiety, abasic deoxyribose moiety, modifications abasic ribose and abasic deoxyribose moieties including 2′ O-alkyl modifications; inverted abasic ribose and abasic deoxyribose moieties and modifications thereof, C6-imino-Pi; a mirror nucleotide including L-DNA and L-RNA; 5′ OMe nucleotide; and nucleotide analogs including 4′,5′-methylene nucleotide; 1-(β-D-erythrofuranosyl)nucleotide; 4′-thio nucleotide, carbocyclic nucleotide; 5′-amino-alkyl phosphate; 1,3-diamino-2-propyl phosphate, 3-aminopropyl phosphate; 6-aminohexyl phosphate; 12-aminododecyl phosphate; hydroxypropyl phosphate; 1,5-anhydrohexitol nucleotide; alpha-nucleotide; threo-pentofuranosyl nucleotide; acyclic 3′,4′-seco nucleotide; 3,4-dihydroxybutyl nucleotide; 3,5-dihydroxypentyl nucleotide, 5′-5′-inverted abasic moiety; 1,4-butanediol phosphate; 5′-amino; and bridging or non-bridging methylphosphonate and 5′-mercapto moieties. The nucleic acids of the disclosure may include one or more inverted nucleotides, for example inverted thymidine or inverted adenine (see, e.g., Takei et al., J Biol Chem (2002) 277(26):23800-23806).

The nucleic acid of the present disclosure may comprise an abasic nucleotide. The nucleic acid may comprise one or more nucleotides on the second and/or first strands that are modified. Alternating nucleotides may be modified, to form modified nucleotides. In alternating nucleotides, one nucleotide may be modified with a first modification, the next contiguous nucleotide may be modified with a second modification and the following contiguous nucleotide is modified with the first modification and so on, where the first and second modifications are different.

### RNA Modifications

Modifications of the siRNA molecules of the present disclosure generally provides a powerful tool in overcoming potential limitations including, but not limited to, in vitro and in vivo stability and bioavailability inherent to native RNA molecules. The siRNA according to the disclosure may be modified by chemical modifications. Modified siRNA can also minimize the possibility of activating interferon activity in humans. Modification can further enhance the functional delivery of a siRNA to a target cell. The modified siRNA of the present disclosure may comprise one or more chemically modified ribonucleotides of either or both of the antisense strand or the sense strand. A ribonucleotide may comprise a chemical modification of the base, sugar or phosphate moieties. The ribonucleic acid may be modified by substitution or insertion with analogues of nucleic acids or bases.

One or more nucleotides of a siRNA of the present disclosure may comprise a modified base. In one aspect, the siRNA comprises at least one nucleotide comprising a modified base. In one embodiment, the modified base in on the antisense strand. In another embodiment, the modified base in on the sense strand. In another embodiment, the modified base is in the duplex region. In another embodiment, the modified base is outside the duplex region, i.e., in a single stranded region. In another embodiment, the modified base is on the antisense strand and is outside the duplex region. In another embodiment, the modified base is on the sense strand and is outside the duplex region. In another embodiment, the 3′-terminal nucleotide of the antisense strand is a nucleotide with a modified base. In another embodiment, the 3′-terminal nucleotide of the sense strand is nucleotide with a modified base. In another embodiment, the 5′-terminal nucleotide of the antisense strand is nucleotide with a modified base. In another embodiment, the 5′-terminal nucleotide of the sense strand is nucleotide with a modified base.

The modified base may be a purine or a pyrimidine. In another embodiment, at least half of the purines are modified. In another embodiment, at least half of the pyrimidines are modified. In another embodiment, all of the purines are modified. In another embodiment, all of the pyrimidines are modified. In another embodiment, the siRNA may comprise a nucleotide comprising a modified base, wherein the base is selected from 2-aminoadenosine, 2,6-diaminopurine, inosine, pyridin-4-one, pyridin-2-one, phenyl, pseudouracil, 2, 4, 6-trimethoxy benzene, 3-methyl uracil, dihydrouridine, naphthyl, aminophenyl, 5-alkylcytidine (e.g., 5-methylcytidine), 5-alkyluridine (e.g., ribothymidine), 5-halouridine (e.g., 5-bromouridine), 6-azapyrimidine, 6-alkylpyrimidine (e.g. 6-methyluridine), propyne, quesosine, 2-thiouridine, 4-thiouridine, wybutosine, wybutoxosine, 4-acetylcytidine, 5-(carboxyhydroxymethyl)uridine, 5′-carboxymethylaminomethyl-2-thiouridine, 5-carboxymethylaminomethyluridine, beta-D-galactosylqueosine, 1-methyladenosine, 1-methylinosine, 2,2-dimethylguanosine, 3-methylcytidine, 2-methyladenosine, 2-methylguanosine, N6-methyladenosine, 7-methylguanosine, 5-methoxyaminomethyl-2-thiouridine, 5-methylaminomethyluridine, 5-methylcarbonylmethyluridine, 5-methyloxyuridine, 5-methyl-2-thiouridine, 2-methylthio-N6-isopentenyladenosine, beta-D-mannosylqueosine, uridine-5-oxyacetic acid and 2-thiocytidine.

In another aspect, a siRNA of the present disclosure comprises an abasic nucleotide. As used herein, a nucleotide with a modified base does not include abasic nucleotides.

### Modifications to Sugar Moiety

Another aspect relates to modifications to a sugar moiety. One or more nucleotides of a siRNA of the present disclosure may comprise a modified ribose moiety. Modifications at the 2′-position where the 2′-OH is substituted include the non-limiting examples selected from alkyl, substituted alkyl, alkaryl-, arylalkyl-, —F, —Cl, —Br, —CN, —CF₃, —OCF₃, —OCN, —O-alkyl, —S-alkyl, HS-alkyl-O, —O-alkenyl, —S-alkenyl, —N-alkenyl, —SO-alkyl, -alkyl-OSH, -alkyl-OH, —O-alkyl-OH, —O-alkyl-SH, —S-alkyl-OH, —S-alkyl-SH, -alkyl-S-alkyl, -alkyl-O-alkyl, —ONO₂, —NO₂, —N₃, —NH₂, alkylamino, dialkylamino-, aminoalkyl-, aminoalkoxy, aminoacid, aminoacyl-, —ONH₂, —O-aminoalkyl, —O-aminoacid, —O-aminoacyl, heterocycloalkyl-, heterocycloalkaryl-, aminoalkylamino-, polyalklylamino-, substituted silyl-, methoxyethyl-(MOE), alkenyl and alkynyl. “Locked” nucleic acids (LNA) in which the 2′ hydroxyl is connected, e.g., by a methylene bridge, to the 4′ carbon of the same ribose sugar is further included as a 2′ modification of the present disclosure. In embodiments, substituents are 2′-methoxyethyl, 2′-O—CH₃, 2′-O-allyl, 2′-C-allyl, and 2′-fluoro (2′-F).

### Modified Groups

In one aspect, the antisense duplex region comprises a plurality of groups of modified nucleotides, referred to herein as “modified groups”, wherein each modified group consists of one or more identically modified nucleotides, wherein each modified group is flanked on one or both sides by a second group of nucleotides, referred to herein as “flanking groups”, wherein each said flanking group consists of one or more nucleotides that are either unmodified or modified in a manner different from the nucleotides of said modified group. In one embodiment, each modified group in the antisense duplex region is identical, i.e., each modified group consists of an equal number of identically modified nucleotides. In another embodiment, each flanking group has an equal number of nucleotides. In another embodiment, each flanking group is identical. In another embodiment, the nucleotides of said modified groups in the antisense duplex region comprise a modified base. In another embodiment, the nucleotides of said modified groups comprise a modified phosphate backbone. In another embodiment, the nucleotides of said modified groups comprise a modified 2′ position.

In another aspect, the sense duplex region comprises a plurality of groups of modified groups, wherein each modified group consists of one or more identically modified nucleotides, wherein each modified group is flanked on one or both sides by a flanking group, wherein each said flanking group consists of one or more nucleotides that are either unmodified or modified in a manner different from the nucleotides of said modified group. In one embodiment, each modified group in the sense duplex region is identical. In another embodiment, each flanking group has an equal number of nucleotides. In another embodiment, each flanking group is identical. In another embodiment, the nucleotides of said modified groups in the sense duplex region comprise a modified base. In another embodiment, the nucleotides of said modified groups comprise a modified phosphate backbone. In another embodiment, the nucleotides of said modified groups comprise a modified 2′ position.

In another aspect, the antisense duplex region and the sense duplex region each comprise a plurality of modified groups, wherein each modified group consists of one or more identically modified nucleotides, wherein each modified group is flanked on one or both sides by a flanking group, wherein each said flanking group consists of one or more nucleotides that are either unmodified or modified in a manner different from the nucleotides of said modified group. In one embodiment, each modified group in the antisense duplex region and the sense duplex region are identical. In another embodiment, each flanking group in the antisense duplex region and the sense duplex region each have an equal number of nucleotides. In another embodiment, each flanking group in the antisense duplex region and in the sense duplex region are identical. In another embodiment, the nucleotides of said modified groups in the antisense duplex region and the sense duplex region each comprise the same modified groups and the same flanking groups. In another embodiment, the nucleotides of said modified groups in the antisense duplex region and the sense duplex region each comprise a modified base. In another embodiment, the nucleotides of said modified groups in the antisense duplex region and the sense duplex region each comprise a modified phosphate backbone. In another embodiment, the nucleotides of said modified groups in the antisense duplex region and the sense duplex region each comprise a modified 2′ position.

### Modifications to the Phosphate Backbone

Another aspect relates to modifications to a phosphate backbone. All or a portion of the nucleotides of the siRNA of the disclosure may be linked through phosphodiester bonds, as found in unmodified nucleic acid. A siRNA of the present disclosure however, may comprise a modified phosphodiester linkage. The phosphodiester linkages of either the antisense stand or the sense strand may be modified to independently include at least one heteroatom selected from nitrogen and sulphur. In one embodiment, a phosphoester group connecting a ribonucleotide to an adjacent ribonucleotide is replaced by a modified group. In one embodiment, the modified group replacing the phosphoester group is selected from phosphorothioate, methylphosphonate, phosphorodithioate or phosphoramidate group.

5′ and 3′ End Modifications The siRNA of the present disclosure may include nucleic acid molecules comprising one or more modified nucleotides, abasic nucleotides, acyclic or deoxyribonucleotide at the terminal 5′- or 3′-end on either or both of the sense or antisense strands. The 5′-end nucleotide of the antisense and/or strand may be phosphorylated. In another embodiment, the 5′-end nucleotide of the antisense strand is phosphorylated and the 5′-end nucleotide of the sense strand has a free hydroxyl group (5′-OH). In another embodiment, the 5′-end nucleotide of the antisense strand is phosphorylated and the 5′-end nucleotide of the sense strand is modified. In another embodiment the 5′-end nucleotide of the antisense strand carries a 5′E vinylphosphonate.

Modifications to the 5′- and 3′-end nucleotides are not limited to the 5′ and 3′ positions on these terminal nucleotides. Examples of modifications to end nucleotides include, but are not limited to, biotin, inverted (deoxy) abasics, amino, fluoro, chloro, bromo, CN, CF, methoxy, imidazole, carboxylate, thioate, C₁-C₁₀ lower alkyl, substituted lower alkyl, alkaryl or arylalkyl, OCF₃, OCN, O—, S—, or N-alkyl; O—, S—, or N-alkenyl; SO—CH₃; SO₂CH₃; ONO₂; NO₂, N₃; heterocycloalkyl; heterocycloalkaryl; aminoalkylamino; polyalkylamino or substituted silyl, as, among others, described, e.g., in PCT patent publication WO 99/54459, or in European patent EP 0 586 520 B1 or EP 0 618 925 B1, each of which is incorporated by reference in its entirety.

In another embodiment, the terminal 3′ nucleotide or two terminal 3′-nucleotides on either or both of the antisense strand or sense strand is a 2′-deoxynucleotide. In another embodiment, the 2′-deoxynucleotide is a 2′-deoxy-pyrimidine. In another embodiment, the 2′-deoxynucleotide is a 2′ deoxy-thymidine.

shRNA (Short Hairpin Loop RNA) and Linked siRNA

Another aspect relates to shRNA and linked siRNA. The antisense strand and the sense strand may be covalently linked to each other. Such linkage may occur between any of the nucleotides forming the antisense strand and sense strand, respectively and can be formed by covalent or non-covalent linkages. Covalent linkage may be formed by linking both strands one or several times and at one or several positions, respectively, by a compound selected from the group comprising methylene blue and bifunctional groups. In embodiments, bifunctional groups are selected from the group comprising bis(2-chloroethyl)amine, N-acetyl-N′-(p-glyoxylbenzoyl)cystamine, 4-thiouracil and psoralene.

Further, the antisense strand and the sense strand may be linked by a loop structure. The loop structure may be comprised of a non-nucleic acid polymer such as polyethylene glycol. The 5′-end of the antisense strand may be linked to the 3′-terminus of the sense strand or the 3′-end of the antisense strand may be linked to the 5′-end of the sense strand. The loop may consist of a nucleic acid, a locked nucleic acid (LNA), a peptide nucleic acid (PNA), or the loop may be formed by polymers. The length of the loop may be sufficient for linking the two strands covalently in a manner that a back folding can occur through a loop structure or similar structure.

The ribonucleic acid constructs may be incorporated into suitable vector systems. In embodiments, the vector comprises a promoter for the expression of RNAi. The promoter may be selected from any known in the art such as, e.g., pol III, U6, H1 or 7SK.

The nucleic acids according to the disclosure may comprise one or more phosphorothioate internucleotide linkages. The phosphorothioate internucleotide linkages may be distributed across the entire nucleotide sequences and may occur in any number at any position. The nucleic acids can comprise between one to ten phosphorothioate internucleotide linkages. The antisense strand may have at least 1 phosphorothioate modification at each end. The antisense strand may have 1-3 phosphorothioate modifications at each end. For example, the antisense strand may have 2 phosphorothioate modifications at each end. The sense strand may have at least 1 phosphorothioate modification at the 3′ end. The sense strand may have 1-3 phosphorothioate modification at the 3′ end. For example, the sense strand may have 2 phosphorothioate modifications at the 3′ end.

siRNA with Overhangs

An overhang at the 3′-end or 5′ end of the sense strand or the antisense strand may be selected from the group consisting of 1, 2, 3, 4 and 5 nucleotides in length. Alternatively, the siRNA molecule may be blunt-ended on both ends and may have a length of 16 to 29 consecutive nucleotides. In one embodiment, the siRNA molecule is blunt-ended on one end and the double stranded or duplex portion of the siRNA molecule has a length selected from 16 to 29 consecutive nucleotides. In one embodiment, the siRNA molecule has overhangs on both ends on either strand and the double stranded or duplex portion of the siRNA molecule has a length of 16 to 29 consecutive nucleotides. The overhang may comprise at least one deoxyribonucleotides and/or a TT dinucleotide.

It will be appreciated by one skilled in the art that the modification, modifications of the sugar moiety, pattern, 5′ and 3′ end modifications, overhangs, formulations, delivery, dosage and routes of delivery as described above may equally be applied to any type of RNAi molecule and is not limited to siRNAs.

The nucleic acid of the present disclosure can be produced using routine methods in the art including chemically synthesis or expressing the nucleic acid either in vitro (e.g., run off transcription) or in vivo. For example, using solid phase chemical synthesis or using an expression vector. In one embodiment, the expression vector can produce the nucleic acid of the disclosure in a target cell. Methods for the synthesis and purification of the nucleic acid molecules described herein are known to persons skilled in the art.

### Pharmaceutical Compositions

In one aspect, the disclosure provides a pharmaceutical composition comprising a compound described herein (e.g., a compound of Formula (III) or a pharmaceutically acceptable salt thereof), and at least one pharmaceutically acceptable excipient or carrier.

In embodiments, the pharmaceutical composition comprises a compound comprising Formula (I) or a pharmaceutically acceptable salt thereof. In embodiments, the pharmaceutical composition comprises a compound comprising Formula (II) or a pharmaceutically acceptable salt thereof. In embodiments, the pharmaceutical composition comprises a compound of Formula (III) or a pharmaceutically acceptable salt thereof. In embodiments, the pharmaceutical composition comprises a compound of Formula (IV) or a pharmaceutically acceptable salt thereof. In embodiments, the pharmaceutical composition comprises a compound of Formula (V) or a pharmaceutically acceptable salt thereof. In embodiments, the pharmaceutical composition comprises a compound of Formula (VI) or a pharmaceutically acceptable salt thereof. In embodiments, the pharmaceutical composition comprises a compound of Formula (VI-A) or a pharmaceutically acceptable salt thereof. In embodiments, the pharmaceutical composition comprises a compound of Formula (VII) or a pharmaceutically acceptable salt thereof. In embodiments, the pharmaceutical composition comprises a compound of Formula (VIII) or a pharmaceutically acceptable salt thereof.

Nucleic acids and conjugated compounds can be delivered to cells, both in vitro and in vivo, by a variety of methods known to those skilled in the art, including direct contact with cells or by combination with one or more agents that facilitate targeting and/or delivery into cells. Such agents and methods include lipoplexes, liposomes, iontophoresis, hydrogels, cyclodextrins, nanocapsules, micro- and nanospheres and proteinaceous vectors. The nucleic acid/vehicle combination may be locally delivered in vivo by direct injection or by use of an infusion pump.

The composition of the disclosure may comprise surface-modified liposomes containing poly(ethylene glycol) lipids (PEG-modified, or long-circulating liposomes or stealth liposomes). These formulations offer a method for increasing stability of a liposome or lipoplex solutions by preventing their aggregation and fusion. The formulations also have the added benefit in vivo of resisting opsonization and elimination by the mononuclear phagocytic system (MPS or RES), thereby enabling longer blood circulation times and enhanced tissue exposure for the encapsulated drug. Such liposomes may accumulate selectively in tumors, presumably by extravasation and capture in the neovascularized target tissues. The long-circulating liposomes enhance the pharmacokinetics and pharmacodynamics of DNA and RNA, particularly compared to conventional cationic liposomes which are known to accumulate in tissues of the MPS (see, e.g., Liu et al, J. Biol. Chem. (1995) 42:24864-24780; and PCT publication Nos. WO 96/10391; WO 96/10390; and WO 96/10392, the contents of each of which are incorporated herein in their entirety). Long-circulating liposomes may also protect siRNA from nuclease degradation.

The pharmaceutical compositions of the present disclosure may be used as medicaments or as diagnostic agents. For example, one or more compounds (e.g., siRNA conjugates) of the disclosure can be combined with a delivery vehicle (e.g., liposomes) and excipients, such as carriers or diluents. Other agents such as preservatives and stabilizers can also be added. Methods for the delivery of nucleic acid-containing molecules are known in the art and within the knowledge of the person skilled in the art. The compounds (e.g., siRNA conjugates) of the present disclosure can also be administered in combination with other therapeutic compounds, either administrated separately or simultaneously, e.g., as a combined unit dose.

The pharmaceutical composition may be a sterile injectable aqueous suspension or solution, or in a lyophilized form. In one embodiment, the pharmaceutical composition comprises lyophilized lipoplexes or an aqueous suspension of lipoplexes. In embodiments, the lipoplexes comprise a compound of the disclosure. Such lipoplexes may be used to deliver the compounds of the disclosure to a target cell either in vitro or in vivo.

The pharmaceutical compositions and medicaments of the present disclosure may be administered to a subject (e.g., a mammal) in a pharmaceutically effective dose. The mammal may be selected from humans, dogs, cats, horses, cattle, pig, goat, sheep, mouse, rat, hamster and guinea pig. In embodiments, the subject is human.

A compound or composition of the disclosure (e.g., a composition that includes a double stranded siRNA) can be delivered to a subject by a variety of routes. Exemplary routes include: subcutaneous, intramuscular, intradermal, intravenous, topical, rectal, anal, vaginal, nasal, pulmonary, and ocular. Administration may be topical (including ophthalmic, vaginal, rectal, intranasal, transdermal), oral or parenteral. Parenteral administration includes intravenous drip, subcutaneous, intraperitoneal or intramuscular injection, or intrathecal or intraventricular administration. In embodiments, a compound of the disclosure is delivered in vivo by means selected from intravenous, subcutaneous, intramuscular or intradermal injection, or by inhalation. In one embodiment, the compound of the disclosure is delivered by intravenous injection or infusion.

The route and/or site of administration may be chosen to enhance targeting. For example, to target muscle cells, intramuscular injection into the muscles of interest would be a logical choice. Lung cells might be targeted by administering the composition in aerosol form. The vascular endothelial cells could be targeted by coating a balloon catheter with the compound or composition and mechanically introducing the nucleic acid.

The pharmaceutical compositions of the disclosure may be formulated for administration in solid or liquid form, e.g., using conventional carriers or excipients. Compositions may be adapted for, e.g., oral administration (e.g., as a solution, suspension, tablet, or capsule), parenteral administration (e.g., as a solution, dispersion, suspension, or emulsion, or as a dry powder for reconstitution), or topical application (e.g., as a cream, ointment, patch, or spray to be applied to the skin) using techniques known in the art.

### Medical Uses

Compounds of the present disclosure act as modulators of nucleic acids in vivo, which gives them utility in the treatment of numerous disorders and conditions.

Viewed from this aspect, the disclosure provides a compound of the disclosure (or a pharmaceutically acceptable salt thereof, or a pharmaceutical composition comprising the compound or salt) for use in therapy. In a related aspect is provided the use of a compound of the disclosure (or a pharmaceutically acceptable salt thereof, or a pharmaceutical composition comprising the compound or salt) as a medicament. In another related aspect is provided a method of treating a subject in need thereof, the method comprising administering an effective amount of a compound of the disclosure (or a pharmaceutically acceptable salt thereof, or a pharmaceutical composition comprising the compound or salt) to the subject. In another related aspect is provided the use of a compound of the disclosure (or a pharmaceutically acceptable salt thereof, or a pharmaceutical composition comprising the compound or salt) in the manufacture of a medicament.

In particular, compounds of the disclosure can target cells and/or tissues which carry an ASGP receptor and are useful in the treatment of such cells and tissues by delivery of therapeutic nucleic acids. View from this aspect, the disclosure provides a compound of the disclosure (or a pharmaceutically acceptable salt thereof, or a pharmaceutical composition comprising the compound or salt) for use in the treatment of a condition in a subject, which condition is treatable by delivery of a therapeutic nucleic acid to cells and/or tissues of the subject which express ASGPR. In a related aspect, the disclosure provides a method for improving the therapeutic activity of a therapeutic nucleic acid in treating a condition in a subject, which condition is treatable by delivery of the therapeutic nucleic acid to cells and/or tissues of the subject which express ASGPR, the method comprising coupling the therapeutic nucleic acid with one or more molecules to form a compound of the present disclosure (or a pharmaceutically acceptable salt thereof, or a pharmaceutical composition comprising the compound or salt). In another related aspect, the disclosure provides a method for improving the treatment of a condition in a subject, which condition is treatable by delivery of a therapeutic nucleic acid to cells and/or tissues of the subject which express ASGPR, wherein the method comprises delivering the therapeutic nucleic acid as part of a compound of the present disclosure (or a pharmaceutically acceptable salt thereof, or a pharmaceutical composition comprising the compound or salt).

Examples of conditions which may be treatable by delivery of a compound of the disclosure (or a pharmaceutically acceptable salt thereof, or a pharmaceutical composition comprising the compound or salt) include liver diseases (e.g., liver cancer, such as hepatocellular carcinoma), genetic diseases, haemophilia and bleeding disorders, liver fibrosis, non-alcoholic steatohepatitis (NASH), non-alcoholic fatty liver disease (NAFLD), viral hepatitis, rare diseases (e.g. acromegaly), metabolic diseases (e.g. hypercholesterolemia, dyslipidaemia, hypertriglyceridemia), cardiovascular diseases, obesity, thalassemia, liver injury (e.g., drug induced liver injury), hemochromatosis, alcoholic liver diseases, alcohol dependence, anaemia, and anaemia of chronic diseases. In embodiments, the condition is selected from NASH, NAFLD, a metabolic disease and a cardiovascular disease. In embodiments, the condition is NASH.

Viewed from another aspect, the disclosure provides a compound of the disclosure (or a pharmaceutically acceptable salt thereof, or a pharmaceutical composition comprising the compound or salt) for use in the treatment of a condition as defined herein. In a related aspect is provided the use of a compound of the disclosure (or a pharmaceutically acceptable salt thereof, or a pharmaceutical composition comprising the compound or salt) in the treatment of a condition as defined herein. In another related aspect is provided a method of treating a condition in a subject in need thereof, the method comprising administering an effective amount of a compound of the disclosure (or a pharmaceutically acceptable salt thereof, or a pharmaceutical composition comprising the compound or salt) to the subject, wherein the condition is as defined herein. In a further related aspect is provided the use of a compound of the disclosure (or a pharmaceutically acceptable salt thereof, or a pharmaceutical composition comprising the compound or salt) in the manufacture of a medicament for the treatment of a condition as defined herein. In embodiments of the above aspects, the condition is selected from NASH, NAFLD, a metabolic disease (e.g., selected from hypercholesterolemia, dyslipidaemia, and hypertriglyceridemia) and a cardiovascular disease. In embodiments, the condition is NASH.

### Delivery of Nucleic Acids to Cells

A further aspect provides a method of delivering a nucleic acid to a cell using a compound according to the present disclosure (or a pharmaceutically acceptable salt thereof), wherein the cell carries (e.g., expresses) a binding partner for a ligand of the compound. The method comprises the step of contacting the cell with the compound. The method may be used in vitro (e.g., for diagnostic or research purposes) or in vivo (e.g., for diagnostic or therapeutic purposes). In embodiments, the method is an in vitro method. Viewed from this aspect, the disclosure provides an in vitro method of delivering a nucleic acid to a cell using a compound as defined herein (or a pharmaceutically acceptable salt thereof), wherein the cell carries (e.g., expresses) a binding partner for a ligand of the compound, the method comprising the step of contacting the cell with the compound or pharmaceutically acceptable salt thereof.

In embodiments, the binding partner is ASGPR (e.g., human ASGPR) and the compound carries at least one monosaccharide ligand (e.g., the compound carries at least one GalNAc moiety). In embodiments, the binding partner is ASGPR and the compound carries at least one GalNAc moiety (e.g., wherein each ligand is GalNAc). In embodiments, the cell is a hepatocyte, e.g. a mammalian hepatocyte such as a human hepatocyte. In embodiments, the cell is a malignant hepatocyte (e.g., a hepatocellular carcinoma cell).

In embodiments, the method comprises contacting a cell which carries (e.g., expresses) ASGPR (e.g., a hepatocyte cell) with a compound which carries at least one GalNAc moiety, e.g. wherein the compound is a nucleic acid delivery agent comprising Formula (II), or wherein the compound is a compound of Formula (V), Formula (VI), Formula (VI-A), Formula (VII) or Formula (VIII), or a pharmaceutically acceptable salt of the foregoing.

Having been generally described herein, the following non-limiting examples are provided to further illustrate this disclosure.

### EXAMPLES

### General Synthetic Schemes

The following scheme, Scheme 1, illustrates an exemplary way of preparing 5′-functionalised compounds in accordance with the present disclosure and examples:

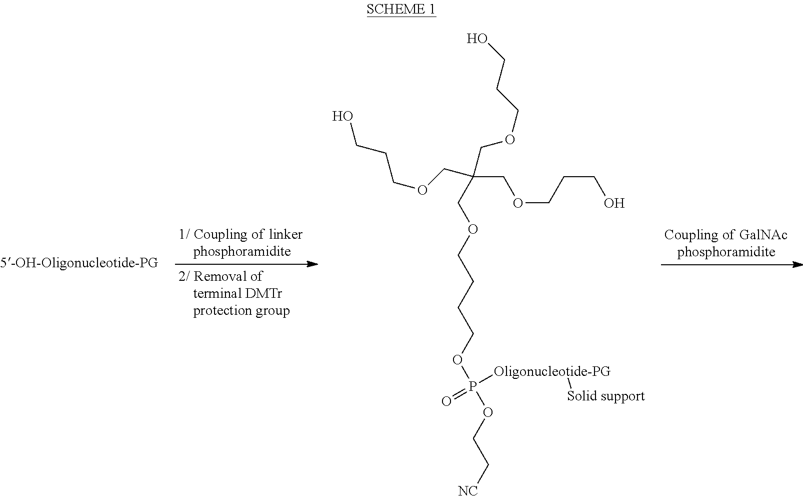

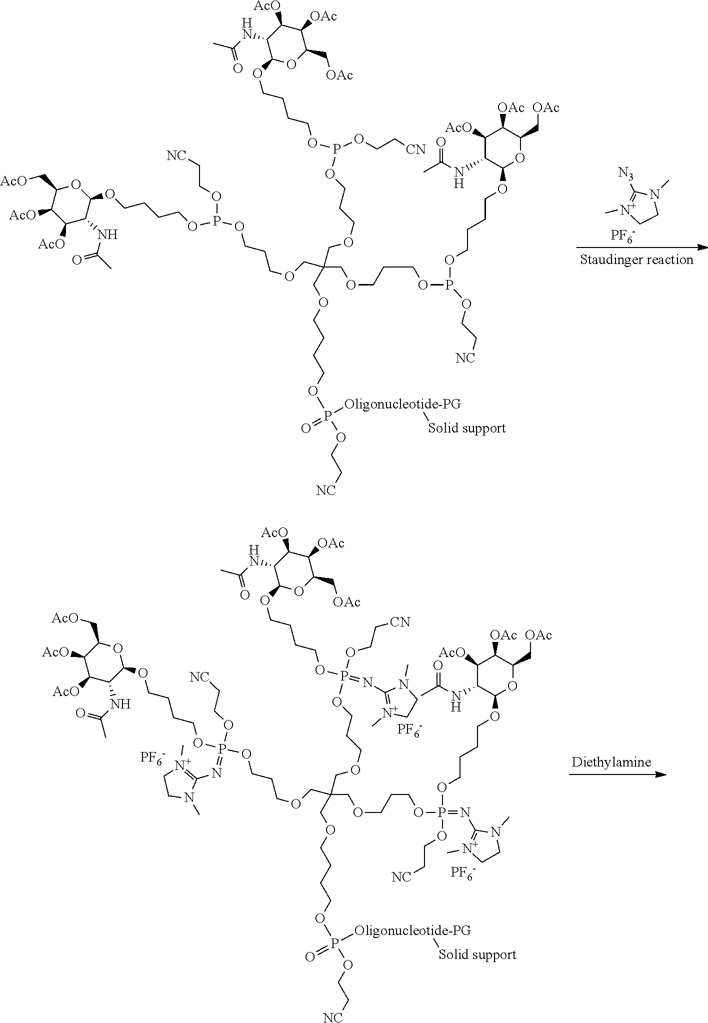

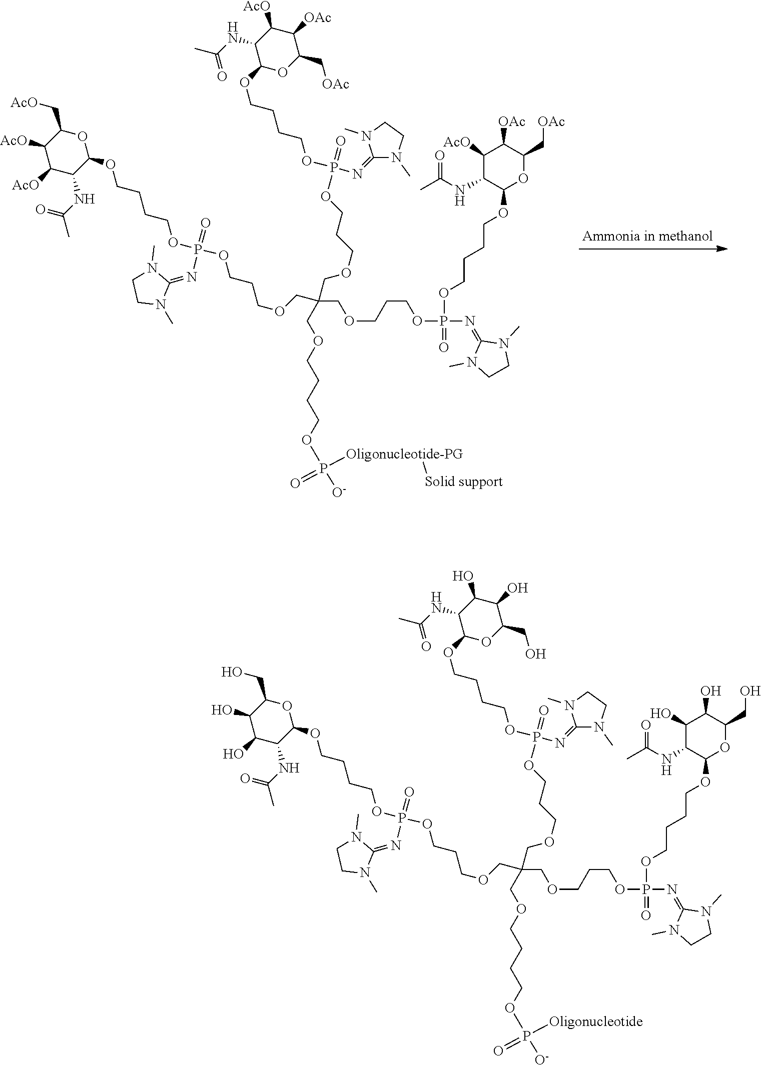

According to Scheme 1 (which illustrates the synthesis of PN-spacer compound in accordance with Example 1 below), a 3′-protected oligonucleotide is coupled with a linker phosphoramidite which contains the [splitter]-[tether]-[linker] moiety having a phosphoramidite at the distal end of the “linker” portion; this reaction may conveniently be performed on solid phase. “PG” denotes a 3′-OH protecting group on the oligonucleotide. The free hydroxyl groups of the “splitter” are then coupled with a GalNAc-containing phosphoramidite to yield a phosphite intermediate which can undergo a Staudinger reaction to form the corresponding iminophosphate (a guanidine phosphate in the present case). The β-cyanoethyl groups are then removed, e.g. using diethylamine, and the compound is released from the solid phase, e.g. using ammonia in methanol.

The following scheme, Scheme 2, illustrates an exemplary way of preparing biantennary GalNAc-containing compounds in accordance with the present disclosure and examples:

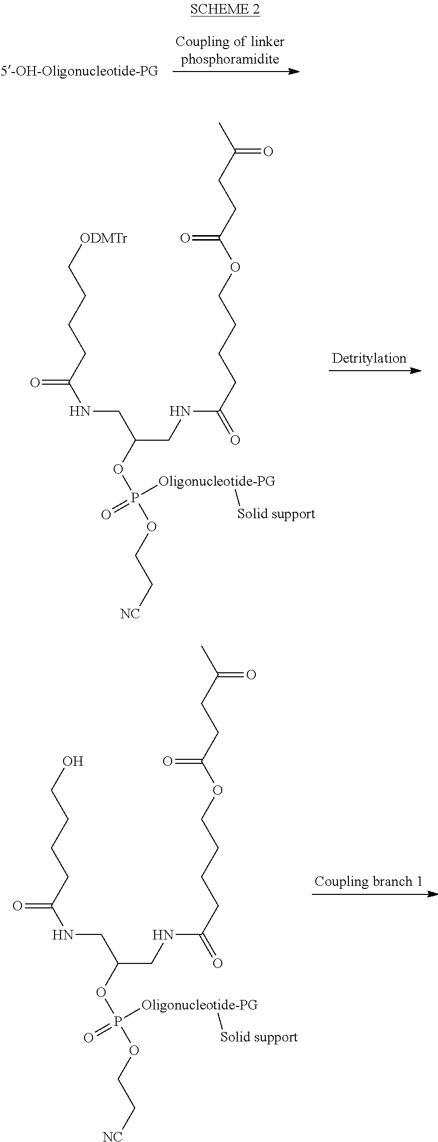

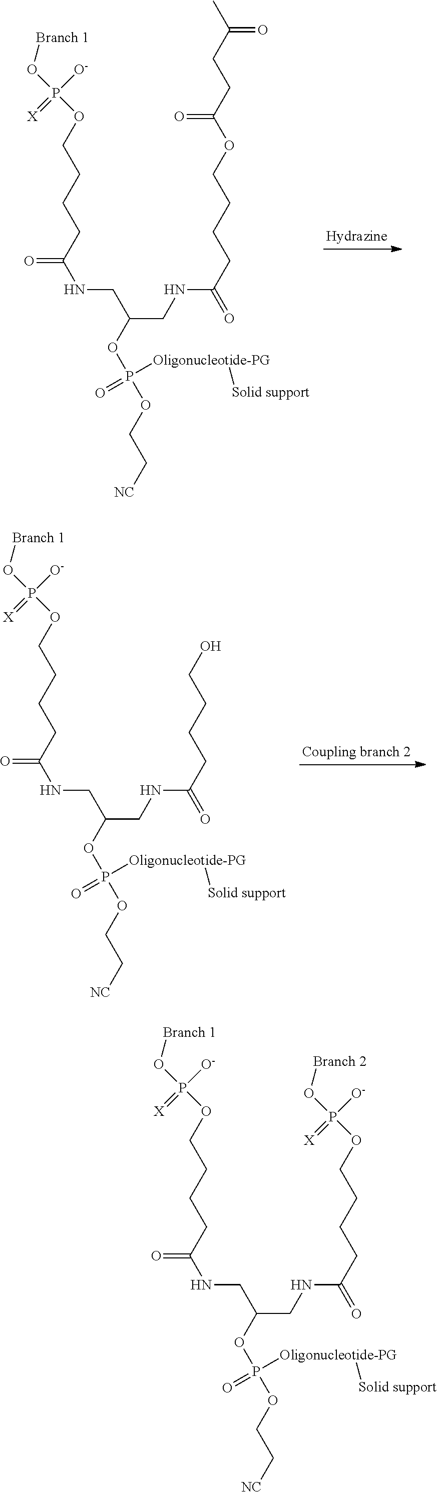

According to Scheme 2, (which illustrates the synthesis of a biantennary compound in accordance with Example 4 below), a 3′-protected oligonucleotide is coupled with a linker phosphoramidite which contains the [splitter]-[tether]-[linker] moiety having a phosphoramidite at the distal end of the “linker” portion; this reaction may conveniently be performed on solid phase. “PG” denotes a 3′-OH protecting group on the oligonucleotide. The “splitter” portion has different protecting groups on its two free arms. One arm is selectively deprotected (shown as a step of DMTr removal) to yield a first free hydroxyl group which is reacted with a first moiety, “Branch 1”, which is e.g. a GalNAc-containing moiety. This reaction may, for example, employ coupling of a GalNAc phosphoramidite followed by the Staudinger reaction to yield a PN-spacer arm (e.g., as shown above in Scheme 1). Alternatively, the reaction may employ coupling of a GalNAc phosphoramidite followed by treatment with water or H₂S (or with iodine or xanthine hydride) to yield a PO- or PS-spacer arm, respectively. The second arm is then selectively deprotected (shown as a step of ester cleavage using hydrazine) to yield a second free hydroxyl group which is reacted with a second moiety, “Branch 2”, which is e.g. a GalNAc-containing moiety. This reaction may, again, yield a PN-spacer arm or a PO— or PS-spacer arm. It will be appreciated that this synthetic scheme may be used to prepare asymmetric biantennary constructs as well as asymmetric triantennary constructs (e.g. as shown in example 5). The compound may then be treated to remove β-cyanoethyl groups and to release it from the solid phase, e.g. as shown in Scheme 1 above.

The following scheme, Scheme 3, illustrates an exemplary way of preparing control compounds in accordance with the present examples:

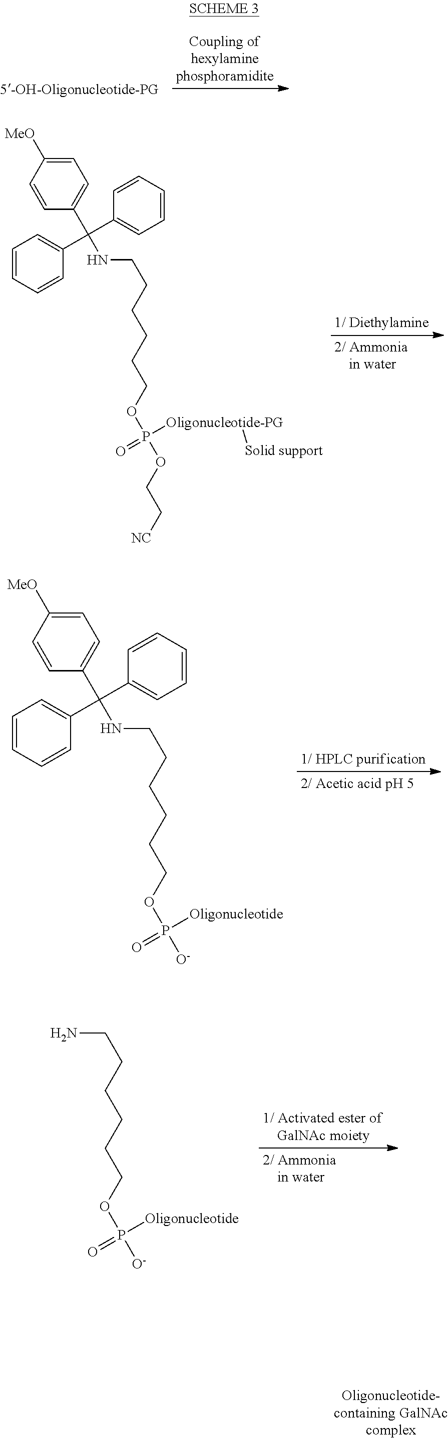

According to Scheme 3, (which illustrates the synthesis of a triantennary compound having amide spacers and linker in accordance with Example 10 below), a 3′-protected oligonucleotide is coupled with a hexyl phosphoramidite which contains the [linker] moiety having a phosphoramidite at its distal end; this reaction may conveniently be performed on solid phase. “PG” denotes a 3′-OH protecting group on the oligonucleotide. The protected amine is then exposed for reaction with the GalNAc-containing carrier; the carrier is provided as an activated ester where the ester is formed on the “tether” portion. The target compound is released from the solid phase, e.g. using ammonia in methanol.

The following scheme, Scheme 4, illustrates an exemplary way of preparing 5′-functionalised compounds in accordance with the present disclosure and examples:

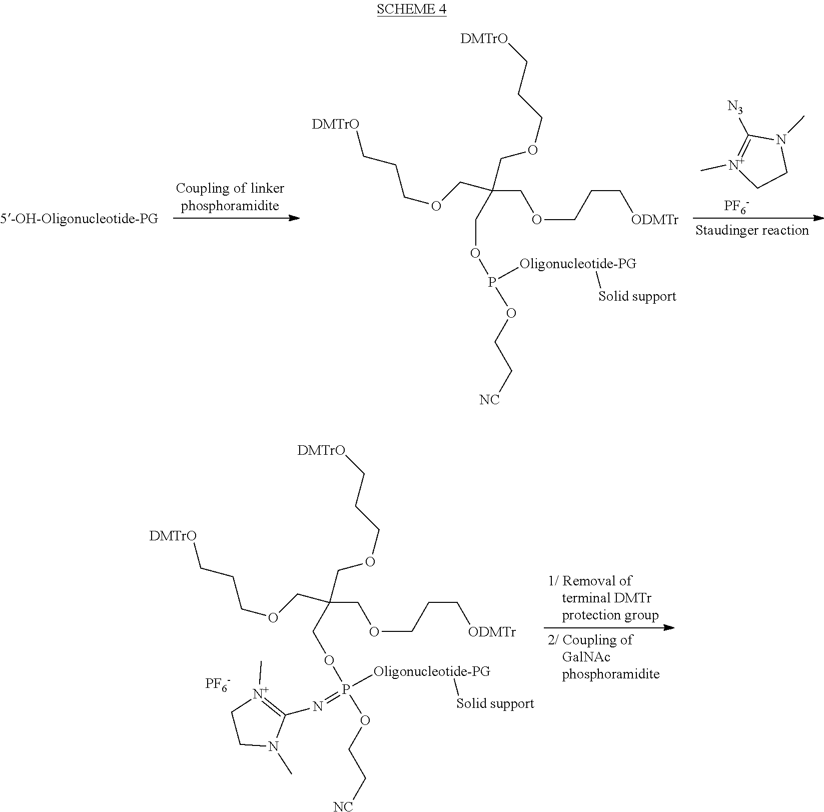

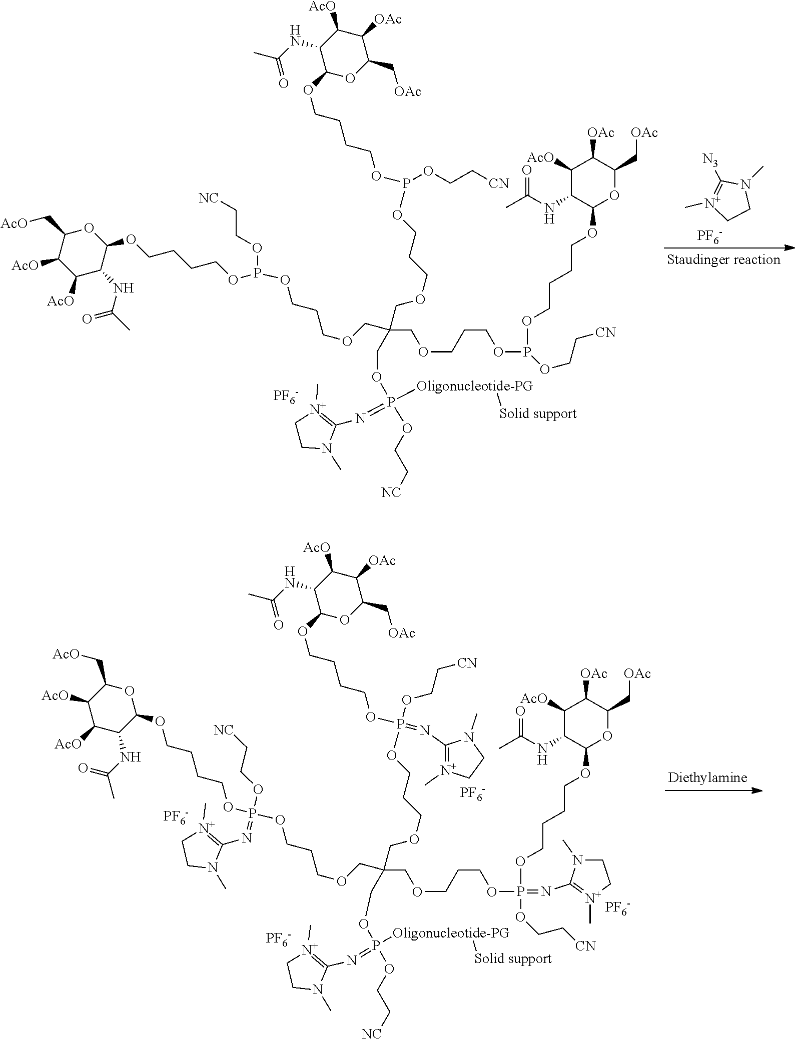

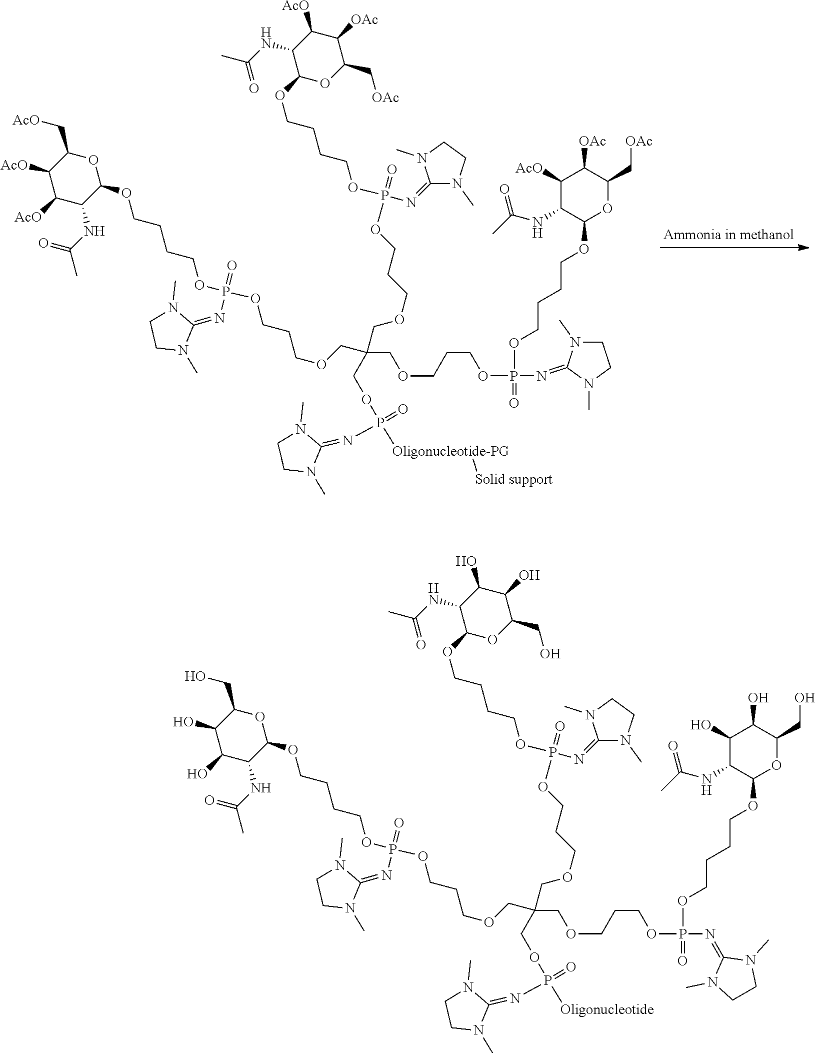

According to Scheme 4 (which illustrates the synthesis of PN-linker and PN-spacer compound in accordance with Example 16 below), a 3′-solid-supported oligonucleotide is coupled with a linker phosphoramidite which contains the [splitter]-[tether]-[linker] moiety having a phosphoramidite at the distal end of the “linker” portion. “PG” denotes protecting groups on the oligonucleotide which may be present, e.g., on the bases and backbone. The intermediate then undergoes a Staudinger reaction to form the corresponding iminophosphate (a guanidine phosphate in the present case). Alternatively, the reaction may employ treatment with iodine or xanthine hydride to yield a PO— or PS-linker, respectively.

Terminal DMTr protecting groups are then removed and the free hydroxyl groups of the “splitter” are then coupled with a GalNAc-containing phosphoramidite to yield a phosphite intermediate which can undergo a further Staudinger reaction to form the corresponding iminophosphate (a guanidine phosphate in the present case). Alternatively, the reaction may employ treatment with iodine or xanthine hydride to yield a PO— or PS-spacer arm, respectively. The β-cyanoethyl groups are then removed, e.g. using diethylamine, and the compound is released from the solid phase, e.g. using ammonia in methanol.

The skilled reader will appreciate that intermediates and reactants used in the above schemes are either commercially available or can be synthesized using known methodology.

### Experimental Techniques

Analytical runs were performed on an ACQUITY PREMIER BEH C18 1.7 m 2.1×100 mm (130 Å) column at 80° C., using a gradient of 35-50% B (100% ACN) in A (10 mM TBAA in 10% ACN/90% H₂O) over 10 min (flow rate 0.5 mL/min). UV purity was measured at 260 nm. MS data were recorded using a Waters Synapt XS spectrometer using electrospray as ionization mode (positive or negative as indicated in report).

All synthetic reactions were performed under an inert atmosphere, unless otherwise stated. In the following examples, when the source of the starting products is not specified, it should be understood that said products are known compounds (e.g., commercially available compounds from suppliers such as Sigma-Aldrich) and/or may be prepared according to known methods, e.g. as described in the literature and patent publications described herein.

### Oligonucleotide Assay Plate Preparation and Analysis (Caspase Assay)

Assay plates containing dose responses of oligonucleotides (ASOs) were prepared in a 384 well plate from 1 mM aqueous stock solutions.

Data were analyzed using Genedata screener. Analyzer uses the following equation to normalize the signal values to the desired signal range:

$$N ( x ) = C R + \frac{x - < cr >}{< sr > - < cr >} ( S R - C R )$$

where: x is the measured raw signal value of a well; <cr> is the median of the measured signal values for the Central Reference (Neutral) wells on a plate; <sr> is the median of the measured signal values for the Scale Reference (Stimulator) wells on a plate; CR is the desired median normalized value for the Central Reference (Neutral); and SR is the desired median normalized value for the Scale Reference (Stimulator).

### Synthesis of Precursors and Cargo Molecules

### Oligonucleotide Synthesis

Oligonucleotides were synthesized on a 10 μmol scale on a K&A system using CUTAG CPG support (Sigma-Aldrich, 25-35 μmol/g). Nucleotide phosphoramidites were purchased from Sigma-Aldrich or WuXi. Linker phosphoramidites were purchased from Glen Research or WuXi. All cEt phosphoramidites were obtained from Pharmaron. The 5′-amino-modifier C6 was obtained from GlenResearch. UV purities were determined using ion-pairing LCMS and are stated at 260 nm. Yields are given based on the initial resin loading and oligonucleotide content of the final product, as calculated from UV absorption.

The sequences of the synthesized oligonucleotides are shown in the table below:

| | |
|---|---|
| ASO | 5′-G*mC*A*T*T*mC*T*A*A*T*A*G*mC*A*G*mC-3′ |
| (MALAT1) | (SEQ ID NO: 1) |
| siRNA | Passenger: 5′-invAb*gcucaacaUAUuugaucagu |
| (ANGPTL3) | a*invAb-3′ (SEQ ID NO: 2) |
| | Guide: 3′-c*GaGuUgUaUaAaCuAgU*c*A*u-5′ |
| | (SEQ ID NO: 3) |
| siRNA | Passenger: 5′-c*A*gCaAaUuCcAuCgU*g*A-3′ |
| (PPIB) | (SEQ ID NO: 4) |
| | Guide: 5′-pu*C*aCgAuGgAaUuUgCuG*u*U-3′ |
| | (SEQ ID NO: 5) |

*denotes phosphorothioate backbone
mC denotes 5′-methylcytidine
CAPS denotes DNA for SEQ ID NO: 1 only
BOLD denotes locked Nucleic Acid
UNDERLINED denotes 2′-fluoro modification for SEQ ID NOs: 2-5
Italics denotes 2′-OMe modification
invAb denotes inverted abasic deoxyribose
p denotes phosphate

### General Synthetic Procedure

This procedure was used unless otherwise indicated.

Phosphoramidites were dissolved to a final concentration to 0.1 M (3 equivalents) in DNA-grade acetonitrile (ACN) prior to use, except for the GalNAc phosphoramidite, dissolved at 0.2 M in DNA-grade ACN. Detritylation was performed using 3 vol-% dichloroacetic acid in DCM (contact time 5×35 s). Di- and tri-antennary linkers were deprotected using double detritylation. Activator 42 was used as activating agent (0.25 M in ACN) for the couplings. Recirculation times of phosphoramidites were 4 min for DNA building blocks, 10 min for all 2′-modified building blocks. Triple 10 min coupling was used for linker phosphoramidites and quadruple coupling for GalNAc phosphoramidite. Xanthane hydride was dissolved in pyridine (0.2 M) and used as thiolation reagent with a contact time of 5 min. Oxidizer solution was purchased from Sigma-Aldrich and used as such with a contact time of 9 s. Tosyl azide (for PTos linkages) or azido-1,3-dimethylimidazolinium hexafluorophosphate (for PGua linkages) were dissolved at 0.2M in ACN and used for Staudinger reaction instead of oxidation/thiolation. The contact time for Staudinger reaction was 6×5 min. Equal volumes of Cap A (9.1 vol % acetic anhydride in tetrahydrofuran (THF)) and Cap B (THF/N-methylimidazole/pyridine 80:10:10 vol-%) were mixed in situ for capping (contact time 50 s). Cyanoethyl backbone removal was performed with 20 vol-% diethylamine in ACN (contact time 7×1 min) after a final 5′-detritylation where required. Oligonucleotides were cleaved from the solid support and further deprotected by treatment with methanolic ammonia (3 M) at 55° C. for 15-20 h and were subsequently purified using ion-pairing HPLC on reverse phase columns. For siRNA compounds, single strands were separately prepared as mQ water solutions of equal concentrations, mixed in equal volumes, warmed to 95 degrees C. for 5 min and then allowed to cool down to room temperature over 1 h.

Special Synthesis Procedure A (e.g. for Use with Scheme 3 Above):

Oligonucleotide was synthesized following general procedure with the exception that 5′-amino-modifier C6 was introduced as last coupling (0.2M, 10 min contact time, double coupling) as last step. The oligonucleotide was purified with the 5′-monomethoxytrityl (MMT) protecting group on. After removal of the MMT group in aqueous acetic acid solution (pH 4.5), the free 5′-NH₂ was reacted with the pentafluorophenyl ester of the GalNAc moiety. Three equivalents of ester were dissolved in ACN and added to a solution of the oligonucleotide in borate buffer (pH 9). Final product was subsequently purified using normal phase HPLC.

### Special Synthesis Procedure B:

Oligonucleotide was synthesized following general procedure but using GalNAc-preloaded Nitto phase support.

### Special Synthesis Procedure C (PN GalNAc on 3′ End and Compounds Containing an Asymmetric Doubler Splitter):

Asymmetric Doubler (Glen Research) was coupled to CUTAG CPG (0.2M, 10 min contact time, double coupling). The DMTr protection group was then removed using standard detritylation conditions. Splitter and GalNAc phosphoramidites were subsequently coupled to the deprotected alcohol (0.2M, 10 min contact time, double coupling) and reacted with azido-1,3-dimethylimidazolinium hexafluorophosphate to obtain guanidine phosphate linkages. The resulting CPG was let sit in 0.5M Hydrazine hydrate in 1:1 pyridine/acetic acid for 15 min to remove the levulinate ester. The support was then rinsed with 5 mL of 1:1 pyridine/acetic acid (3×) and then 5 mL of ACN (3×) before the oligonucleotide was synthesized using standard conditions, starting on the now deprotected alcohol.

### Examples 1 to 17: Synthesis of Test and Control Compounds

The compounds of the Examples have the following general structure:

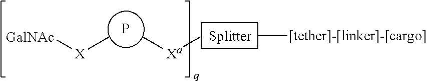

| | Spacer | | | | | | | |
|---|---|---|---|---|---|---|---|---|
| Ex. | X^{a} | P* | X | q | Splitter | Tether* | Linker* | Cargo |
| 1 | ⁿPr | PG | ⁿBu | 3 | C(CH₂O—)₄ | ⁿBu | PO | ASO/ |
| | | | | | | | | siRNA |
| 2 | ⁿPr | PNS | ⁿBu | 3 | C(CH₂O—)₄ | ⁿBu | PO | siRNA |
| 3 | ⁿPr | PG | ⁿBu | 3 | C(CH₂O—)₄ | bond | PO | ASO |
| 4 | pentanoyl | PG | ⁿBu | 2 | —CH(CH₂NH—)2 | bond | PO | ASO |
| 5 | pentanoyl | PG | ⁿBu | 3 | dual^{†} | bond | PO | ASO |
| 6 | ⁿPr | PO | ⁿBu | 3 | C(CH₂O—)₄ | ⁿPr | PG | ASO |
| 7 | ⁿPr | PG | ⁿBu | 3 | C(CH₂O—)₄ | ⁿPr | PG | ASO |
| 8 | ⁿPr | PG | ⁿBu | 3 | C(CH₂O—)₄ | PO | complex^{†} | 3’-ASO |
| 9 | ⁿPr | PO | ⁿBu | 3 | C(CH₂O—)₄ | ⁿBu | PC | ASO/ |
| | | | | | | | | siRNA |
| 11 | propanoyl | — | complex^{†} | 3 | (—NH)C(CH₂O—)₃ | CO(CH₂)₁₀CO | complex^{†} | 3’-siRNA |
| 12 | ⁿPr | PS | ⁿBu | 3 | C(CH₂O—)₄ | ⁿBu | PS | ASO |
| 13 | ⁿPr | PO | ⁿBu | 3 | C(CH₂O—)₄ | ⁿBu | PS | ASO |
| 14 | ⁿPr | PS | ⁿBu | 3 | C(CH₂O—)₄ | ⁿBu | PO | ASO |
| 15 | ⁿPr | PG | ⁿBu | 3 | C(CH₂O—)₄ | ⁿPr | PO | ASO |
| 16 | ⁿPr | PG | ⁿBu | 3 | C(CH₂O—)₄ | bond | PG | ASO |
| 17 | ⁿPr | PS | ⁿBu | 3 | C(CH₂O—)₄ | ⁿBu | PS | siRNA |

*PO denotes phosphate, PS denotes thiophosphate, PG denotes a phosphoryl guanidine, and PNS denotes an N-sulfonylphosphoramidate; “—” denotes no group “P”
^{†}“dual” denotes an asymmetric splitter comprising two different arms; “complex” denotes a multipart moiety which is not shown in full

It will be appreciated that the following synthetic protocols are exemplary. Other routes for synthesising the compounds of the Examples will be apparent, e.g. based on the synthetic schemes set out above.

### Example 1: Cluster 1, PN-Guanidine Spacers

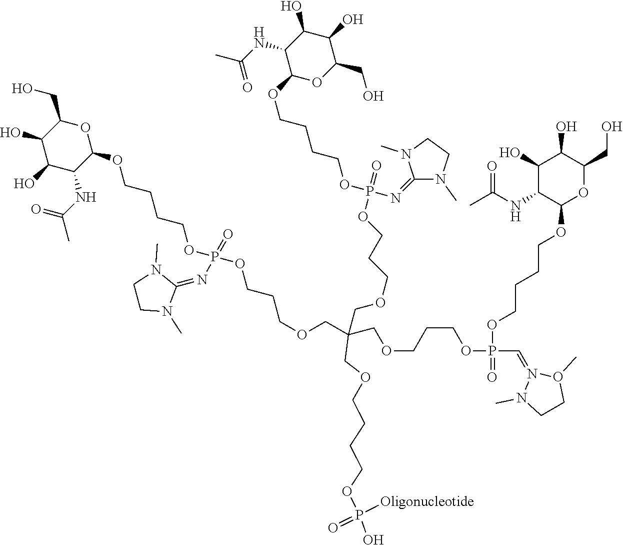

STEP 1: The ASO (MALAT1) and siRNA (ANGPTL3 and PPIB) oligonucleotide sequences were initially synthesized until final nucleotide on solid support using the general synthesis procedure above.

STEP 2: Cluster 1 was built on solid support starting from the 5′-OH of sequences prepared in step 1, as depicted in Scheme 1 above, using 2-azido-1,3-dimethylimidazolinium hexafluorophosphate for the Staudinger reaction. For siRNA compounds, guide and passenger strands were annealed following general procedure.

### Compound 3: Cluster 1 ASO (MALAT1)

UV purity 94%; m/z (ESI−; TOF) calcd 1779.323 (4−); found: 1779.602.
   Compound 4: Cluster 1 siRNA (ANGPTL3)
Passenger strand UV purity 86%; m/z (ESI+; TOF) calcd 1514.997 (6+); found: 1515.001.
   Compound 13: Cluster 1 siRNA (PPIB)
Passenger strand UV purity 91%; m/z (ESI−; TOF) calcd 2565.524 (3−); found: 2565.514.

### Example 2: Cluster 2, PN-Tosyl Spacers

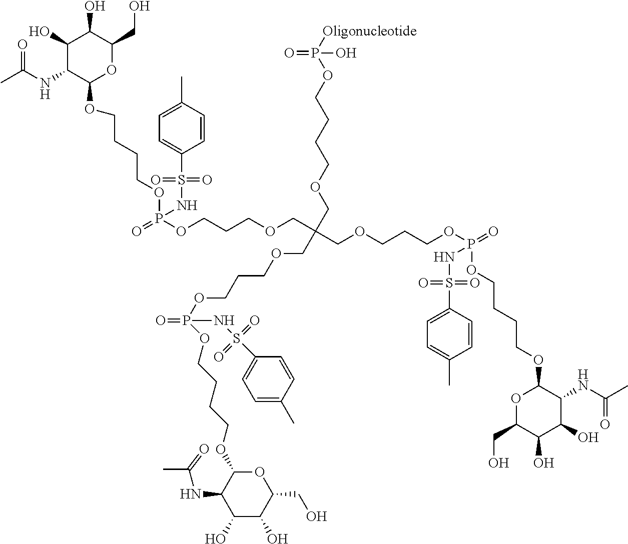

STEP 1: The siRNA (PPIB) oligonucleotide sequence was initially synthesized until final nucleotide on solid support using the general synthesis procedure above.

STEP 2: Cluster 2 was built on solid support starting from the 5′-OH of sequences prepared in step 1, as depicted in Scheme 1 above, using tosyl azide for the Staudinger reaction. Guide and passenger strands were subsequently annealed following general procedure.

Compound 5: Cluster 2 siRNA (ANGPTL3)
Passenger strand UV purity 92%; m/z (ESI+; TOF) calcd 1852.559 (5+); found: 1852.770.
   Compound 12: Cluster 2 siRNA (PPIB)
Passenger strand UV purity 92%; m/z (ESI−; TOF) calcd 2623.464 (3−); found: 2623.444.

### Example 3: Cluster 3, PN-Guanidine Spacers/C₁ Tether

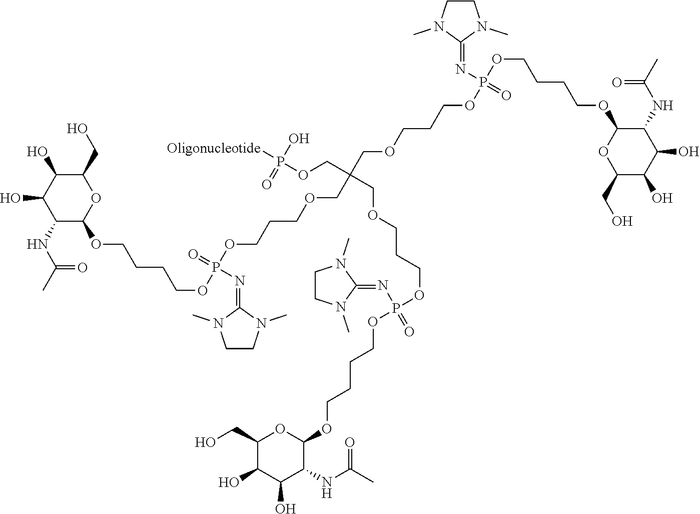

STEP 1: The ASO (MALAT1) oligonucleotide sequence was initially synthesized until final nucleotide on solid support using the general synthesis procedure above.

STEP 2: Cluster 3 was built on solid support starting from the 5′-OH of the sequence prepared in step 1, as described in Scheme 1 above, using 2-azido-1,3-dimethylimidazolinium hexafluorophosphate for the Staudinger reaction.

### Compound 14: Cluster 3 ASO (MALAT1)

UV purity 79%; m/z (ESI−; TOF) calcd 2348.748 (3−); found: 2348.725.

### Example 4: Cluster 4, PN-Guanidine Spacers/2× GalNAc

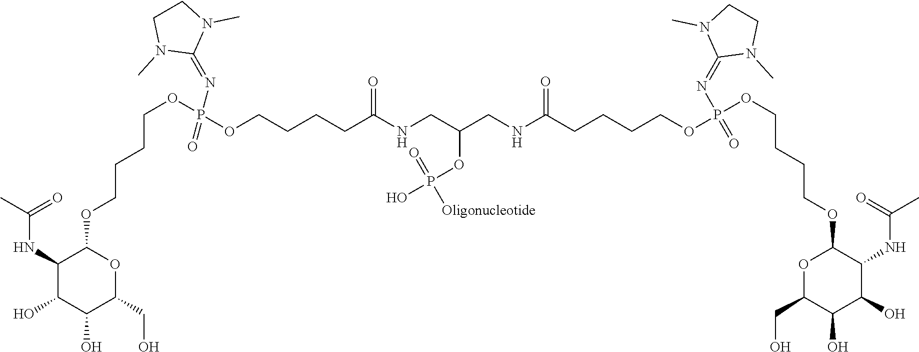

STEP 1: The ASO (MALAT1) oligonucleotide sequence was initially synthesized until final nucleotide on solid support using the general synthesis procedure above.

STEP 2: Cluster 4 was built on solid support starting from the 5′-OH of the sequence prepared in step 1, as described in Scheme 1 above, using 2-azido-1,3-dimethylimidazolinium hexafluorophosphate for the Staudinger reaction.

### Compound 15: Cluster 4 ASO (MALAT1)

UV purity 77%; m/z (ESI−; TOF) calcd 2192.014 (3−); found: 2192.012.

### Example 5: Cluster 5, PN-Guanidine Spacers/Sequential Double Splitter

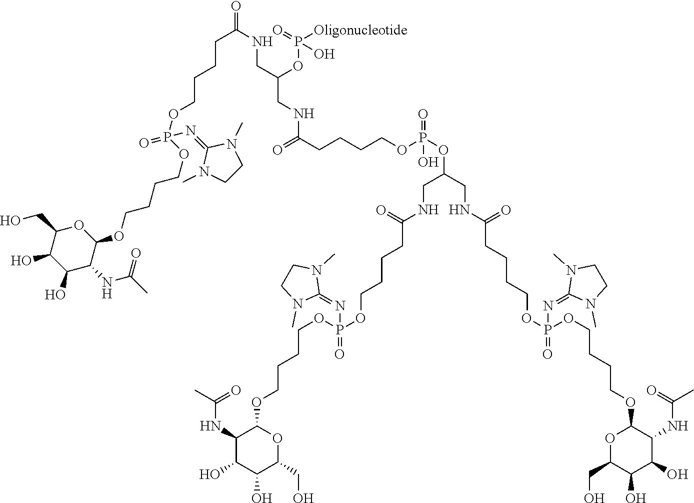

STEP 1: The ASO (MALAT1) oligonucleotide sequence was initially synthesized until final nucleotide on solid support using the general synthesis procedure above.

STEP 2: Cluster 5 was built on solid support starting from the 5′-OH of the sequence prepared in step 1, as described in Scheme 2 above, using 2-azido-1,3-dimethylimidazolinium hexafluorophosphate for the Staudinger reaction. Two asymmetric doublers were used sequentially using special procedure C to selectively remove DMTr and levulinyl protecting groups.

### Compound 16: Cluster 5 ASO (MALAT1)

UV purity 45%; m z (ESI−; TOF) calcd 2459.457 (3−); found: 2459.821.

### Example 6: Cluster 6, Phosphate Spacers/PN-Guanidine in Linker

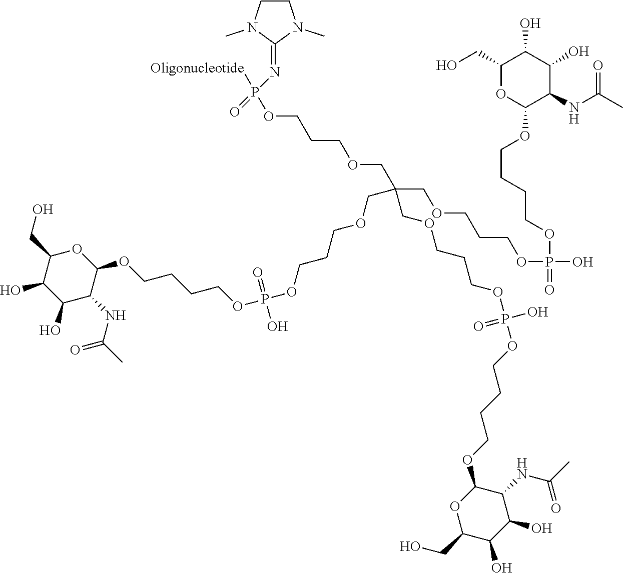

STEP 1: The ASO (MALAT1) oligonucleotide sequence was initially synthesized until final nucleotide on solid support using the general synthesis procedure above.

STEP 2: Cluster 6 was built on solid support starting from the 5′-OH of the sequence prepared in step 1, as described in Scheme 1 above, using 2-azido-1,3-dimethylimidazolinium hexafluorophosphate for the Staudinger reaction.

### Compound 17: Cluster 6 ASO (MALAT1)

UV purity 43%; m z (ESI−; quadrupole) calcd 1728.277 (3−); found: 1729.

### Example 7: Cluster 7, PN-Guanidine in Spacers and Linker

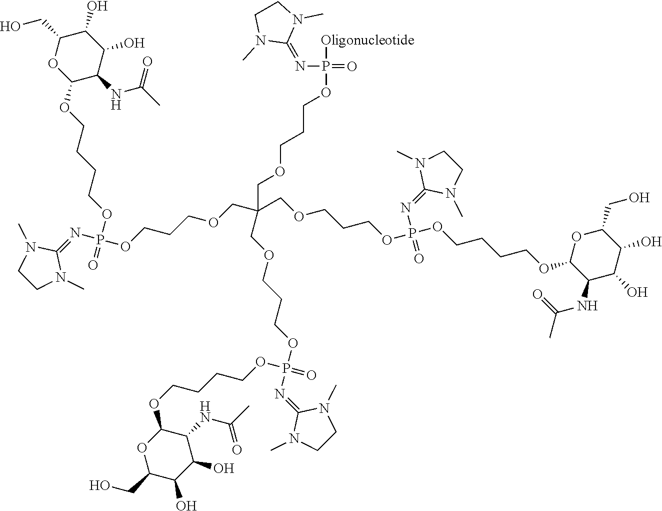

STEP 1: The ASO (MALAT1) oligonucleotide sequence was initially synthesized until final nucleotide on solid support using the general synthesis procedure above.

STEP 2: Cluster 7 was built on solid support starting from the 5′-OH of the sequence prepared in step 1, as described in Scheme 1 above, using 2-azido-1,3-dimethylimidazolinium hexafluorophosphate for the Staudinger reaction.

### Compound 18: Cluster 7 ASO (MALAT1)

UV purity 80%; m/z (ESI−; TOF) calcd 1799.591 (4−); found: 1800.372.

### Example 8: Cluster 8, PN-Guanidine Spacers and Diaminopropanol Tether

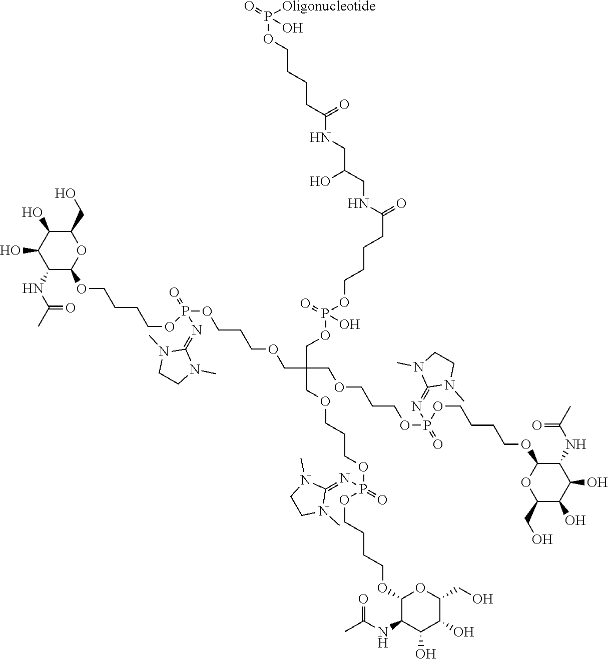

STEP 1: Cluster 8 was built on universal Controlled-Porosity Glass solid support, as described in Scheme 2 above, using 2-azido-1,3-dimethylimidazolinium hexafluorophosphate for the Staudinger reaction.

STEP 2: The MALAT1 ASO sequence was subsequently synthesized on the remaining unfunctionalized alcohol of the asymmetric doubler, as described in special procedure C.

### Compound 19: Cluster 8 ASO (3′ Linked MALAT1)

UV purity 97%; m/z (ESI−; TOF) calcd 1849.344 (4−); found: 1849.375.

### Example 9: Control Cluster 1, Triantennary/Phosphate Spacers

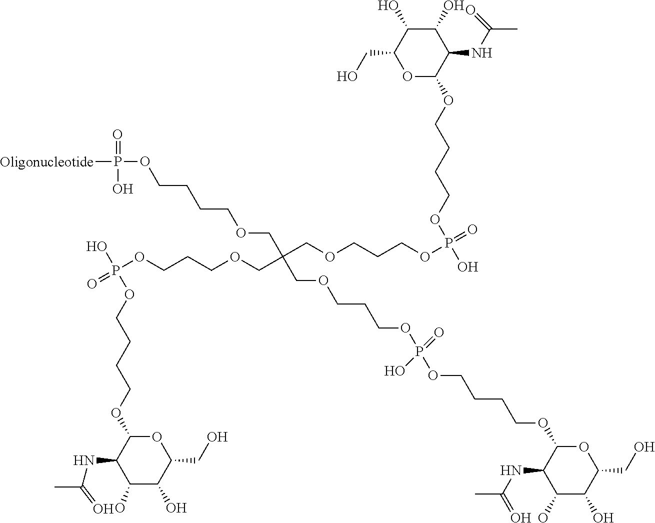

STEP 1: The ASO (MALAT1) and siRNA (passenger strand for PPIB) oligonucleotide sequences were initially synthesized until final nucleotide on solid support using the general synthesis procedure above.

STEP 2: Control cluster 1 was built on solid support starting from the 5′-OH of sequences prepared in step 1, as described in Scheme 1, using standard oxidation conditions. For siRNA compounds, guide and passenger strands were subsequently annealed following general procedure.

Compound 1 Control Cluster 1 siRNA (ANGPTL3)
Passenger strand UV purity 94%; m/z (ESI+; TOF) calcd 1760.744 (5+); found: 1760.747.

### Compound 7: Control Cluster 1 ASO (MALAT1)

UV purity 86%; m/z (ESI−; TOF) calcd 2277.683 (3−); found: 2277.679.
   Compound 11: Control Cluster 1 siRNA (PPIB)
Passenger strand UV purity 85%; m/z (ESI−; TOF) calcd 2470.440 (3−); found: 2470.431.

### Example 10: Control Cluster 2, Triantennary/Amide Spacers and Linker

Control cluster 2 is a tri-antennary GalNAc cluster having amide spacers and a linker of the type described herein. It carries a single oligonucleotide, i.e. the cluster has four arms in total.

STEP 1: The ASO (MALAT1) oligonucleotide sequence was initially synthesized until final nucleotide on solid support using the general synthesis procedure above.

STEP 2: Control cluster 2 was built starting from the 5′-OH of the sequence prepared in step 1, as described in Scheme 3 above, using special procedure A.

### Compound 2: Control Cluster 2 ASO (MALAT1)

UV purity 99%. m/z (ESI−; TOF) calcd 1710.829 (4−); found: 1710.610.

### Example 11: Control Cluster 3, Triantennary/Amide Spacers and Prolinol Linker

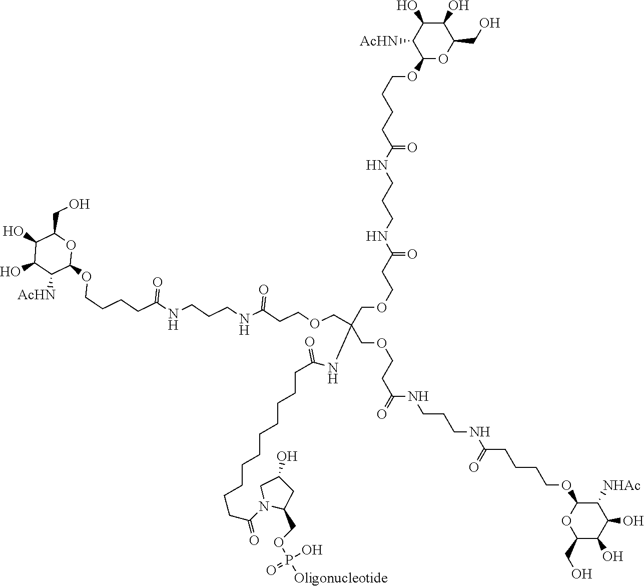

Passenger strand for siRNA (ANGPTL3) was synthesized on GalNAc-loaded solid support according to special procedure B. The alcohol on the pyrrolidine serves as attachment point to the Controlled-Porosity Glass solid support.

Compound 6 Control Cluster 3 siRNA-3′ Linked (ANGPTL3)
Passenger strand UV purity 92%; m/z (ESI−; quadrupole) calcd 1814.408 (5−); found: 1815.

### Example 12: Control Cluster 4, Triantennary/PS Spacers and Linker

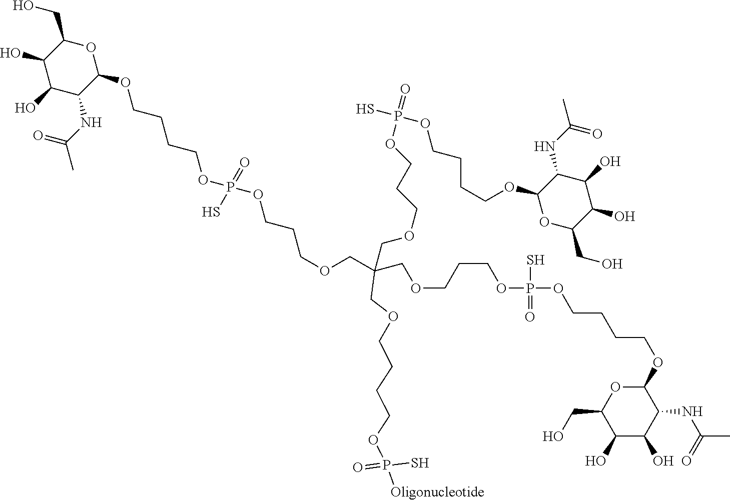

STEP 1: The ASO (MALAT1) oligonucleotide sequence was initially synthesized until final nucleotide on solid support using the general synthesis procedure above.

STEP 2: Control cluster 4 was built on solid support starting from the 5′-OH of the sequence prepared in step 1, as described in Scheme 1 above, using standard thiolation conditions.

### Compound 8: Control Cluster 4 ASO (MALAT1)

UV purity 92%; m/z (ESI−; TOF) calcd 2298.986 (3−); found: 2298.978.

### Example 13: Control Cluster 5, Triantennary/Phosphate Spacers and PS Linker

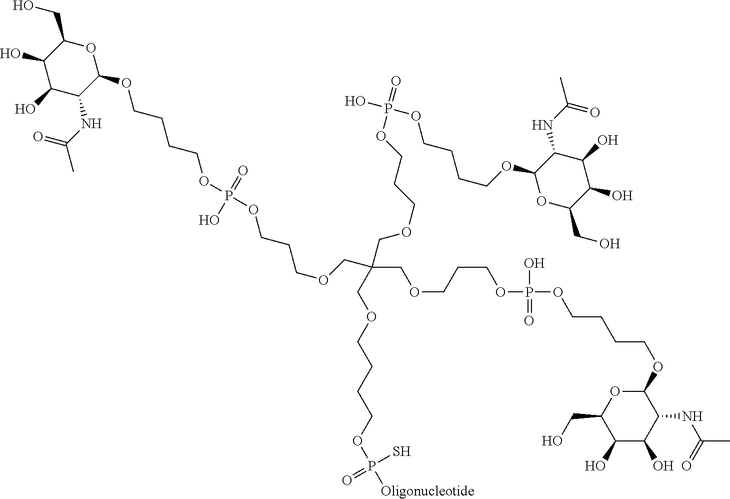

STEP 1: The ASO (MALAT1) oligonucleotide sequence was initially synthesized until final nucleotide on solid support using the general synthesis procedure above.

STEP 2: Control cluster 5 was built on solid support starting from the 5′-OH of the sequence prepared in step 1, as described in Scheme 1 above, using standard thiolation and oxidation conditions.

### Compound 9: Control Cluster 5 ASO (MALAT1)

UV purity 91%; m/z (ESI−; TOF) calcd 2283.008 (3−); found: 2283.005.

### Example 14: Control Cluster 6, Triantennary/PS Spacers and Phosphate Linker

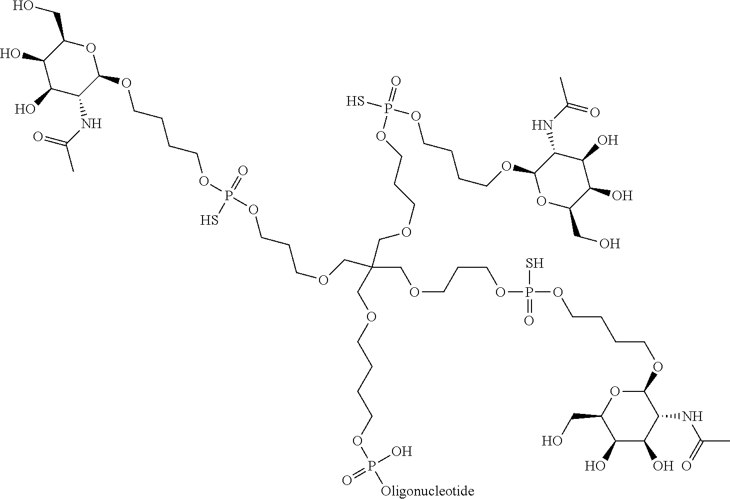

STEP 1: The ASO (MALAT1) oligonucleotide sequence was initially synthesized until final nucleotide on solid support using the general synthesis procedure above.

STEP 2: Control cluster 6 was built on solid support starting from the 5′-OH of the sequence prepared in step 1, as described in Scheme 1 above, using standard oxidation and thiolation conditions.

### Compound 10: Control Cluster 6 ASO (MALAT1)

UV purity 93%; m/z (ESI−; TOF) calcd 2293.660 (3−); found: 2293.659.

### Example 15: Cluster 9, PN-Guanidine Spacers

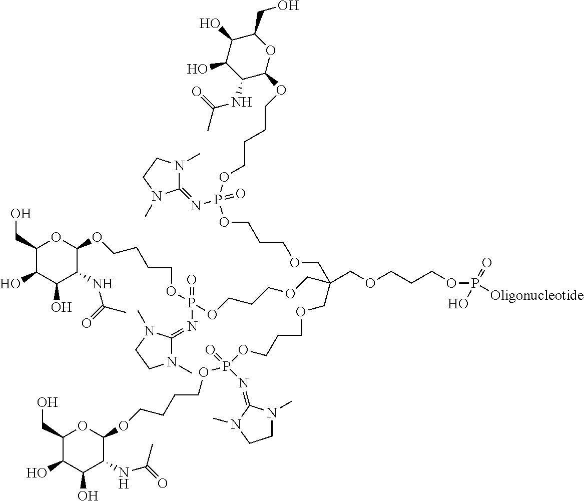

STEP 1: The ASO (MALAT1) oligonucleotide sequence was initially synthesized until final nucleotide on solid support using the general synthesis procedure above.

STEP 2: Cluster 9 was built on solid support starting from the 5′-OH of the sequence prepared in step 1, as described in Scheme 1 above, using azido-1,3-dimethylimidazolinium hexafluorophosphate for the Staudinger reaction.

### Compound 20: Cluster 9 ASO (MALAT1)

UV purity 96%; m/z (ESI−; TOF) calcd 1776.827 (4−); found: 1776.833.

### Example 16: Cluster 10, PN-Guanidine in Spacers and Linker

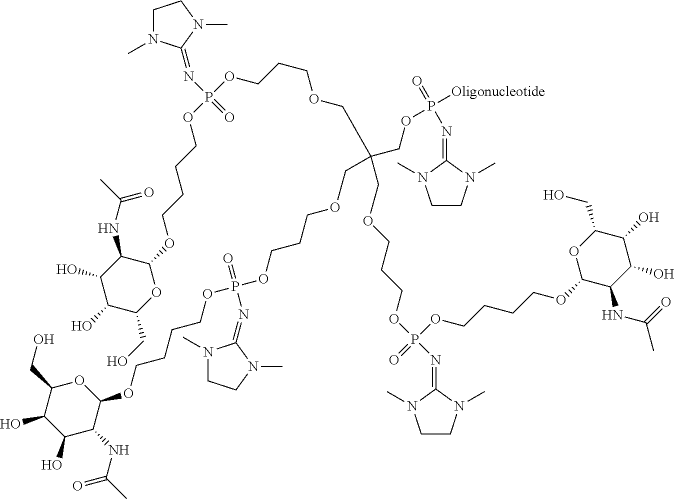

STEP 1: The ASO (MALAT1) oligonucleotide sequence was initially synthesized until final nucleotide on solid support using the general synthesis procedure above.

STEP 2: Cluster 10 was built on solid support starting from the 5′-OH of the sequence prepared in step 1, as described in Scheme 4 above, using 2-azido-1,3-dimethylimidazolinium hexafluorophosphate for the Staudinger reaction.

### Compound 21: Cluster 10 ASO (MALAT1)

UV purity 99%; m/z (ESI−; TOF) calcd 1786.088 (4−); found: 1786.080.

### Example 17: Control Cluster 7, Triantennary/Thiophosphate Spacers

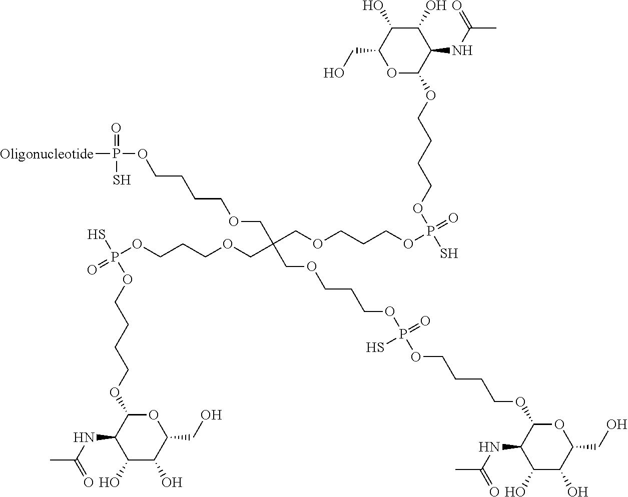

STEP 1: The siRNA (passenger strand for PPIB, SEQ ID NO: 4) oligonucleotide sequence was initially synthesized until final nucleotide on solid support using the general synthesis procedure above.

STEP 2: Control cluster 7 was built on solid support starting from the 5′-OH of sequences prepared in step 1, as described in Scheme 1, using standard thiolation conditions. For siRNA compounds, guide (SEQ ID NO: 5) and passenger strands were subsequently annealed following general procedure.

Compound 22 Control Cluster 7 siRNA (PPIB)
Passenger strand UV purity 91%; m/z (ESI+; TOF) calcd 1869.563 (4−); found: 1869.547.

### Examples 18 to 21—Biological Assays

Several assays were performed to assess the activity of compounds of the Examples in both in vitro and in vivo systems.

### Example 18: Acute Liver Toxicity (Caspase Activation) Assay

A caspase assay was performed using the following protocol:

### Day 1

### Preparation of Lipofectamine Solution

1. Prepare 0.5% Lipofectamine solution by mixing Lipofectamine from stock (Lipofectamine 2000, Invitrogen) with Opti-MEM I Reduced Serum medium (Gibco). Invert the lipofectamine stock solution before use. Volumes with 5% excess volume.

| Regent | 2 plates | 4 plates | 8 plates | 12 plates |
|---|---|---|---|---|
| Lipofectamine | 40 μL | 80 μL | 161 μL | 242 μL |
| OptiMem | 8064 μL | 16128 μL | 32256 μL | 48384 μL |

2. Dispense the Lipofectamine solution to 384 cell assay ready plates plate(s) using a small multidrop combi cassette (med speed, 10 μL/well) and incubate for at least 30 minutes prior to adding the cells.

### Dispensing and HepG2 C3A

3. Cryo-vial(s) of HepG2 C3A is quickly thawed in 37° C. water. One cryo-vial is enough for ˜4 cell plates assuming 5 million cells per vial and >70% viable cells.
4. Thawed cells are transferred to a 15 mL falcon tube(s), warm DMEM media is added to the cells (10 mL).
5. Cells are centrifuged at 100 g for 5 minutes, resulting supernatant is decanted.
6. Fresh warm DMEM media is added (5 mL) to the resulting cell pellet.
7. Cells are sheared and mixed by pipetting up and down for at least 5 times to ensure a single cell suspension. Cell suspension is pooled if more than one cryo-vial is used.
8. Density of the cell suspension is determined using the Vi-CELL BLU cell counter.
9. Cell suspension is diluted to 2E5 cells/mL in DMEM media.
10. Cell suspension is added to assay plate using a small multidrop combi cassette (medium speed, 10 μL/well, 2000 cells per well).
11. Cell assay plate is incubated for 24 hours in 37° C.

### Day 2

### Dispensing Caspase Assay and Reading Plate

12. Caspase 3/7 assay solution is prepared by mixing Caspase-Glo 3/7 buffer and lyophilized Caspase-Glo 3/7 (equilibrated to room temperature). Leftover caspase can be stored for at least four weeks at −20° C.
13. Add Caspase-Glo solution using a small multidrop combi cassette to cell assay plate(s) (High speed, 10 μL/well).
14. Incubate cell assay plates at room temperature placed on shaker at 700 rpm for >30 minutes. Signal should be stable up to 4 hours according to manufacturer.
15. Read plate(s) in Pherastar (or equivalent plate reader) using luminescence.

The results of the assay for the tested compounds are shown in Table 1 below (a compound is assessed as being non-toxic when the caspase activation level does not reach more than 30% of that of a toxic control compound at the highest tested concentration):

TABLE 1
Results of caspase
(liver toxicity) assay

| Compound No. | Toxicity flag |
|---|---|
| 8 | non-toxic |
| 14 | non-toxic |
| 15 | non-toxic |
| 16 | non-toxic |
| 17 | non-toxic |
| 18 | non-toxic |
| 20 | non-toxic |
| 21 | non-toxic |

The data in Table 1 indicate that all tested compounds were assessed as being non-toxic by the caspase assay.

### Example 19: ANGPTL3 and PPIB Knockdown in Human Primary Hepatocytes

An assay was carried out to assess the ability of the siRNA (ANPTL3 and PPIB) constructs to yield knockdown gene expression in human primary hepatocytes.

The following cell culture protocol was used:

Human hepatocytes were obtained from a commercial supplier (BioIVT, QNT lot) and cultured in rat tail collagen I coated plates. The hepatocytes were maintained in William's E Medium, supplemented with hepatocyte supplemented media and 5C supplements (DAPT, SB431542, Forskolin, IWP2 and LDN193189). The cells were cultured at 37° C. in a humidified incubator with 5% CO2. Next after plating (Day 1), cells were treated with different siRNA conjugates and PBS in maintenance media. After 24 h of treatment, media was changed and cells were harvested on Day 4 (72 hr from start of the treatment) for qPCR. Total RNA was extracted according to standard protocol setup in the lab. cDNA synthesis and qPCR were also run according to standard validated protocol. PPIB, ANGPLT3 and GAPDH TaqMan primers were purchased from Thermo Fischer.

The results of the assay for exemplary compounds are shown in Table 2 below:

TABLE 2
Knockdown efficiency in
human primary hepatocytes

| Compound No. | IC₅₀ (nM) |
|---|---|
| 1 (ANGPTL3) | 40% knockdown |
| | at 1 μM |
| 4 (ANGPTL3) | 40% knockdown |
| | at 1 μM |
| 6 (ANGPTL3) | 200 |
| 11 (PPIB) | 24 |
| 12 (PPIB) | 18 |
| 13 (PPIB) | 18 |

The data in Table 2 indicate that all tested compounds are capable of knocking down gene expression in human primary hepatocytes.

### Example 20: PPIB and MALAT1 Knockdown in HEK293 Cells

An assay was carried out to assess the ability of the siRNA (PPIB) and ASO (MALAT1) constructs to knockdown gene expression in HEK293 cells overexpressing ASGPR.

Cryopreserved HEK293s-ASGPR1 cells were thawed and seeded directly in assay-ready plates containing siRNA or ASO compounds as PBS serial dilutions (Day 0). Cells were incubated for 48 h at 37° C., 5% CO₂ and 95% humidity. Two days after plating (Day 2), cells were lysed and RNA was extracted according to standard protocol. cDNA synthesis and qPCR were also run according to standard validated protocol. PPIB, MALAT1 and RACK1 TaqMan primers were purchased from Thermo Fisher.

The results of the assay for the tested compounds are shown in Table 3 below (geometric mean±standard deviation is shown where repeat measurements were taken):

TABLE 3
PPIB and MALAT1
knockdown in HEK293 cells

| Compound No. | IC₅₀ (nM) |
|---|---|
| 3 (MALAT1) | 110 ± 46 |
| 7 (MALAT1) | >10,000 |
| 8 (MALAT1) | 340 |
| 9 (MALAT1) | 79 ± 4 |
| 10 (MALAT1) | 133 |
| 11 (PPIB) | 720 |
| 13 (PPIB) | 200 |
| 14 (MALAT1) | 620 |
| 15 (MALAT1) | 1300 |
| 16 (MALAT1) | 37.0 |
| 17 (MALAT1) | 270 |
| 18 (MALAT1) | 68.0 |

The assay was performed with compounds 20 and 21 and a further repeat carried out for compounds 3 and 17. The results of these assays are shown in Table 3B below (geometric mean±standard deviation is shown where repeat measurements were taken; these values were calculated based on replicate(s) in addition to the data represented above in Table 3):

TABLE 3B
MALAT1 knockdown
in HEK293 cells

| Compound No. | IC₅₀ (nM) |
|---|---|
| 3 (MALAT1) | 104 ± 28 |
| 17 (MALAT1) | 352 ± 95 |
| 20 (MALAT1) | 150 |
| 21 (MALAT1) | 68.0 |

The data in Table 3 and Table 3B indicate that the test compounds were active, with some having an activity surpassing that of the control compounds.

### Example 21: Mouse In Vivo Study for Evaluation of Exposure and Knockdown Efficiency in Liver, Kidney, Heart and Lungs

An in vivo study was carried out in mice (N=4 or 5) to evaluate exposure and knockdown efficiency of the compounds in liver, kidney, heart and lungs.

### ANGPTL3 Knockdown

Compounds 1, 4 and 6, all targeting Angptl3 were investigated in vivo in female C57 BL6/J mice (8-10 weeks old with bodyweight 20-30 g) using a single subcutaneous dose of 2 mg/kg (N=5 per group). An additional control group dosed with PBS was also included in the study (N=5). Blood samples were drawn pre-dose and 7, 21, 28 and 42 days after dosing, as well as after animals were euthanized on day 49 after dosing. The ANGPTL3 protein concentration in circulation was measured by ELISA (Catalog Number MANL30; R&D Systems). Heart, liver, kidney, and lung tissues were sampled on day 49 for measurement of concentration of the antisense strand using liquid chromatography-mass spectrometry. The Angptl3 mRNA levels in liver samples were measured by qPCR and Angptl3 mRNA levels were normalized over the geometric mean of Actb and Malat1 mRNA levels.

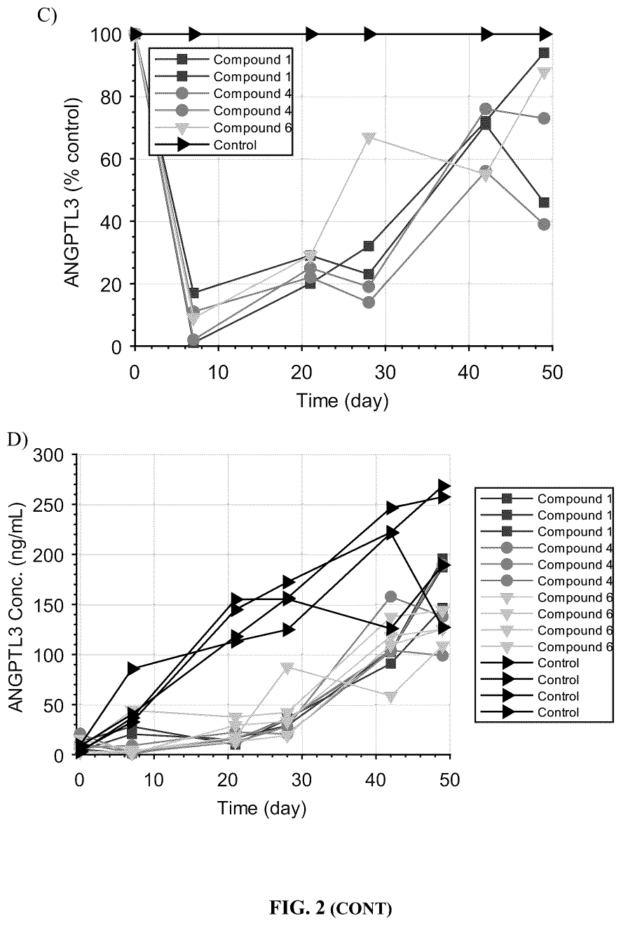

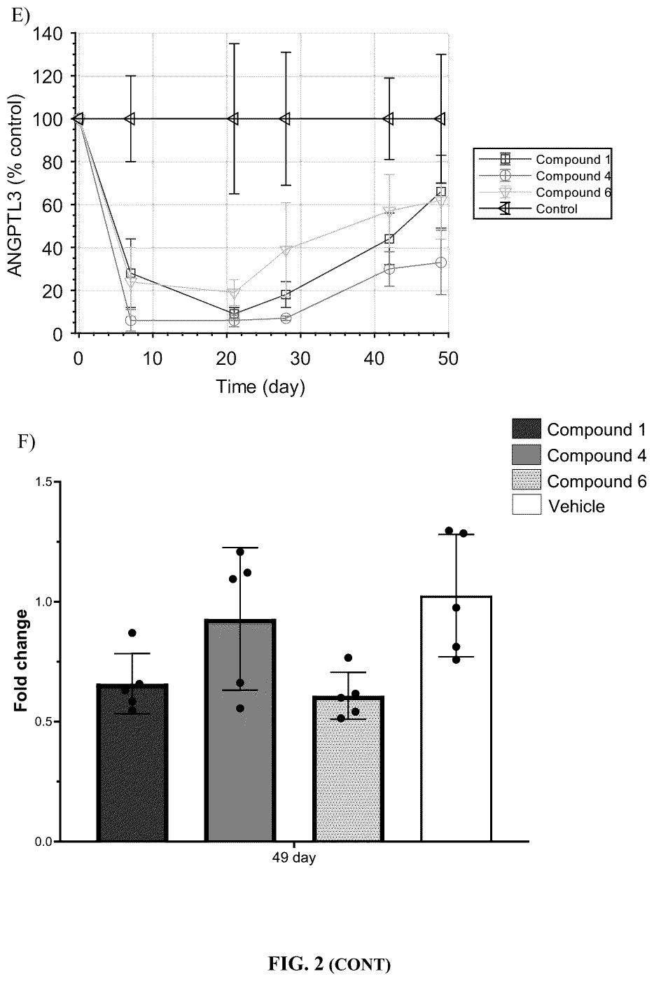

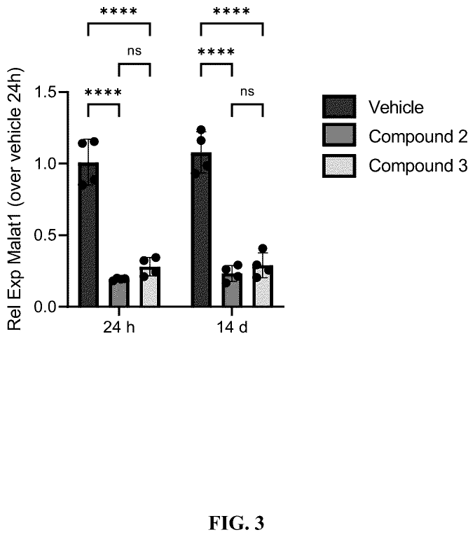

The primary readout in form of ANGPTL3 protein data indicated high variability in baseline levels, ranging two orders of magnitude from 2.6 ng/mL to −260 ng/mL. The distribution was approximately bimodal, and it was decided to analyze the data in two parts (FIG. 2A). The first part included data from 6 animals with baseline ANGPTL3 protein around 200 ng/mL corresponding to the typical level observed in historical data for this in vivo model. The second part included the remaining data from 14 animals with a baseline ANGPTL3 protein of ≤21 ng/mL. The data of the first part show a pronounced and transient decline in ANGPTL3 protein for all treated groups, and a less pronounced and transient decline for the negative control group (FIG. 2B). The baseline and control-corrected data indicate a more than 80% reduction of ANGPTL3 protein after one week and a slow return towards baseline with reductions between 5 and 60% for individual animals at week 7 (FIG. 2C). The data of the second part show a steady increase in the negative control group towards the normal value of about 200 ng/mL ANGPTL3 protein. The treated groups also show increased ANGPTL3 protein over time but at a much slower pace and magnitude compared to the control group (FIG. 2D). The baseline and control-corrected data indicate a more than 80% reduction of ANGPTL3 protein after two weeks and a slow return towards baseline with group mean reductions between 40 and 70% at week 7 (FIG. 2E).

The secondary readout tissue concentration data indicate low tissue levels, something that was expected due to the long study duration. Liver concentrations were similar between the substances: 11.4 (SE 1.9) nmol/kg, 8.60 (SE 1.6) nmol/kg, 9.51 (SE 0.82) nmol/kg for compound 1, 4 and 6, respectively. In the other tissues, data were below the lower limit of quantification (LLOQ; 5.86 nmol/kg), expect for kidney data of compound 6 which were above the LLOQ in four out of five animals (8, 8, 13, 8.3 and 12 nmol/kg in those four animals).

For the other secondary readout, expression level of Angptl3 mRNA in liver homogenate, data indicate up to 30% knockdown of the mRNA levels for the treated groups compared to the control group. Compound 4 showed less knockdown compared to Compound 1 and Compound 6, but also greater variability.

Despite the variability in baseline ANGPTL3 protein, the data altogether indicate that Compounds 1, 4 and 6 result in similar reduction of ANGPTL3 protein, and this is also supported by the similarity in liver concentration data at termination. The slight difference between the three compounds in expression level of Angptl3 mRNA in liver homogenate deviates from the overall pattern and can likely be attributed to natural variability within a dynamic range of the measured variable (which is returning to baseline around the time of measurement).

### MALAT1 Knockdown

Compounds 2 and 3, both targeting Malat1 were investigated in vivo in female C57 BL6/J mice (8-10 weeks old with bodyweight 20-30 g) using a single subcutaneous dose of 2 mg/kg (N=8 per group). An additional control group dosed with PBS was also included in the study (N=8). For each group, four animals were euthanized 24 h after dosing, and the remaining 4 animals 14 days after dosing, and tissue samples of liver, kidney, heart and lung were collected. Tissue concentrations were measured by liquid chromatography-mass spectrometry. The Malat1 mRNA levels in liver samples was measured by qPCR and Malat1 levels were normalized over the geometric mean of Actb, Hprt and Tbp.

Liver concentrations were similar between compounds: 4.66 (SE 0.61) μM, 3.10 (SE 0.46) μM for compound 2 and 3 at 24 h, and 0.290 (SE 0.052) μM, 0.172 (SE 0.010) μM for compound 2 and 3 at 14 days. Both Compound 2 and Compound 3 significantly suppressed Malat1 in the liver, and at a similar magnitude.

It is to be understood that while the disclosure has been described in conjunction with the above embodiments, that the foregoing description and examples are intended to illustrate and not limit the scope of the disclosure. Other aspects, advantages, and modifications within the scope of the disclosure will be apparent to those skilled in the art to which the disclosure pertains.

In addition, where features or aspects are described in terms of Markush groups, those skilled in the art will recognize that such features or aspects are also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

1. A nucleic acid delivery agent, which is a compound, or a pharmaceutically acceptable salt thereof, wherein the compound comprises a nucleic acid and at least one moiety having the structure of Formula (I): 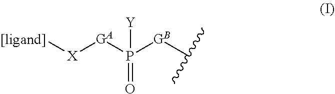
wherein:
X is selected from optionally substituted —(C₁-C₁₀)alkylene-, —(C₁-C₁₀)alkenylene-, and *—[(CH₂)₂O]ₒ(CH₂)₂-j wherein * denotes the point of attachment of the group to G^{A}, and wherein o is an integer selected from 1, 2, and 3;
G^{A} and G^{B} are each independently selected from O and NH, provided that at least one of G^{A} and G^{B} is O;
Y is a —NH-sulfonyl group or a guanidine-containing moiety; and
[ligand] denotes a monosaccharide.

2. (canceled)

3. The nucleic acid delivery agent of claim 1, wherein the moiety having the structure of Formula (I) is a moiety having the structure of Formula (II): 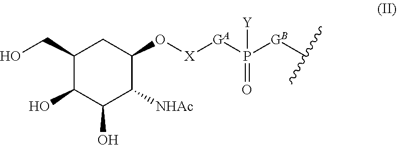
wherein X, G^{A} G^{B}, and Y are as defined in claim 1.

4. The nucleic acid delivery agent of claim 1, wherein X is selected from optionally substituted —(C₃-C₆)alkylene- and *—[(CH₂)₂O]ₒ(CH₂)₂—; wherein * denotes the point of attachment of the group to G^{A}, and wherein o is an integer selected from 1, 2, and 3.

5. The nucleic acid delivery agent of claim 1, wherein X is —(CH₂)ₘ— wherein m is an integer selected from 3, 4, 5, and 6; or wherein X is —CH₂CH₂OCH₂CH₂—.

6. The nucleic acid delivery agent of claim 1, wherein G^{A} and G^{B} are each O.

7.-8. (canceled)

9. The nucleic acid delivery agent of claim 1, wherein Y is a guanidine-containing moiety.

10. A compound, or a pharmaceutically acceptable salt thereof, wherein the compound comprises one or more moieties having a structure independently selected from Formula (I) and Formula (II): 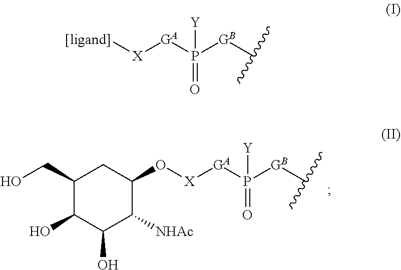
and one or more cargo moieties, wherein:
each cargo moiety comprises a nucleic acid;
X is selected from optionally substituted —(C₁-C₁₀)alkylene-, —(C₁-C₁₀)alkenylene-, and *—[(CH₂)₂O]ₒ(CH₂)₂—; wherein * denotes the point of attachment of the group to G^{A}, and wherein o is an integer selected from 1, 2, and 3;
G^{A} and G^{B} are each independently selected from O and NH, provided that at least one of G^{A} and G^{B} is O;
Y is a —NH-sulfonyl group or a guanidine-containing moiety; and
[ligand] denotes a monosaccharide.

11. The compound, or the pharmaceutically acceptable salt thereof, of claim 10, wherein the compound has the structure of Formula (III): 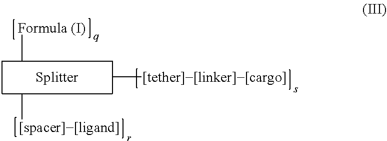
wherein each Formula (I) group has a structure independently selected from Formula (I) as defined in claim 10, and wherein:
q is an integer selected from 1, 2, 3, and 4;
r is an integer selected from 0, 1, 2, and 3;
s is an integer selected from 1 and 2;
[ligand] in each case independently denotes a monosaccharide;
[spacer] in each case independently denotes a moiety comprising a chain of atoms that serves to link the ligand to the splitter;
Splitter denotes a moiety having at least 2 points of attachment for the Formula (I), [spacer]-[ligand], and [tether]-[linker]-[cargo] moieties where present;
[tether] and [linker] taken together in each case independently denote a moiety comprising a chain of atoms that serves to link the cargo to the splitter; and
[cargo] denotes the cargo moiety.

12. The compound, or the pharmaceutically acceptable salt thereof, of claim 11, wherein the ligand in each case is GalNAc.

13. The compound, or the pharmaceutically acceptable salt thereof, of claim 11, wherein the sum of q, r, and s is 3, 4, or 5.

14. The compound, or the pharmaceutically acceptable salt thereof, of claim 11, wherein each spacer where present independently has a structure which comprises 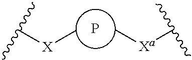
wherein:
X is selected from optionally substituted —(C₁-C₁₀)alkylene-, —(C₁-C₁₀)alkenylene-, and *—[(CH₂)₂O]ₒ(CH₂)₂—; wherein * denotes the point of attachment of the group to G^{A}, and wherein o is an integer selected from 1, 2, and 3;
X^{a} is optionally substituted —(CH₂)ₜ— or —C(O)(CH₂)ᵤ*; wherein * denotes the point of attachment to the group “P”; and t and u are integers independently selected from 1, 2, 3, 4, 5 and 6; and
group “P” is selected from phosphate, thiophosphate, and phosphoramidate.

15. The compound, or the pharmaceutically acceptable salt thereof, of claim 14, wherein X is —(CH₂)ₘ— or *—[(CH₂)₂O]ₒ(CH₂)₂—; wherein * denotes the point of attachment of the group to “P”; m is an integer selected from 3, 4, and 5; and o is an integer selected from 1, 2, and 3.

16. The compound, or the pharmaceutically acceptable salt thereof, of claim 11, wherein the Splitter comprises the group: 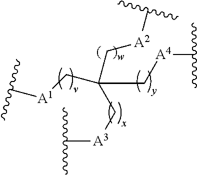
wherein:
A¹, A², A³, and A⁴ are each independently selected from —O—, —S—, —NH—, —CH₂—, and —C(O)—; and
v, w, x, and y are each an integer independently selected from 0, 1, 2 and 3.

17. The compound, or the pharmaceutically acceptable salt thereof, of claim 16, wherein each of A¹, A², A³, and A⁴ is —O—.

18. The compound, or the pharmaceutically acceptable salt thereof, of claim 16, wherein each of v, w, x, and y is 1.

19. The compound, or the pharmaceutically acceptable salt thereof, of claim 16, wherein the Formula (I) and [spacer]-[ligand] moieties when present are connected to the splitter via A¹, A², and A³, and the [tether]-[linker]-[cargo] moiety is connected to the splitter via A⁴.

20. The compound, or the pharmaceutically acceptable salt thereof, of claim 10, wherein each cargo moiety is independently selected from an antisense oligonucleotide, an immunostimulatory oligonucleotide, a decoy oligonucleotide, a splice altering oligonucleotide, a splice-switching oligonucleotide, a triplex forming oligonucleotide, a siRNA, a saRNA, a microRNA, a microRNA mimic, an anti-miR, a double stranded RNA, a single stranded RNA, a ribozyme, an aptamer, a spiegelmer, a CRISPR oligonucleotide, and a G-quadruplex.

21. The compound, or the pharmaceutically acceptable salt thereof, of claim 20, wherein each cargo moiety is an antisense oligonucleotide, or wherein each cargo moiety is a siRNA.

22.-30. (canceled)

31. A compound, or a pharmaceutically acceptable salt thereof, wherein the compound has the structure of Formula (VI): 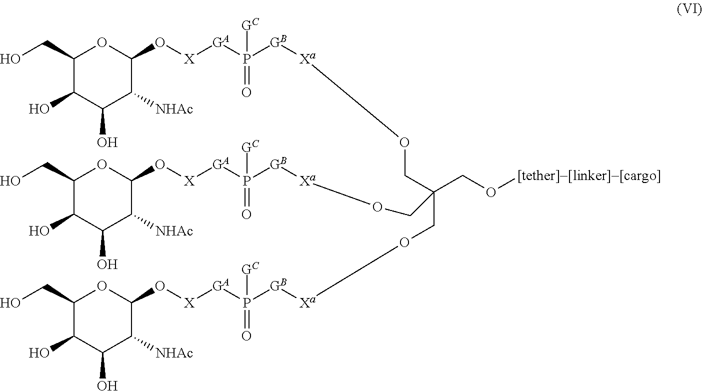
wherein:
X is selected from optionally substituted —(C₁-C₁₀)alkylene-, —(C₁-C₁₀)alkenylene-, and *—[(CH₂)₂O]ₒ(CH₂)₂—; wherein * denotes the point of attachment of the group to G^{A}, and wherein o is an integer selected from 1, 2, and 3;
G^{A} and G^{B} are each independently selected from O and NH, provided that at least one of G^{A} and G^{B} is O;
G^{C} in each case is independently selected from Y and Z;
Y is a —NH-sulfonyl group or a guanidine-containing moiety;
Z in each case is independently selected from —SH and —OH;
X^{a} is optionally substituted —(CH₂)ₜ— or —C(O)(CH₂)ᵤ—; wherein t and u are integers independently selected from 1, 2, 3, 4, 5, and 6;
[tether] and [linker] taken together in each case independently denote a moiety comprising a chain of atoms that serves to link the [cargo] to the oxygen atom to which the [tether] is attached; and
[cargo] denotes a cargo moiety comprising a nucleic acid.

32. The compound, or the pharmaceutically acceptable salt thereof, of claim 31, wherein G^{A} and G^{B} in each case are O, and wherein all three of the G^{C} groups are independently selected from Y.

33. The compound, or the pharmaceutically acceptable salt thereof, of claim 31, wherein X in each case is independently —(CH₂)ₘ— wherein each m is independently an integer selected from 3, 4, 5, and 6.

34. The compound, or the pharmaceutically acceptable salt thereof, of claim 31, wherein X^{a} in each case is independently —(CH₂)— wherein each t is independently an integer selected from 2, 3, and 4.

35. The compound or pharmaceutically acceptable salt thereof of claim 31, wherein the [tether]-[linker]-[cargo] moiety is selected from:
i) 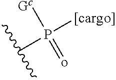
wherein G^{C} is Z;
ii) 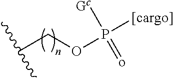
wherein n is selected from 3 and 4, and G^{C} is Z or Y; and
iii) 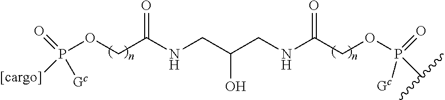
wherein n is 4, and G^{C} is Z.

36.-45. (canceled)

46. A compound selected from: 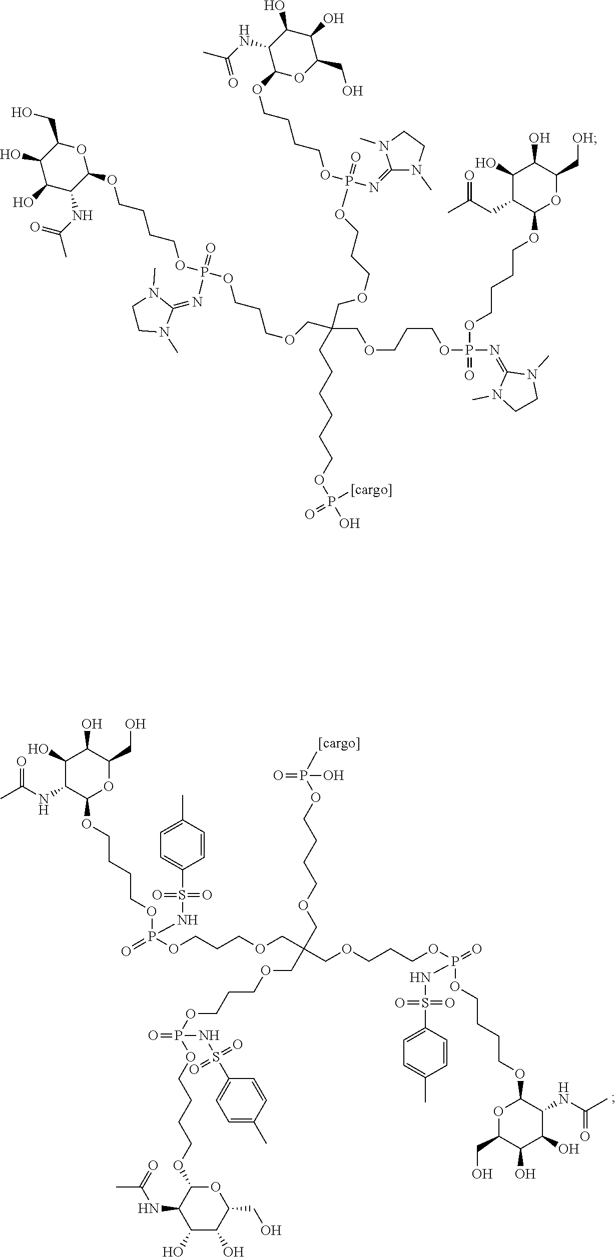 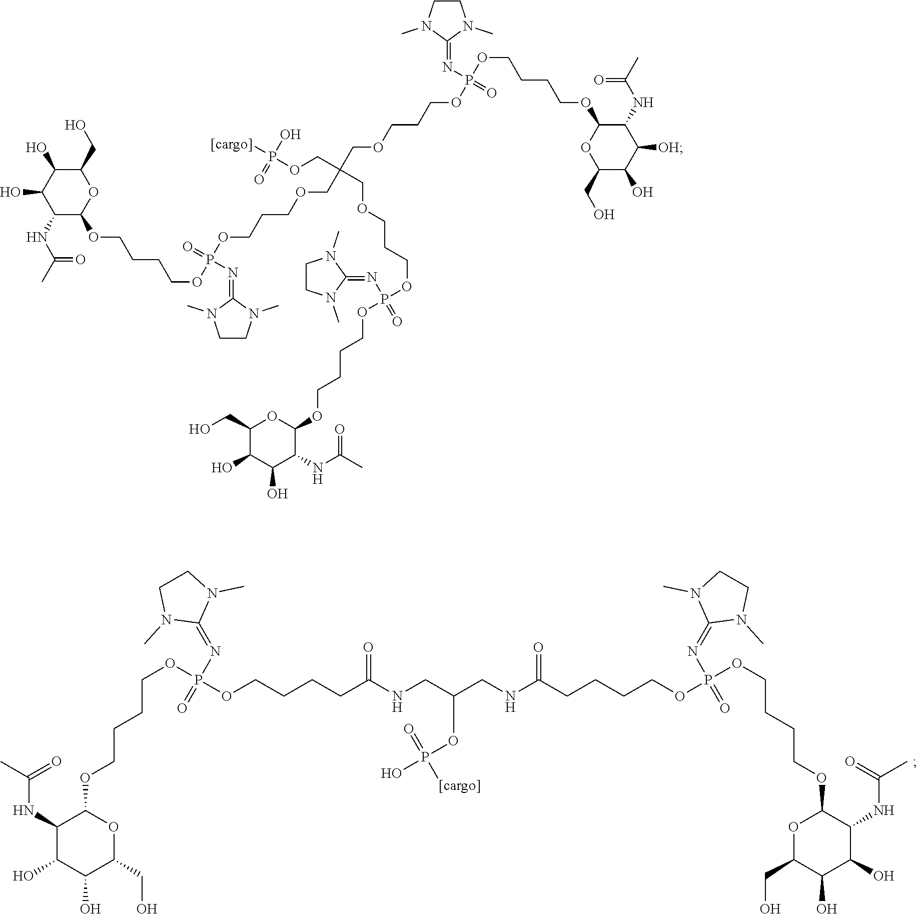 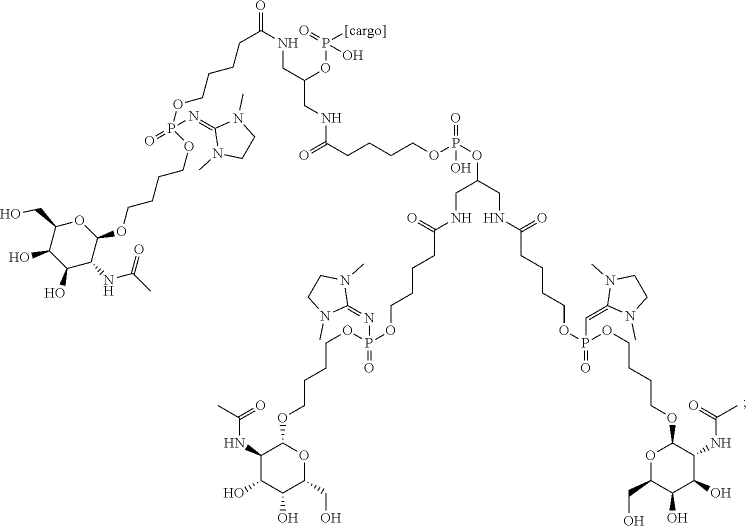 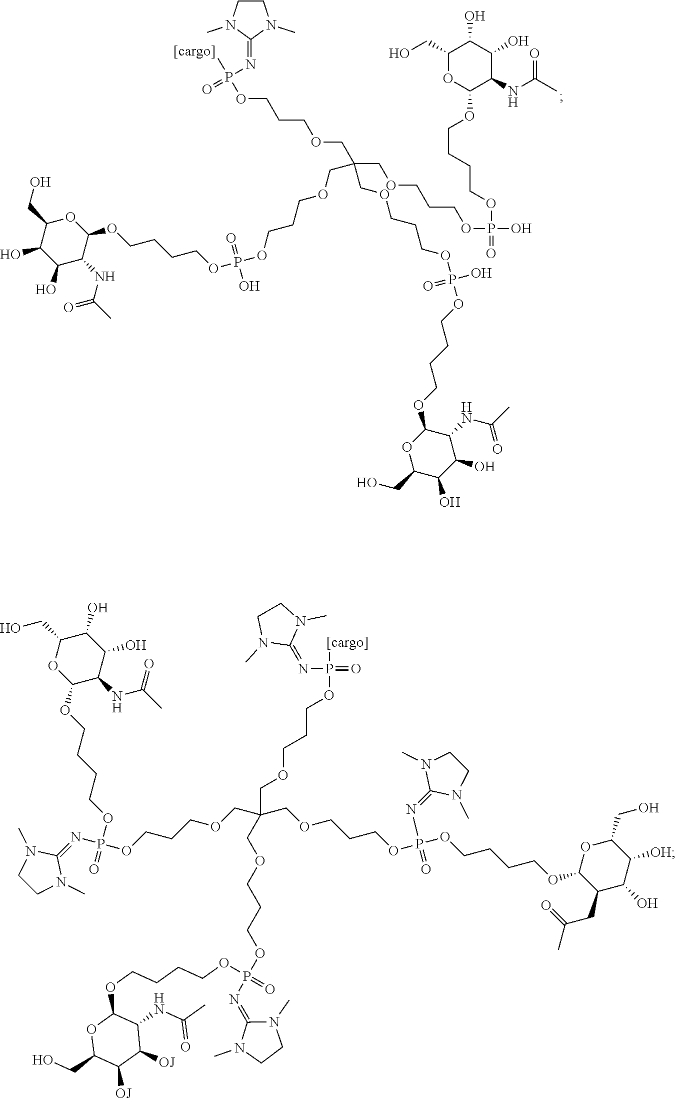 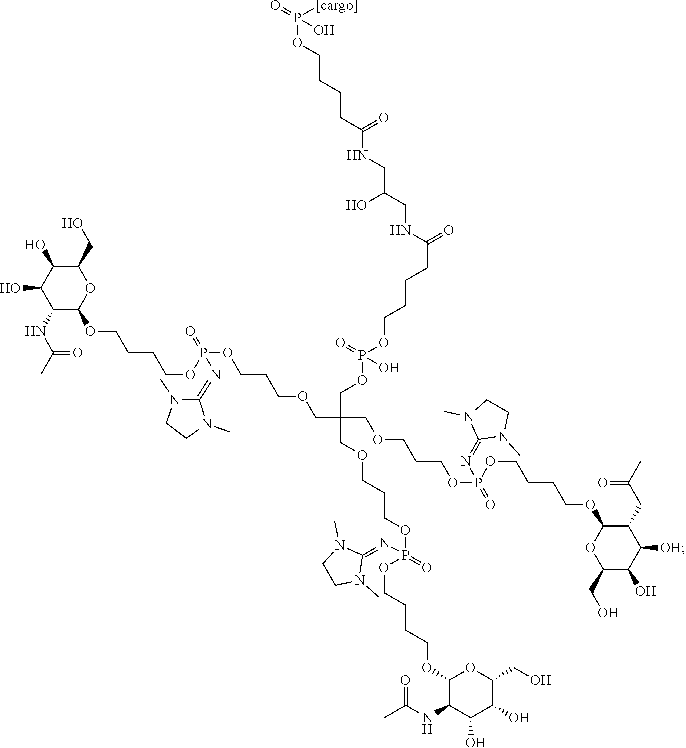 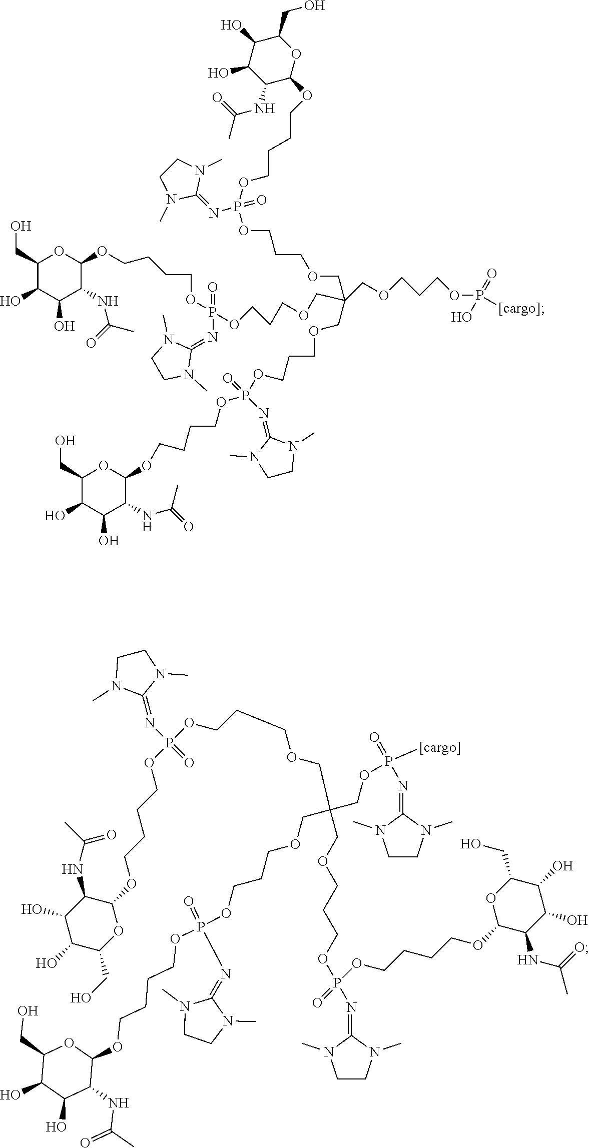
and pharmaceutically acceptable salts thereof, wherein [cargo] denotes a cargo moiety comprising a nucleic acid.

47.-48. (canceled)

49. A pharmaceutical composition comprising the nucleic acid delivery agent of claim 1, and at least one pharmaceutically acceptable excipient or carrier.

50. (canceled)

51. A method of treating a condition in a subject in need thereof, the method comprising administering an effective amount of the nucleic acid delivery agent of claim 1 to the subject, wherein the condition is selected from liver disease, genetic disease, haemophilia and bleeding disorders, liver fibrosis, non-alcoholic steatohepatitis, non-alcoholic fatty liver disease, viral hepatitis, rare diseases, metabolic disease, cardiovascular disease, obesity, thalassemia, liver injury, hemochromatosis, alcoholic liver disease, alcohol dependence, anaemia, and anaemia of chronic disease.

52. The method of claim 51, wherein the condition is selected from non-alcoholic steatohepatitis, non-alcoholic fatty liver disease, a metabolic disease, and a cardiovascular disease.

53. The method of claim 51, wherein the condition is non-alcoholic steatohepatitis.

54. The method of claim 51, wherein the condition is a metabolic disease selected from hypercholesterolemia, dyslipidaemia, and hypertriglyceridemia.
